(19) United States
(12) Patent Application Publication
(10) Pub. No.: US 2005/0222225 A1
(43) Pub. Date: Oct. 6, 2005

(54) Use of compounds for increasing spermatozoa motility

(75) Inventor: Giampiero De Luca, Conches (CH)

Correspondence Address: OBLON, SPIVAK, MCCLELLAND, MAIER & NEUSTADT, P.C., 1940 DUKE STREET, ALEXANDRIA, VA, 22314, US

(73) Assignee: APPLIED RESEARCH SYSTEMS ARS HOLDING NV, Curacao (NL)

(21) Appl. No.: 10/519,685

(22) PCT Filed: Jul. 10, 2003
(86) PCT No.: PCT/EP03/50303

§ 371 Date: May 4, 2005

(30) Foreign Application Priority Data
Jul. 10, 2002 (EP) 02100799.2
Dec. 23, 2002 (EP) 02102876.6

(51) Int. Cl. A61K 31/426; A61K 31/421; A61K 31/4164

(52) U.S. Cl. 514/369; 514/376; 514/389

(57) ABSTRACT

The invention relates to a process for the improvement of spermatozoa fertilization activity, in particular for the increase of spermatozoa motility, by using a compound of formula (I). The invention further relates to uses and methods of compounds of formula (I) in infertility and assisted reproduction techniques (ART) as well as to a medium for storage and/or transportation of spermatozoa comprising said P13K inhibitors.

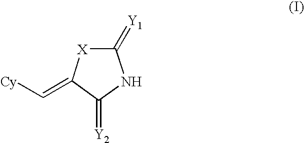

### FIELD OF THE INVENTION

The invention relates to a process for the improvement of spermatozoa fertilization activity, in particular for the increase of spermatozoa motility by using a compound of formula (I). The invention further relates to the use of a compound of formula (I) in the treatment of infertility and assisted reproduction techniques as well as methods of use thereof, and to a medium for storage and/or transportation of spermatozoa comprising the use of a compound of formula (I).

### BACKGROUND OF THE INVENTION

The infertility of a couple is defined as the inability of the woman to conceive after at least a year of regular unprotected sexual relations. Infertility may be caused by a multitude of factors, in which male factors play a fundamental role in around 40-50% of cases. Reduced male fertility is generally linked to alterations in seminal parameters such as morphology, motility and sperm count.

Various assisted reproduction techniques (ARTs) are proposed as treatment for infertility of the couple, in many cases making it possible to overcome the problem of both male and female factors. These methods, the choice of which depends on the type of diagnosis made, may involve the collection of male and female gametes (spermatozoa and oozytes). The further treatment varies according to the cause of the infertility. The gametes may be transferred directly into the Fallopian tube (GIFT=Gamete Intra Fallopian Transfer) or are brought into contact with each other in a test tube. If the latter leads to fertilization of the oozyte, the resulting zygote or embryo is transferred into the uterus (IVFET=In Vitro Fertilization and Embryo Transfer).

When infertility is due to male factor(s), parameters of the seminal liquid and in particular the count and motility of spermatozoa determine the choice of the particular assisted fertilization method used. In the most serious cases of male-factor infertility the spermatozoa count and/or their motility is very low. The fertilization activity of semen is usually assessed in a spermogram. According to WHO standards, which can be taken from the “WHO manual” (WHO laboratory manual for the examination of human semen and sperm-cervical mucus interactions, 4^{th} edtition, Cambridge University Press 1999), semen are classified into the following groups:
Normozoospermia: When all the spermatozoal parameters are normal together with normal seminal plasma, WBCs (White blood cells) and no agglutination;
Oligozoospermia: When sperm concentration is <20 million/ml;
Teratozoospermia: Fewer than 50% spermatozoa with forward progression (categories (a) and (b)) or fewer than 25% spermatozoa with category (a) movement;
Asthenozoospermia: Fewer than 50% spermatozoa with normal morphology;
Oligoasthenoteratozoospermia: Signifies disturbance of all the three variables (combination of only two prefixes may also be used);
Azoospermia: No spermatozoa in the ejaculate.

Normal values of semen parameters have been issued by WHO that are generally used as reference. The fraction of motile sperm in semen is measured either by manual counting or using a computer assisted semen analysis (CASA) system. Motility is assessed at the time of semen liquefaction and after 1 and 3 hours to detect asthenozoospermia. Manual counting classifies sperm cells into 4 categories (immotile, locally motile, non linear and linear motile) using qualitative subjective criteria of selection. Many infertility centers now use CASA systems for objective measurements of sperm motion and positive correlations have been found between motion parameters such as the amplitude of lateral head displacement, curvilinear velocity, linearity and straight-line velocity and fertilization rates in vitro but the threshold levels for these motion characteristics have not yet been established to meet a general consensus.

In case of severe male factor infertility, micro-assisted fertilization techniques can be used. Among these techniques, intracytoplasmatic sperm injection (ICSI) is the most common and has the highest percentage of success. However, the safety of the ICSI procedure for the health of the resulting conceptus or embryo is still matter of debate (Nature Medicine 5, 377-378 (1999) by Edwards R G). In addition, ICSI is far more expensive and more time consuming as compared to IVF.

Thus, the possibility to recover a higher number of spermatozoa showing a higher motility could allow several oligoasthenospermic men to enter IVF rather than ICSI programs.

Various methods have attempted at increasing the motility of the spermatozoa, like treatment of spermatozoa with pentoxyphylene, platelet activating factor or progesterone, for instance. However, the results obtained are variable and the responsiveness of the spermatozoa is not predictable.

Therefore, the finding of new methods and agents to improve sperm cell motility, leading to an improvement of the fertilization activity or fertilization rate, is highly desirable and urgently needed. These are objects of the invention to provide new methods and process to improve said sperm cell motility by using specific phosphatidylinositol-3-kinases inhibitors.

These phosphatidylinositol-3-kinases (PI3Ks) belong to a family of enzymes involved in signal transduction of tyrosine kinase receptors. Phosphatidylinositol-3-kinases, also called phosphoinositide-3-kinases (PI3Ks) generate lipids which are implicated in receptor-stimulated signalling and in the regulation of membrane traffic. Several distinct classes of PI3Ks have been identified that have been conserved throughout eukaryotic evolution. Potential signalling pathways downstream of PI3Ks have been elucidated and PI3K function is being characterized in several model organisms, as reviewed e.g. by Vanhaesebroeck et al. (Trends Biochem. Sci. 22 p. 267-72 (1997)). PI3Ks are heterodimeric enzymes present in various isoforms and composed of a catalytic subunit of 110 kDa, which is associated with a regulating subunit of 85 kDa.

In somatic cells phosphoinositide-3-kinases (PI3-kinases) are activated upon interaction with both receptor tyrosine kinases (RTK) and G-proteins resulting in the production of moieties involved in the inositol phospholipid signalling pathway. The enzyme is also present and active in human spermatozoa.

Several selective inhibitors of PI3 Ks have been described. Wortmannin is one of the most well-known specific inhibitors. Wortmannin is a fungal metabolite derived from T. wortmanin (Kyowa Hakko Kohyo Co. Ltd.) or from P. fumiculosum (Sigma). Wortmannin to and analogs thereof have already been described in patent literature (e.g. EP0635268 A1, EP0648492 A2 or EP0658343 A1). These compounds are known to be involved in the treatment of neoplasms, atherosclerosis, and bone disorders. Other phosphatidylinositol-3-kinase inhibitors are 2-(4-morpholinyl)-8-phenyl-4H-1-benzopyran-4-one (LY294002), and bioflavonoid quercetin for example described in Vlahos et al. in (J. Biol. Chem. 269, p. 5241-48 (1994)) and (J. Immunol. 154, p. 2413-22 (1995)).

The use of PI3K inhibitors in a process for the improvement of spermatozoa fertilization activity as well as for the preparation of a pharmaceutical composition in the treatment of infertility, particularly male infertility, has been disclosed by Applied Research Systems ARS Holding N.V. (WO 01/07021). In said patent, PI3K inhibitors are selected from the to group consisting of 2-(4-morpholinyl)-8-phenyl-4H-1-benzopyran-4-one (LY294002), wortmannin, quercetin and derivatives and analogues thereof.

It has now been found in accordance with the invention that phosphatidylinositol-3-kinase inhibitors of formula (I) can improve the parameters determining sperm cell fertilization activity, in particular the sperm cell motility.

### SUMMARY OF THE INVENTION

The invention therefore relates to a method of enhancing spermatozoa fertilization activity, in particular of increasing the motility of the spermatozoa, comprising the step of treating the spermatozoa by using a compound of the following formula (I) 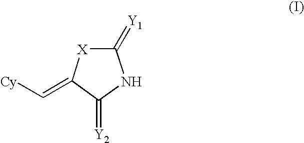
wherein X, Y¹, Y² and Cy are defined in detail in the description below.

The invention further relates to spermatozoa in which the activity of the phosphatidylinositol-3 kinase is inhibited, as well as the use of a compound according to formula (I) for improving the fertilization rate in assisted reproduction techniques (ART). A third aspect of the invention concerns the use of a compound of formula (I) for the preparation of a pharmaceutical composition for the treatment of infertility, in particular male infertility. A fourth aspect of the present invention relates to methods of ART therapy comprising treating spermatozoa with a compound of formula (I). A fifth aspect of the invention relates to a medium for storage and/or transportation of spermatozoa containing a compound of formula (I).

### DESCRIPTION OF THE INVENTION

The following paragraphs provide definitions of the various chemical moieties that make up the compounds according to the invention and are intended to apply uniformly throughout the specification and claims unless an otherwise expressly set out definition provides a broader definition.

“C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“Heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Preferred heterocycloalkyl include pyrrolidine, piperidine, piperazine, 1-methylpiperazine, morpholine, and the like.

“C₁-C₆-alkyl cycloalkyl” refers to C₁-C₆-alkyl groups having a cycloalkyl substituent, including cyclohexylmethyl, cyclopentylpropyl, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxy” refers to the group —C(O)OH.

“C₁-C₆-alkyl carboxy” refers to C₁-C₆-alkyl groups having an carboxy substituent, including 2-carboxyethyl and the like.

“Acyl” refers to the group —C(O)R where R includes “C₁-C₆-alkyl”, “aryl”, “heteroaryl”, “C₁-C₆-alkyl aryl” or “C₁-C₆-alkyl heteroaryl”.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to hetereoaryl groups having an acyl substituent, including 2-acetylpyridyl and the like.

“C₃-C₈-(hetero)cycloalkyl acyl” refers to 3 to 8 memebered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, heterocycloalkyl “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl” or “aryl” or “heteroaryl” or “C₁-C₆-alkyl aryl” or “C₁-C₆-alkyl heteroaryl”. Preferred alkoxy groups include by way of example, methoxy, ethoxy, phenoxy and the like.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes H, “C₁-C₆-alkyl” or “aryl” or “heteroaryl” or “C₁-C₆-alkyl aryl” or “C₁-C₆-alkyl heteroaryl”.

“C₁-C₆-alkyl alkoxycarbonyl” refers to C₁-C₅-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen or C₁-C₆-alkyl or aryl or heteroaryl or “C₁-C₆-alkyl aryl” or “C₁-C₆-alkyl hetero-aryl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Amino” refers to the group —NRR′ where each R, R′ is independently hydrogen or “C₁-C₆-alkyl” or “aryl” or “heteroaryl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl amino” refers to C₁-C₅-alkyl groups having an amino substituent, including 2-(1-pyrrolidinyl)ethyl and the like.

“Ammonium” refers to a positively charged group —N⁺RR′R″, where each R, R′, R″ is independently “C₁-C₆-alkyl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₅-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyl” refers to C₁-C₅-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₅-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynytheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₅-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonylamino” refers to C₁-C₅-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “carbamate”, “heteroaryl”, “sulfinyl”, “sulfonyl”, “alkoxy”, “sulfanyl”, “halogen”, “carboxy”, trihalomethyl, cyano, hydroxy, mercapto, nitro, and the like. Alternatively said substitution could also comprise situations where neighbouring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

“Pharmaceutically acceptable cationic salts or complexes” is intended to define such salts as the alkali metal salts, (e.g. sodium and potassium), alkaline earth metal salts (e.g. calcium or magnesium), aluminium salts, ammonium salts and salts with organic amines such as with methylamine, dimethylamine, trimethylamine, ethylamine, triethylamine, morpholine, N-Me-D-glucamine, N,N′-bis(phenylmethyl) 1,2-ethanediamine, ethanolamine, diethanolamine, ethylenediamine, N-methylmorpholine, piperidine, benzathine(N,N′-dibenzylethylenediamine), choline, ethylene-diamine, meglumine(N-methylglucamine), benethamine(N-benzylphenethylamine), diethylamine, piperazine, thromethamine(2-amino-2-hydroxymethyl-1,3-propanediol), procaine as well as amines of formula —NR,R′,R″ wherein R, R′, R″ is independently hydrogen, alkyl or benzyl. Especially preferred salts are sodium and potassium salts.

“Pharmaceutically acceptable salts or complexes” refers to salts or complexes of the below-identified compounds of formulae (I), (I′), (Ia), (Ib), (Ic), (Id), (II) or (III) that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. Said compounds can also be administered as pharmaceutically acceptable quaternary salts known by a person skilled in the art, which specifically include the quarternary ammonium salt of the formula —NR,R′,R″⁺Z⁻, wherein R, R′, R″ is independently hydrogen, alkyl, or benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, and Z is a counterion, including chloride, to bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” refers to any compound that upon administration to the recipient, is capable of providing directly or indirectly, the activity disclosed herein.

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

“Spermatozoa” or “sperm (cells)” are used synonymously herein and relate to male gametes. “Semen” or “seminal fluid/liquid” contain sperm cells as well as seminal plasma.

“Increase of spermatozoa fertilization activity” refers to any enhancement, improvement, or change to the better of the parameters determining the quality or activity of the sperm cell, such as e.g. percentage curvilinear velocity (VCL), average path velocity (VAP), straight-line velocity (VSL) and hyperactivated sperm fraction (HA). The quality of the spermatozoa determines the fertilization rate in assisted reproduction techniques.

“Increase of spermatozoa motility” refers to any improvement, enhancement, amelioration or change to the better of the quality or fertilization activity or motility or velocity of the cells.

“Phosphatidylinositol-3-kinase” or “PI3 K” refers to any member of the PI3K family, i.e. those related enzymes having the activity outlined in the indroduction.

“Inhibitor of phosphatidylinositol-3-kinase” refers to as PI3K and inhibits the production of D-3 phosphoinositides in the cell. The term D-3 phosphoinositides is intended to encompass derivatives of phosphatidylinositol that are phosphorylated in the D-3 position of the inositol ring and comprises, for example, phosphatidylinositol(3)monophosphate (PI(3)P), phosphatidylinositol(3,4)bisphosphate (PI(3,4)P₂) or phosphatidylinositol-(3,4,5)trisphosphate (PI(3,4,5)P₃).

“Effective amount” refers to an amount of the active ingredients that is sufficient to affect the fertilization activity, in particular the mobility of spermatozoa. The effective amount will depend on the route of administration and the condition of the patient.

“Pharmaceutically acceptable” refers to any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Besides the pharmaceutically acceptable carrier, the compositions of the invention can also comprise minor amounts of common additives, such as stabilisers, excipients, buffers and preservatives.

According to the present invention, said process to improve the spermatozoa fertilization activity, in particular for increasing spermatozoa motility, comprises the step of treating spermatozoa with a compound of formula (I).

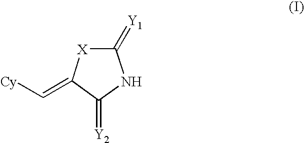

Formula (I) also comprises its geometrical isomers, its optically active forms as enantiomers, diastereomers and its racemate forms, as well as pharmaceutically acceptable salts and pharmaceutically active derivatives thereof. Preferred pharmaceutically acceptable salts of the formula (I) are acid addition salts formed with pharmaceutically acceptable acids, like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

The compounds of the present invention may be obtained as E/Z isomer mixture or as essentially pure E-isomers or Z isomers. The E/Z isomerism preferably refers to the vinyl moiety linking the phenyl with the azolidinone moiety. In a specific embodiment, the compounds of formula (I) are Z-isomers.

Such compounds of formula (I) may be used for the preparation of a pharmaceutical composition to improve the spermatozoa fertilization activity, in particular to increase spermatozoa motility and for the treatment of spermatozoa.

The substituents within formula (I) are defined as follows:

X is S, O or NH, preferably S.

Y¹ and Y² are independently S, O or —NH, preferably O.

Cy is a substituted or unsubstituted 5 to 8 membered carbocyclic or heterocyclic group which may be optionally fused with an aryl, heteroaryl, cycloalkyl or heterocycloalkyl ring.

According to a more specific embodiment of the invention, the compounds of formula (I) have a fused phenyl moiety thus giving compounds of formula (I′).

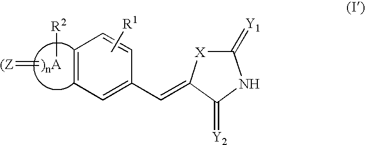

The substituents within formula (I′) are defined as follows:
A is an unsubstituted or substituted 5-8 membered heterocyclic group or an unsubstituted or substituted carbocyclic group.

Said carbocyclic group may be fused with an unsubstituted or substituted aryl, an unsubstituted or substituted heteroaryl, an unsubstituted or substituted cycloalkyl or an unsubstituted or substituted heterocycloalkyl.

Such heterocyclic or carbocyclic groups comprise aryl, heteroaryl, cycloalkyl and heterocycloalkyl, including phenyl, phenantrenyl, cyclopentyl, cyclohexyl, norbornyl, pyrrolidine, piperidine, piperazine, 1-methylpiperazine, morpholine, pyrrolyl, furanyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

Further examplary heterocyclic or carbocyclic groups A include unsubstituted or substituted dioxol, unsubstituted or substituted dioxin, unsubstituted or substituted dihydrofuran, unsubstituted or substituted (dihydro) furanyl, unsubstituted or substituted (dihydro)oxazinyl, unsubstituted or substituted oxazinoyl, unsubstituted or substituted pyridinyl, unsubstituted or substituted isooxazolyl, unsubstituted or substituted oxazolyl unsubstituted or substituted (dihydro)napthalenyl, unsubstituted or substituted pyrimidinyl, unsubstituted or substituted triazolyl, unsubstituted or substituted imidazolyl, unsubstituted or substituted pyrazinyl, unsubstituted or substituted thiazolyl, unsubstituted or substituted thiadiazolyl, unsubstituted or substituted oxadiazolyl.

X is S, O or NH, preferably S.

Y¹ and Y² are independently from each other selected from the group consisting of S, O or —NH, preferably O.

Z is S or O, preferably O.

R¹ is selected from the group comprising or consisting of H, CN, carboxy, acyl, C₁-C₆-alkoxy, halogen, hydroxy, acyloxy, an unsubstituted or substituted C₁-C₆-alkyl carboxy, an unsubstituted or substituted C₁-C₆-alkyl acyloxy, an unsubstituted or substituted C₁-C₆-alkyl alkoxy, alkoxycarbonyl, an unsubstituted or substituted C₁-C₆-alkyl alkoxycarbonyl, aminocarbonyl, an unsubstituted or substituted C₁-C₆-alkyl aminocarbonyl, acylamino, an unsubstituted or substituted C₁-C₆-alkyl acylamino, ureido, an unsubstituted or substituted C₁-C₆-alkyl ureido, amino, an unsubstituted or substituted C₁-C₆-alkyl amino, ammonium, sulfonyloxy, an unsubstituted or substituted C₁-C₆-alkyl sulfonyloxy, sulfonyl, an unsubstituted or substituted C₁-C₆-alkyl sulfonyl, sulfinyl, an unsubstituted or substituted C₁-C₆-alkyl sulfinyl, sulfanyl, an unsubstituted or substituted C₁-C₆-alkyl sulfanyl, sulfonylamino, an unsubstituted or substituted C₁-C₆-alkyl sulfonylamino or carbamate. In a specific embodiment R¹ is H.

R² is selected from the group comprising or consisting of H, halogen, acyl, amino, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl acylamino, an unsubstituted or substituted C₁-C₆-alkyl ureido, an unsubstituted or substituted C₁-C₆-alkyl carbamate, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, aryl, an unsubstituted or substituted C₃-C₈-cycloalkyl or heterocycloalkyl, an unsubstituted or substituted C₁-C₆-alkyl aryl, an unsubstituted or substituted C₂-C₆-alkenyl-aryl, an unsubstituted or substituted C₂-C₆-alkynyl aryl, carboxy, cyano, hydroxy, C₁-C₆-alkoxy, nitro, acylamino, ureido, sulfonylamino, sulfanyl, or sulfonyl.

n is an integer 0, 1 or 2, preferably n is 0 or 1. Most preferred is n=0.

According to a specific embodiment of the invention, R¹ and R² are both H.

X is S, Y¹ and Y² are both 0, R¹ and R² are as above defined and n is 0.

In a further specific embodiment according to the invention the compounds are of formula (Ia):

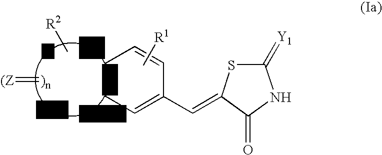

R¹, R², Y¹, Z and n in formula (Ia) are as above-defined.

G in formula (Ia) is an unsubstituted or substituted C₁-C₅ alkylene (e.g. methylene, ethylene, propylene etc.) or an unsubstituted or substituted C₁-C₅ alkenylene group (e.g. a methine (—CH═), a —CH═CH— group, a propenylene group, etc.).

W and V in formula (Ia) are each independently from each other selected from O, S, —NR³ wherein R³ is H or an unsubstituted or substituted C₁-C₆ alkyl group, m and o are each independently from each other 0 or 1; o is an integer from 1 to 4 and q is an integer from 0 to 4.

Even more preferred compounds of formula (Ia) is where G is an C₁-C₄ alkylene, thus giving compounds of formula (Ib) (i.e. p=1, 2, 3 or 4, preferably 1 or 2).

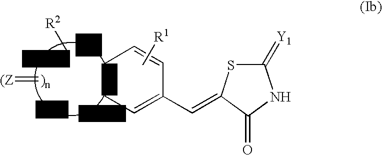

A specific sub-group of formula (Ib) are compounds having the formula (Ic), whereby W, R¹, Y¹ are as above defined; specifically R¹ may be an unsubstituted or substituted C₁-C₄-alkyl group or an unsubstituted or substituted C₁-C₅ alkenyl group, carboxy, cyano, C₁-C₄-alkoxy, nitro, acylamino, ureido.

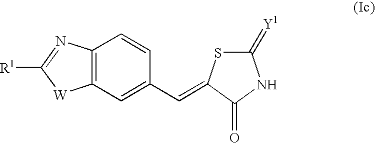

Still a further specific sub-group of formula (Ia) are compounds, wherein V, W and Y¹ are all O, thus providing compounds of formula (Id).

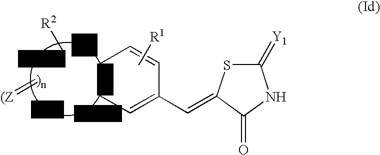

In a preferred embodiment of formulae (Ia), (Ib) or (Id), n is 0, m is 1, p is 1 or 2, o is 0, q is 1, and R¹ and R² are as above-defined.

In a further specific embodiment of formulae (Ia), (Ib) or (Id), m is 1, n is 0, p is 1 or 2, q is 0, o is 1 while R¹ and R² are as above-defined, more particularly R¹ is halogen or a hydrogen atom.

In another specific embodiment of formula (Ia), (Ib) or (Id), p is 1 or 2, q is 0, m is 0, n is 1 and R¹ and R² are as above-defined.

A further aspect of the invention consists in the use thiazolidindione-vinyl fused-benzene derivatives of formula (II-a).

More specific thiazolidinone-vinyl fused-benzene derivatives are of formula (II) 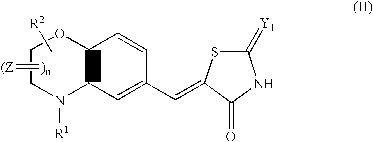
wherein Y¹, Z, R¹, R² are as above defined and n is 0 or 1.

In a specific embodiment R¹ is an unsubstituted or substituted C₁-C₆-alkyl, an unsubstituted or substituted C₁-C₆-alkyl aryl, an unsubstituted or substituted aryl, an unsubstituted or substituted C₃-C₈-cycloalkyl or -heterocycloalkyl, an unsubstituted or substituted C₁-C₆-alkyl aryl, an unsubstituted or substituted C₂-C₆-alkenyl-aryl, an unsubstituted or substituted C₂-C₆-alkynyl aryl.

In another preferred embodiment according to the present invention Y¹ is O.

More specific thiazolidinone-vinyl fused-benzene derivatives are of formula (III) 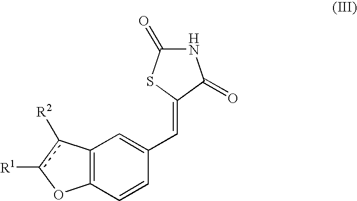
wherein R¹ and R² are as above defined.

More specific thiazolidinone-vinyl fused-benzene derivatives are of formulae (IV), (V) and (VI):

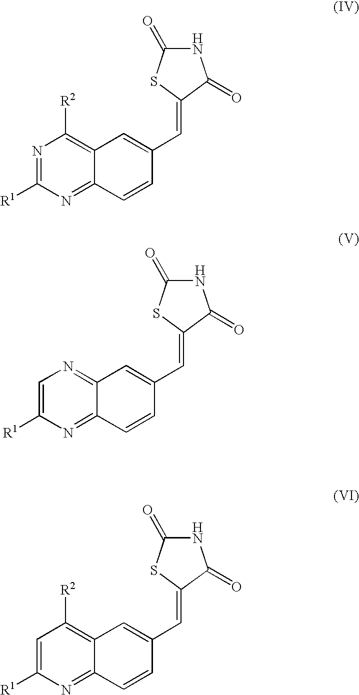

R¹ is selected from the group consisting of hydrogen, halogen, cyano, C₁-C₆-alkyl, C₁-C₆-alkoxy, acyl, alkoxy cabonyl, while R² is as above defined. In a specific embodiment R² is an amino moiety.

The compounds of the present invention are suitable for the modulation, notably the inhibition of the activity of phosphatoinositides 3-kinases (PI3K), particularly phosphatoinositides 3-kinase (PI3Kγ). It is therefore believed that the compounds of the present invention are also particularly useful for inctreasing the sperm motility.

A preferred aspect according to the invention is the one wherein the compounds of formula (I) are selected from the group consisting of:
5-(1,3-benzodioxol-5-ylmethylene)-1,3-thiazolidine-2,4-dione
5-(1,3-benzodioxol-5-ylmethylene)-2-thioxo-1,3-thiazolidin-4-one
5-(2,3-dihydro-1,4-benzodioxin-6-ylmethylene)-1,3-thiazolidine-2,4-dione
5-(2,3-dihydro-1-benzofuran-5-ylmethylene)-1,3-thiazolidine-2,4-dione
5-[(7-methoxy-1,3-benzodioxol-5-yl)methylene]-1,3-thiazolidine-2,4-dione
5-[(9,10-dioxo-9,10-dihydroanthracen-2-yl)methylene]-1,3-thiazolidine-2,4-dione
(5-[(2,2-difluoro-1,3-benzodioxol-5-yl)methylene]-1,3-thiazolidine-2,4-dione
(5Z)-5-(1,3-dihydro-2-benzofuran-5-ylmethylene)-1,3-thiazolidine-2,4-dione
5-(1-benzofuran-5-ylmethylene)-1,3-thiazolidine-2,4-dione
5-[(4-methyl-3-oxo-3,4-dihydro-2H-1,4-benzoxazin-6-yl)methylene]-1,3-thiazolidine-2,4-dione
5-(1,3-benzodioxol-5-ylmethylene)-2-imino-1,3-thiazolidin-4-one
5-Quinolin-6-ylmethylene-thiazolidine-2,4-dione
5-Quinolin-6-ylmethylene-2-thioxo-thiazolidin-4-one
2-Imino-5-quinolin-6-ylmethylene-thiazolidin-4-one
5-(3-Methyl-benzo[d]isoxazol-5-ylmethylene)-thiazolidine-2,4-dione
5-(4-Phenyl-quinazolin-6-ylmethylene)-thiazolidine-2,4-dione
5-(4-Dimethylamino-quinazolin-6-ylmethylene)-thiazolidine-2,4-dione
5-[(4-aminoquinazolin-6-yl)methylene]-1,3-thiazolidine-2,4-dione
5-[(4-piperidin-1-ylquinazolin-6-yl)methylene]-1,3-thiazolidine-2,4-dione
5-[(4-morpholin-4-ylquinazolin-6-yl)methylene]-1,3-thiazolidine-2,4-dione
5-{[4-(benzylamino)quinazolin-6-yl]methylene}-1,3-thiazolidine-2,4-dione
5-{[4-(diethylamino)quinazolin-6-yl]methylene}-1,3-thiazolidine-2,4-dione
5-({4-[(pyridin-2-ylmethyl)amino]quinazolin-6-yl}methylene)-1,3-thiazolidine-2,4-dione
5-({4-[(pyridin-3-methyl)amino]quinazolin-6-yl}methylene)-1,3-thiazolidine-2,4-dione
ethyl 1-{6-[(2,4-dioxo-1,3-thiazolidin-5-ylidene)methyl]quinazolin-4-yl}piperidine-3-carboxylate
ethyl 1-{6-[(2,4-dioxo-1,3-thiazolidin-5-ylidene)methyl]quinazolin-4-yl}piperidine-4-carboxylate
tert-butyl 1-{6-[(2,4-dioxo-1,3-thiazolidin-5-ylidene)methyl]quinazolin-4-yl}-L-prolinate
5-{[4-(4-methylpiperazin-1-yl)quinazolin-6-yl]methylene}-1,3-thiazolidine-2,4-dione
5-{[4-(4-pyrimidin-2-ylpiperazin-1-yl)quinazolin-6-yl]methylene}-1,3-thiazolidine-2,4-dione
5-({4-[4-(4-fluorophenyl)piperidin-1-yl]quinazolin-6-yl}methylene)-1,3-thiazolidine-2,4-dione
5-{[4-(4-benzylpiperidin-1-yl)quinazolin-6-yl]methylene}-1,3-thiazolidine-2,4-dione
5-({4-[4-(2-phenylethyl)piperidin-1-yl]quinazolin-6-yl}methylene)-1,3-thiazolidine-2,4-dione
5-{[4-(4-methylpiperidin-1-yl)quinazolin-6-yl]methylene}-1,3-thiazolidine-2,4-dione
5-{[4-(4-hydroxypiperidin-1-yl)quinazolin-6-yl]methylene}-1,3-thiazolidine-2,4-dione
1-[6-(2,4-Dioxo-thiazolidin-5-ylidenemethyl)-quinazolin-4-yl]-piperidine-4-carboxylic acid
1-[6-(2,4-Dioxo-thiazolidin-5-ylidenemethyl)-quinazolin-4-yl]-piperidine-3-carboxylic acid
1-[6-(2,4-Dioxo-thiazolidin-5-ylidenemethyl)-quinazolin-4-yl]-pyrrolidine-2-carboxylic acid
5-(4-Methylamino-quinazolin-6-ylmethylene)-thiazolidine-2,4-dione
5-(4-Methoxy-quinazolin-6-ylmethylene)-thiazolidine-2,4-dione
2-Imino-5-(4-methylamino-quinazolin-6-ylmethylene)-thiazolidin-4-one
2-Imino-5-(4-piperidine-quinazolin-6-ylmethylene)-thiazolidin-4-one
2-Imino-5-(4-dimethylamino-quinazolin-6-ylmethylene)-thiazolidin-4-one
5-(2-Methyl-2H-benzotriazol-5-ylmethylene)-thiazolidine-2,4-dione
5-(3-Methyl-3H-benzotriazol-5-ylmethylene)-thiazolidine-2,4-dione
5-(3-Ethyl-3H-benzoimidazol-5-ylmethylene)-thiazolidine-2,4-dione
5-{[1-(4-phenylbutyl)-1H-benzimidazol-6-yl]methylene}-1,3-thiazolidine-2,4-dione
5-[(1-prop-2-yn-1-yl-1H-benzimidazol-6-yl)methylene]-1,3-thiazolidine-2,4-dione
5-[(1-{2-[4-(trifluoromethyl)phenyl]ethyl}-1H-benzimidazol-6-yl)methylene]-1,3-thiazolidine-2,4-dione
5-({1-[2-(4-hydroxyphenyl)ethyl]-1H-benzimidazol-6-yl}methylene)-1,3-thiazolidine-2,4-dione
methyl 4-{6-[(2,4-dioxo-1,3-thiazolidin-5-ylidene)methyl]-1H-benzimidazol-1-yl}cyclohexanecarboxylate
5-({1-[2-(5-methoxy-1H-indol-3-yl)ethyl]-1H-benzimidazol-6-yl}methylene)-1,3-thiazolidine-2,4-dione
5-({1-[(1-methyl-1H-pyrazol-4-yl)methyl]-1H-benzimidazol-6-yl}methylene)-1,3-thiazolidine-2,4-dione
5-({1-[2-(3,4-dimethoxyphenyl)ethyl]-1H-benzimidazol-6-yl}methylene)-1,3-thiazolidine-2,4-dione
5-({1-[2-(4-phenoxyphenyl)ethyl]-1H-benzimidazol-6-yl}methylene)-1,3-thiazolidine-2,4-dione
5-({1-[4-(trifluoromethyl)benzyl]-1H-benzimidazol-6-yl}methylene)-1,3-thiazolidine-2,4-dione
4-{6-[(2,4-dioxo-1,3-thiazolidin-5-ylidene)methyl]-1H-benzimidazol-1-yl}cyclohexanecarboxylic acid
5-[(1-isobutyl-1H-benzimidazol-6-yl)methylene]-1,3-thiazolidine-2,4-dione
5-({1-[2-(1,3-benzodioxol-4-yl)ethyl]-1H-benzimidazol-6-yl}methylene)-1,3-thiazolidine-2,4-dione
5-({1-[2-(2-phenoxyphenyl)ethyl]-1H-benzimidazol-6-yl}methylene)-1,3-thiazolidine-2,4-dione
5-{[1-(3,3-diphenylpropyl)-1H-benzimidazol-6-yl]methylene}-1,3-thiazolidine-2,4-dione
5-{[1-(2-methoxybenzyl)-1H-benzimidazol-6-yl]methylene}-1,3-thiazolidine-2,4-dione
5-{[1-(3-furylmethyl)-1H-benzimidazol-6-yl]methylene}-1,3-thiazolidine-2,4-dione
5-[(1-propyl-1H-benzimidazol-6-yl)methylene]-1,3-thiazolidine-2,4-dione
5-Quinoxalin-6-ylmethylene-thiazolidine-2,4-dione
5-Quinoxalin-6-ylmethylene-2-thioxo-thiazolidin-4-one
2-Imino-5-quinoxalin-6-ylmethylene-thiazolidin-4-one
5-Benzothiazol-6-ylmethylene-thiazolidine-2,4-dione
5-(3-Methyl-benzofuran-5-ylmethylene)-thiazolidine-2,4-dione
5-(2-Bromo-3-methyl-benzofuran-5-ylmethylene)-thiazolidine-2,4-dione
5-(3-bromo-benzofuran-5-ylmethylene)-thiazolidine-2,4-dione
3-[5-(2,4-Dioxo-thiazolidin-5-ylidenemethyl)-benzofuran-3-yl]-acrylic acid ethyl ester
3-[5-(2,4-Dioxo-thiazolidin-5-ylidenemethyl)-benzofuran-3-yl]-acrylic acid
5-[3-(3-Oxo-3-piperidin-1-yl-propenyl)-benzofuran-5-ylmethylene]-thiazoli-dine-2,4-dione
Methyl 1-((3-{5-[(2,4-dioxo-1,3-thiazolidin-5-ylidene)methyl]-1-benzofuran-3-yl}prop-2-enoyl)prolinate
Methyl 1-((3-{5-[(2,4-dioxo-1,3-thiazolidin-5-ylidene)methyl]-1-benzofuran-3-yl}prop-2-enoyl)-D-prolinate
(5-({3-[(3-oxo-3-pyrrolidin-1-ylprop-1-en-1-yl]-1-benzofuran-5-yl}methylene)-1,3-thiazolidine-2,4-dione
5-({3-[3-morpholin-4-yl-3-oxoprop-1-en-1-yl]-1-benzofuran-5-yl}methylene)-1,3-thiazolidine-2,4-dione
Methyl 1-(3-{5-[(2,4-dioxo-1,3-thiazolidin-5-ylidene)methyl]-1-benzofuran-3-yl}prop-2-enoyl)-L-prolinate
N-cyclohexyl-3-{5-[(2,4-dioxo-1,3-thiazolidin-5-ylidene)methyl]-1-benzofuran-3-yl}-N-methylacrylamide
3-{5-[(2,4-dioxo-1,3-thiazolidin-5-ylidene)methyl]-1-benzofuran-3-yl}-N-ethyl-N-(2-hydroxyethyl)acrylamide
N-cyclobutyl-3-{5-[(2,4-dioxo-1,3-thiazolidin-5-ylidene)methyl]-1-benzofuran-3-yl}acrylamide
5-({3-[3-azetidin-1-yl-3-oxoprop-1 en-1-yl]-1-benzofuran-5-yl}methylene)-1,3-thiazolidine-2,4-dione
5-({3-[3-(1,3-dihydro-2H-isoindol-2-yl)-3-oxoprop-1-en-1-yl]-1-benzofuran-5-yl}methylene)-1,3-thiazolidine-2,4-dione
5-({3-[3-azepan-1-yl-3-oxoprop-1-en-1-yl]-1-benzofuran-5-yl}methylene)-1,3-thiazolidine-2,4-dione
3-{5-[(2,4-dioxo-1,3-thiazolidin-5-ylidene)methyl]-1-benzofuran-3-yl}-N-piperidin-1-ylacrylamide
3-{5-[(2,4-dioxo-1,3-thiazolidin-5-ylidene)methyl]-1-benzofuran-3-yl}-N-(pyridin-3-ylmethyl)acrylamide
N-cyclohexyl-3-{5-[(2,4-dioxo-1,3-thiazolidin-5-ylidene)methyl]-1-benzofuran-3-yl}acrylamide
5-({3-[3-(4-methylpiperazin-1-yl)-3-oxoprop-1-en-1-yl]-1-benzofuran-5-yl}methylene)-1,3-thiazolidine-2,4-dione
N-cycloheptyl-3-{5-[(2,4-dioxo-1,3-thiazolidin-5-ylidene)methyl]-1-benzofuran-3-yl}acrylamide
5-({3-[3-(2,5-dihydro-1H-pyrrol-1-yl)-3-oxoprop-1-en-1-yl]-1-benzofuran-5-yl}methylene)-1,3-thiazolidine-2,4-dione
N-cyclopentyl-3-{5-[(2,4-dioxo-1,3-thiazolidin-5-ylidene)methyl]-1-benzofuran-3-yl}acrylamide
3-[5-(2,4-Dioxo-thiazolidin-5-ylidenemethyl)-benzofuran-3-yl]-propionic acid ethyl ester
3-[5-(2,4-Dioxo-thiazolidin-5-ylidenemethyl)-benzofuran-3-yl]-propionic acid
5-[3-(3-Oxo-3-piperidin-1-yl-propyl)-benzofuran-5-ylmethylene]-thiazol-idine-2,4-dione
6-(2,4-Dioxo-thiazolidin-5-ylidenemethyl)-2,3-dihydro-benzo[1,4]oxazine-4-carboxylic acid tert-butyl ester
5-(3,4-Dihydro-2H-benzo[1,4]oxazin-6-ylmethylene)-thiazolidine-2,4-dione
5-(4-Benzoyl-3,4-dihydro-2H-benzo[1,4]oxazin-6-ylmethylene)-thiazolidine-2,4-dione
5-(4-Acetyl-3,4-dihydro-2H-benzo[1,4]oxazin-6-ylmethylene)-thiazolidine-2,4-dione
6-(2,4-Dioxo-thiazolidin-5-ylidenemethyl)-benzo[1,4]oxazine-4-carboxylic acid tert-butyl ester
[6-(2,4-Dioxo-thiazolidin-5-ylidenemethyl)-3-oxo-2,3-dihydro-benzo[1,4]-oxazin-4-yl]-acetic acid methyl ester
N-Benzyl-2-[6-(2,4-dioxo-thiazolidin-5-ylidenemethyl)-3-oxo-2,3-dihydro-benzo[1,4]oxazin-4-yl]-acetamide
5-(4-Butyl-3-oxo-3,4-dihydro-2H-benzo[1,4]oxazin-6-ylmethylene)-thiazoli-dine-2,4-dione
5-(4-Benzyl-3-oxo-3,4-dihydro-2H-benzo[1,4]oxazin-6-ylmethylene)-thia-zolidine-2,4-dione
5-(2-Chloro-benzofuran-5-ylmethylene)-thiazolidine-2,4-dione
5-(3-Amino-benzo[d]isoxazol-5-ylmethylene)-thiazolidine-2,4-dione
5-(3-Phenylethynyl-benzofuran-5-ylmethylene)-thiazolidine-2,4-dione
5-Benzo[1,2,5]thiadiazol-5-ylmethylene-thiazolidine-2,4-dione
5-Benzo[1,2,5]oxadiazol-5-ylmethylene-thiazolidine-2,4-dione
5-(2-Methyl-benzofuran-6-ylmethylene)-thiazolidine-2,4-dione
5-(2-Carboxymethyl-benzofuran-6-ylmethylene)-thiazolidine-2,4-dione
5-(3-Bromo-2-fluoro-2,3-dihydro-benzofuran-6-ylmethylene)-thiazolidine-2,4-dione

These agents have been shown to be particularly efficacious for the enhancement of sperm fertilization activity.

Preferably, the spermatozoa are treated with an amount of a compound of formula I in the range of about 0.01 to 1000 μM, more preferably of about 5 to 500 μM and most preferably of about 10 to 100 μM. Treating the spermatozoa with a compound of formula (I) advantageously comprises incubating the spermatozoa for a period of time in the range of about 30 minutes to 10 hours, preferably about 1 to 8 hours, most preferably about 2 to 6 hours at a temperature of about 37° C.

The invention is based on the finding that phosphatidylinositol-3-kinase inhibitors have a pronounced positive effect on parameters determining sperm cell fertilization activity, i.e. the parameters relevant to the capacity of sperm cells to fertilize an oozyte. The most important factors involved in the ability to fertilize are the number of active sperms and the motility of the spermatozoa. According to the WHO manual, motility of 50% is considered the lower limit of normality.

It has now been found in accordance with the invention that the number of motile sperms obtainable from semen samples as well as the motility of the individual spermatozoa can be significantly increased by using compounds of formula (I). This effect is detectable in normospermic individuals. However, it is even more marked in spermatozoa displaying pathogenic features, like oligoasthenospermic patients, i.e. those patients having a reduced total number of spermatozoa and a reduced spermatozoa motility. The invention renders it possible to increase the percentage of spermatozoa with progressive motility, thus significantly improving the probability of successful fertilization. Thus, the process according to the invention helps patients avoid using ICSI in favor of less invasive ART, like conventional IVF.

In a preferred embodiment, treating the spermatozoa with a compound of formula (I) is performed on the seminal liquid comprising the spermatozoa. Performing the method according to the invention directly on the seminal liquid without any further treatment has the advantage that it is simple and fast. Since the PI3K inhibitor of the invention enhances sperm cell motility, removal of the seminal plasma is not necessary.

In a further preferred embodiment, the process further comprises separating the spermatozoa by spermatozoa separation methods used in assisted reproduction techniques (ART).

Since seminal plasma contains factors that inhibit capacitation and fertilization as well as a considerable amount of non-motile spermatozoa even in a fertile individual, it is advantageous to separate motile sperm cells from fluid, non-motile and morphologically defective spermatozoa. This step is essential in traditional ART like IVF, GIFT or Intra-uterine Insemination (IUI). It leads to an enhancement of the fertilization success rate also in the process according to the invention. It is evident from the examples that the increase in spermatozoa motility by using a compound of formula (I) is even more pronounced in spermatozoa which have been separated from the seminal plasma.

In a further preferred embodiment of the invention, separating the spermatozoa is performed by a method selected from the wash and spin method, the sedimentation method, the direct swim-up method, the pellet and swim-up method, and the buoyant density gradient method. These methods are well known in the art. They are traditionally used in assisted reproduction techniques and described in detail in “A textbook of In Vitro Fertilization and Assisted Reproduction, The Bourn Hall guide to clinical and laboratory practice, editor: Peter R. Brinsden, The Parthenon Publishing Group” (1999) on pages 204 to 208. This textbook is referred to hereinafter as the “Bourn Hall guide”.

Preferably, separating the spermatozoa is performed by the direct swim-up method. This method implies self-selection of motile sperms, essentially comprising layering an aliquot of medium on top of a semen sample and allowing it to stand a room temperature for a certain period of time. The motile sperm cells will migrate into the top layer (medium), from which they can be recovered. The method may also include centrifugation step(s). The advantage of “swim-up” selected spermatozoa is that the motile cells present in the sample are isolated and concentrated and that the proportion of morphologically normal sperm is increased. It is shown in the examples that the process according to the invention leads to an increase of the amount of spermatozoa recovered from seminal fluid by the swim-up method. This is due to the increased motility of the sperms, which therefore migrate more quickly and in higher amounts into the upper phase of the sample.

The method may be varied and combined with further isolation/separation techniques, depending on the amount of motile cells in the sample. For example, the swim-up procedure may be performed through the layering of 1 ml of medium containing albumin on a 1 ml of underlying seminal liquid in a test tube. After one hour of incubation at 37° C. in the air or in 5% CO₂ the upper phase of the medium to which the spermatozoa with better motility characteristics have migrated is collected. This technique may also comprise or be combined with a centrifugation step, for example centrifugation on Percoll gradients. The separated, isolated or enriched spermatozoa are then used in assisted-reproduction techniques or may be deep-frozen before being further processed, for example.

Advantageously, the incubation of spermatozoa with a compound of formula (I) is carried out on the seminal fluid, and then swim-up selection is performed. Thereafter, the spermatozoa may be washed one or several times to eliminate the compound of formula (I), before being further processed for fertilization.

Preferably, the process according to the invention is performed on mammal spermatozoa, in particular on human spermatozoa.

The invention also relates to spermatozoa obtainable by the process described above. It is a further object of the invention to provide spermatozoa having an improved ability of fertilization. Therefore the invention further relates to spermatozoa in which the activity of the phosphatidylinositol-3 kinase is inhibited. The spermatozoa in which the a compound of formula (I) is inhibited or which were obtained in a process according to the invention exhibit an improved fertilization activity, a higher motility as compared to untreated sperm cells and thus exhibit a better performance with regard to fertilization.

As above-mentioned, sperm cell fertilization activity determines the fertilization rate in ART. The invention therefore further relates to the use of a compound of the above-mentioned formulae (I), (I′), (Ia), (Ib), (Ic), (Id), (II) or (III) for improving the fertilization rate in assisted reproduction techniques.

Any assisted reproduction method known in the art may be used according to the invention. In preferred embodiments, the assisted reproduction techniques are selected from in vitro fertilization (IVF), gamete intrafallopian transfer (GIFT), and intra-uterine insemination (IUI).

The invention further relates to the use of a compound of formula (I), (I′), (Ia), (Ib), (Ic), (Id), (II) or (III) for the preparation of a pharmaceutical composition for the treatment of infertility, in particular male infertility. While the invention is described in more detail for in vitro fertilization techniques, it will be appreciated by the person skilled in the art that the compound may be as efficient in terms of activity when administered in vivo.

In this case, the medicament is preferably presented in the form of a pharmaceutical composition comprising a compound of formula (I) together with one or more pharmaceutically acceptable carriers and/or excipients. Such pharmaceutical compositions form yet a further aspect of the present invention.

The administration of such active ingredient may be by intravenous, intramuscular or subcutaneous route. Other routes of administration, which may establish the desired blood levels of the respective ingredients, are comprised by the present invention.

The invention further relates to the use of a compound of formula (I), (I′), (Ia), (Ib), (Ic), (Id), (II) and (III) for the preparation of a pharmaceutical composition for the improvement of spermatozoa fertilization activity, in particular for the increase of spermatozoa motility.

It is a further object of the present invention to provide for an improvement concerning the method of ART therapy. The improvement consists in including into known techniques for assisted fertilization a step comprising treating spermatozoa with a compound of formula (I). The further steps used in assisted reproduction techniques are well known to the person skilled in the art and can be taken form the WHO manual (supra) or the Bourn Hall guide (supra).

In a preferred embodiment of the invention, the ART are selected from in vitro fertilization (IVF), gamete intrafallopian transfer (GIFT), or intra-uterine insemination (IUI).

It is a further object of the present invention to provide a medium for storage and/or transportation of mammal spermatozoa, particular human spermatozoa, having improved qualities. The invention therefore also relates to a medium comprising a compound of formula (I). Apart from the a compound of formula (I), the medium may contain any further component known to be useful for storage and/or transportation, depending on the kind of storage and/or transportation required. For example, the spermatozoa may be stored at room temperature or by cryo-preservation. The latter is common for the storage of the cells for a longer period of time. Specific examples of further components of the medium can be taken e.g. from WO 97/16965. Further specific media suitable for cryopreservation of semen are included in Appendix II, pp. 541 and 542 of the Bourn Hall guide (supra), for instance. They could be supplemented with a compound of formula (I) to improve the fertilization activity, in particular the motility of the sperm before fertilization takes place.

In a preferred embodiment, the medium comprises mammal spermatozoa, in particular human spermatozoa. Preferable, a compound of formula (I) present in the medium according to the invention is selected from the group consisting of (5-(2H-benzo[d]1,3-dioxolen-5-ylmethylene)-1,3-thiazolidine-2,4-dione and derivatives and analogues thereof. In a highly preferred embodiment, the compound of formula (I) is (5-(2H-benzo[d]1,3-dioxolen-5-ylmethylene)-1,3-thiazolidine-2,4-dione.

In yet a further preferred embodiment, the medium according to the invention comprises amounts of the compound of formula (I) in the range of about 0.01 to 1000 μM, preferably of about 5 to 500 μM, and most preferably of about 10 to 100 μM.

Having now described the invention, it will be more readily understood through reference to the following examples that are provided by way of illustration and are not intended to be limiting the present invention.

Compounds of formula (I), have been found—in accordance with the present invention—to be PI3K inhibitors.

The azolidinone-vinyl fused-benzene derivatives according to formula (I) are either commercially available or—as is the case for compounds of any of formulae (I′), (Ia), (Ib), (Ic), (Id), (II), (III), (IV), (V) and (VI)— may be prepared from readily available starting materials using the below set out general methods and procedures.

It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

In the process illustrated in the following schemes R¹, R², R⁴, R⁵, G, V, W, Y¹, Y², Z, m, n, o, p and q are each as above-defined in the description.

Generally, the azolidinone-vinyl fused-benzene derivatives according to the general formula (I′) could be obtained by several synthetic approaches, using both solution-phase and solid-phase chemistry protocols (Brummond et. al., J.O.C., 64, 1723-1726 (1999)), either by conventional methods or by microwave-assisted techniques.

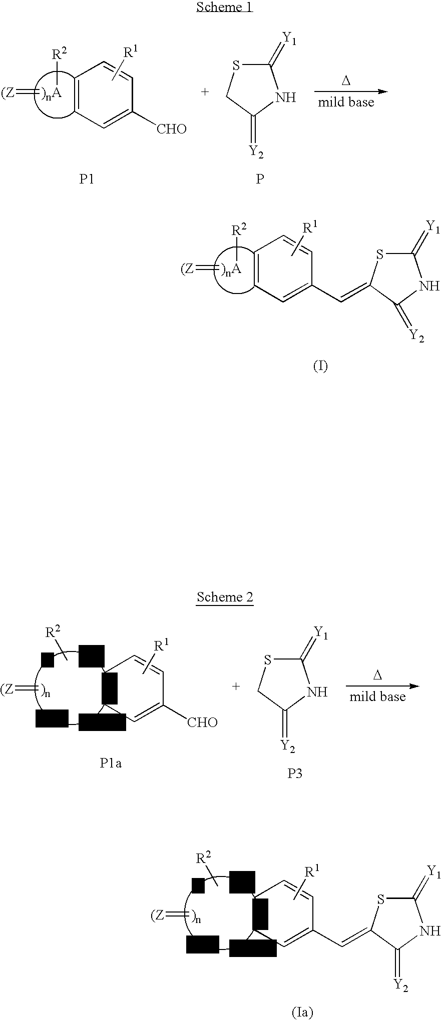

In a first step, approximately equimolar amounts of the aldehyde reactant P1 (P1a) and compound 2 (in particular thiazolidinedione or rhodanin P3) are heated in the presence of a preferably mild base to provide the corresponding olefin of formula (Ia). In the first step, P1a may be replaced by precursors P1b and P1c in order to obtain the final compounds (Ib) and (Ic) respectively as above described in the description.

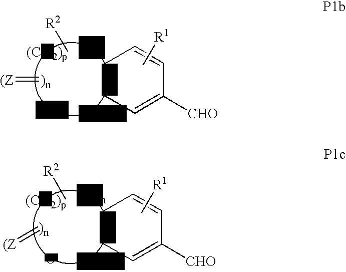

Particularly preferred process according to the invention are illustrated by the following schemes 3 and 4 in which compounds of formula (II) and (III) respectively, may be obtained using the same reaction as above-mentioned.

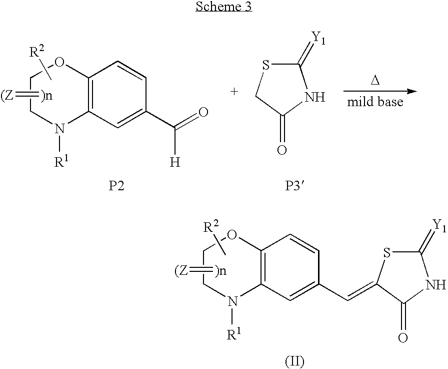

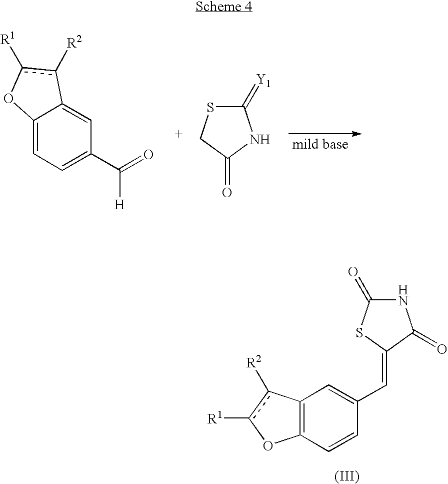

While this step may be carried out in the absence of a solvent at a temperature, which is sufficiently high to cause at least partial melting of the reaction mixture, it is preferably carried out in the presence of a inert solvent. A preferred temperature range is from about 100° C. to 250° C., and especially preferred is a temperature of from about 120° C. to 200° C. Examples of such solvents for the above reaction include solvents like dimethoxymethane, xylene, toluene, o-dichlorobenzene etc. Examples of suitable mild bases for the above reaction are alkali metal and alkaline earth salts of week acids such as the (C₁-C₁₂)-alkyl carboxylic acids and benzoic acid, alkali metal and alkaline earth carbonates and bicarbonates such as calcium carbonate, magnesium carbonate, potassium bicarbonate and secondary amines such as piperidine, morpholine as well as tertiary amines such as pyridine, triethylamine, diisopropylethylamine, N-methylmorpholine, N-ethylpiperidine, N-methylpiperidine and the like. Especially preferred mild bases are sodium acetate or piperidine for reasons of economy and efficiency.

In a typical such reaction (Tietze et. al., in “The Knoevenagel reaction”, p. 341 ff., Pergamon Press, Oxford 1991, Eds.: Trost B. M., Fleming I.) the aldehyde starting material P1a and the other starting compound (e.g. thiazolidinedione) P3 are combined in approximately equimolar amounts with 0.5 to one equivalent of piperidine in dimethoxymethane or similar solvent and heated between 120 and 200° C. at which the reaction is substantially complete in from about 15 minutes to 3 hours. The desired olefin of formula (Ia) is then isolated by filtration, in case it precipitated out of the reaction mixture upon cooling, or for example, by mixing with water and subsequent filtration, to obtain the crude product, which is purified, if desired, e.g. by crystallization or by standard chromatographic methods.

Alternatively compounds of formula (Ia) may be obtained typically by mixing equimolar amounts of thiazolidinedione P3 with aldheyde P1a and molar excess, preferably a 2-4 fold excess, of anhydrous sodium acetate and the mixture is heated at a temperature high enough to effect melting, at which temperature the reaction is mainly complete in from 5 to 60 minutes.

Preferably the above reaction is carried out in acidic media such as acetic acid in the presence of sodium acetate or beta-alanine.

Above described reactions may be carried out alternatively under microwave conditions as heating source. Typically the aldehyde starting material P1a and thiazolidinedione P3 are combined in approximately equimolar amounts with 0.5 to one equivalent of piperidine in dimethoxymethane or similar solvent and heated between 140° C. and 240° C. at which the reaction is substantially complete in from 3 to 10 minutes.

The pharmaceutically acceptable cationic salts of compounds of the present invention are readily prepared by reacting the acid forms with an appropriate base, usually one equivalent, in a co-solvent. Typical bases are sodium hydroxide, sodium methoxide, sodium ethoxide, sodium hydride, potassium hydroxide, potassium methoxide, magnesium hydroxide, calcium hydroxide, benzathine, choline, diethanolamine, ethylenediamine, meglumine, benethamine, diethylamine, piperazine and tromethamine. The salt is isolated by concentration to dryness or by addition of a non-solvent. In some cases, salts can be prepared by mixing a solution of the acid with a solution of the cation (sodium ethylhexanoate, magnesium oleate), employing a solvent in which the desired cationic salt precipitates, or can be otherwise isolated by concentration and addition of a non-solvent.

2,4-Azolidinone derivatives P3 are commercially available from various sources.

The aldehydes of formula P1a are prepared by a variety of well known methods, for example starting from the corresponding carboxylic acid alkyl ester or carboxylic acid by oxido-reduction, using standard techniques to reduce carboxylic acid alkyl ester or carboxylic acid to benzylic alcohols with lithium aluminium hydride, diisopropylaluminum etc. and ultimately re-oxidize the corresponding benzylic alcohol to the corresponding aldehyde by mild oxidation with reagents such as manganese dioxide, chromic acid, Dess-Martin reagent or Swern oxidation, or under conditions known to produce aldehydes from primary alcohols. An alternative way may be the direct reduction of the corresponding carboxylic acid alkyl ester or carboxylic acid to the corresponding aldehyde, using DIBAL at low temperature or any other techniques known in the field.

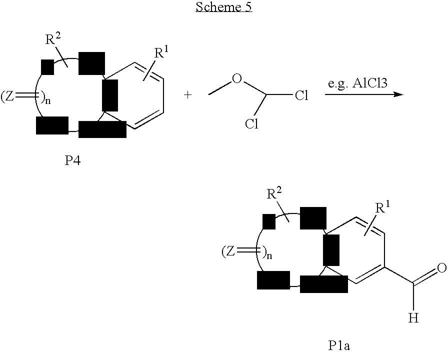

An alternative way to prepare the appropriate aldehydes is the selective reduction of a nitrile moiety to the corresponding aldehyde using known methods like e.g. DIBAL etc. Another way to access aldehydes of formula P1a is the selective reduction of the corresponding acyl chloride using e.g. Lithiumaluminiun-tri-tert-butoxyhydride (Cha J. S., Brown H. C., J.O.C 1993, 58, p. 4732-34). Another alternative way to produce the appropriate aldehydes is the reaction of the corresponding benzene derivative in a Friedl-Crafts type of reaction wherein the substrate P4 as shown in the above scheme 5 is reacted with 1,1-dichloromethylmethyl ether in the presence of a Lewis acid such as titanium tetrachloride or aluminium trichloride or any corresponding Lewis acids suitable for such type of reaction.

Acccording to a more particularly preferred process of the invention, as described in the literature (Petrov O. I., Kalcheva V. B., Antonova A. T., Collect. Czech. Chem. Commun, 62, p. 494-7 (1997)) and illustrated by Scheme 6 hereinafter, reactant P2 may be obtained starting from P5 by reacting with 1,1-dichloromethylmethyl ether as above-described.

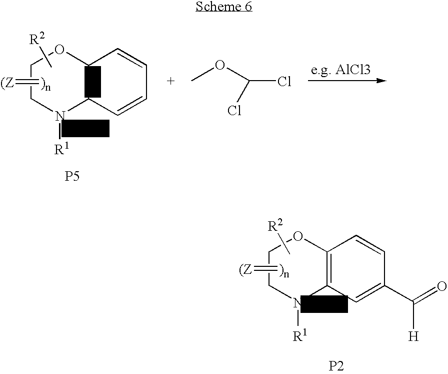

Acccording to another more particularly preferred process of the invention, as illustrated by Scheme 7 hereinafter, reactant P6 may be obtained starting from P7 by reacting with DMF and the presence of magnesium or n-butyl-lithium or any other method known to the person skilled in the art.

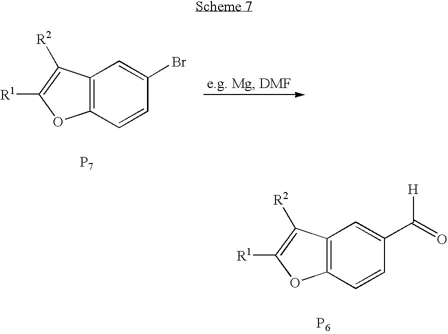

Acccording to another more particularly preferred process of the invention, as illustrated by Scheme 8 hereinafter, reactant P6 may be obtained starting from P9 by reacting n-butyllithium or LDA in the presence of an appropriate electrophile R¹—X, or any other method known to the person skilled in the art. This method may be repeated for P8 in order to obtain P6 accordingly.

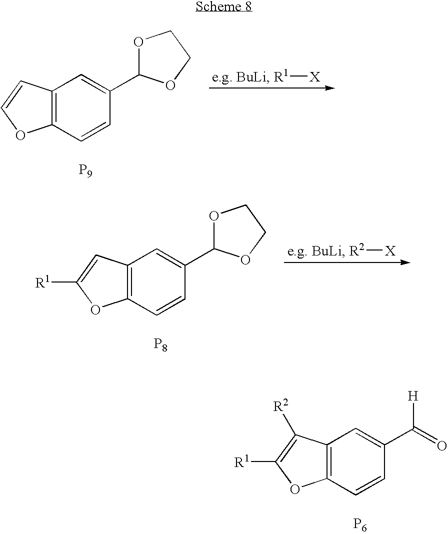

Similarily, saturated precursors P6 may be obtained in a one-pot reaction using P9 and appropriate electrophiles R¹—X and R²—X as set out in Scheme 9.

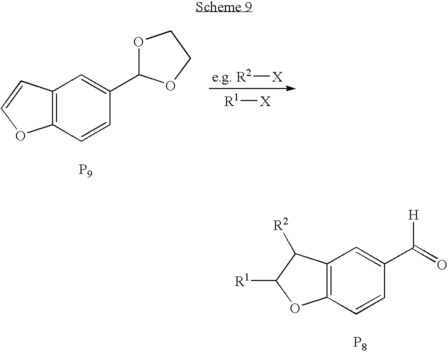

If the above set out general synthetic methods are not applicable to obtain compounds according to formula (I) and/or to necessary intermediates for the synthesis of compounds of formula (I), suitable methods of preparation known by a person skilled in the art should be used. In general, the synthesis pathways for any individual compound of formula (I) will depend on the specific substitutents of each molecule and upon the ready availability of intermediates necessary; again such factors being appreciated by those of ordinary skill in the art.

For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Compounds of this invention can be isolated in association with solvent molecules by crystallization from evaporation of an appropriate solvent. The pharmaceutically acceptable acid addition salts of the compounds of formula (I) which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of formula (I) with a suitable base. Both types of salts may be formed or interconverted using ion-exchange resin techniques.

When employed as pharmaceuticals, azolidinedione-vinyl fused-benzene derivatives of the present invention are typically administered in the form of a pharmaceutical composition. Hence, pharmaceutical compositions comprising a compound of formula (I) and a pharmaceutically acceptable carrier, diluent or excipient therefore are also within the scope of the present invention. A person skilled in the art is aware of a whole variety of such carrier, diluent or excipient compounds suitable to formulate a pharmaceutical composition.

The compounds of the invention, together with a conventionally employed adjuvant, carrier, diluent or excipient may be placed into the form of pharmaceutical compositions and to unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Pharmaceutical compositions containing azolidinedione-vinyl fused-benzene derivatives of this invention can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the thiazolidinedione-vinyl fused-benzene derivative is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dio-xide; a sweetening agent such as sucrose or saccharin; or a flavoring agent such as pepper-mint, methyl salicylate, or orange flavoring.

Injectable compositions are typically based upon injectable sterile saline or phosphate-buffered saline or other injectable carriers known in the art. As above mentioned, the thiazolidinedione-vinyl fused-benzene derivatives of formula (I) in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

In the following the present invention shall be illustrated by means of some examples which are not construed to be viewed as limiting the scope of the invention. The following abbreviations are hereinafter used in the accompanying examples: min (minute), hr (hour), g (gram), mmol (millimole), m.p. (melting point), eq (equivalents), ml (milliliter), μl (microliters), ACN (acetonitrile), Boc (butoxycarbonyl), Cbz (carboxybenzyl), CDCl₃ (deuterated chloroform), cHex (cyclohexanes), dba (dibenzylidene acetone), DCM (dichloromethane), DEAD (diethylazodicarboxylate, DIC (diisopropyl carbodiimide), DIEA (diisopropyl ethylamine), DMAP (4-dimethylaminopyridine), DME (Dimethoxyethane), DMF (dimethylformamide), DMSO (dimethylsulfoxide), DMSO-d₆ (deuterated dimethylsulfoxide), EDC (1-(3-dimethylaminopropyl)-3-ethylcarbodiimide hydrochloride), EtOAc (ethyl acetate), Et₂O (diethyl ether), Fmoc (9-fluorenylmethoxycarbonyl), HOBt (1-hydroxybenzotriazole), K₂CO₃ (potassium carbonate), MgSO₄ (magnesium sulfate), MsCl (methylsulfonyl chloride), MTBE (tert-butyl methyl ether), NaH (sodium hydride), NaHCO₃ (sodium bicarbonate), nBuLi (n-butyllithium), PCC (pyridinium chlorochromate), PetEther (petroleum ether), QCl (tetrabutylammonium chloride), rt (room temperature), TBTU (O-benzotriazolyl-N,N,N′,N′-tetramethyluronium-tetrafluoroborate), TEA (triethyl amine), TFA (trifluoroacetic acid), THF (tetrahydrofuran), TMOF (trimethylorthoformate), TMAD (N,N,N′,N′-tetramethylazodicarboxamide), TosCl (toluenesulfonyl chloride).

Example Name

1 5-(1,3-benzodioxol-5-ylmethylene)-1,3-thiazolidine-2,4-dione
2 5-(1,3-benzodioxol-5-ylmethylene)-2-thioxo-1,3-thiazolidin-4-one
3 5-(2,3-dihydro-1,4-benzodioxin-6-ylmethylene)-1,3-thiazolidine-2,4-dione
4 5-(2,3-dihydro-1-benzofuran-5-ylmethylene)-1,3-thiazolidine-2,4-dione
5 5-[(7-methoxy-1,3-benzodioxol-5-yl)methylene]-1,3-thiazolidine-2,4-dione
6 5-[(9,10-dioxo-9,10-dihydroanthracen-2-yl)methylene]-1,3-thiazolidine-2,4-dione
7 (5-[(2,2-difluoro-1,3-benzodioxol-5-yl)methylene]-1,3-thiazolidine-2,4-dione
8 (5Z)-5-(1,3-dihydro-2-benzofuran-5-ylmethylene)-1,3-thiazolidine-2,4-dione
9 5-(1-benzofuran-5-ylmethylene)-1,3-thiazolidine-2,4-dione
10 5-[(4-methyl-3-oxo-3,4-dihydro-2H-1,4-benzoxazin-6-yl)methylene]-1,3-thiazolidine-2,4-dione
11 5-(1,3-benzodioxol-5-ylmethylene)-2-imino-1,3-thiazolidin-4-one
12 5-Quinolin-6-ylmethylene-thiazolidine-2,4-dione
13 5-Quinolin-6-ylmethylene-2-thioxo-thiazolidin-4-one
14 2-Imino-5-quinolin-6-ylmethylene-thiazolidin-4-one
15 5-(3-Methyl-benzo[d]isoxazol-5-ylmethylene)-thiazolidine-2,4-dione
16 5-(4-Phenyl-quinazolin-6-ylmethylene)-thiazolidine-2,4-dione
17 5-(4-Dimethylamino-quinazolin-6-ylmethylene)-thiazolidine-2,4-dione
18 5-[(4-aminoquinazolin-6-yl)methylene]-1,3-thiazolidine-2,4-dione
19 5-[(4-piperidin-1-ylquinazolin-6-yl)methylene]-1,3-thiazolidine-2,4-dione
20 5-[(4-morpholin-4-ylquinazolin-6-yl)methylene]-1,3-thiazolidine-2,4-dione
21 5-{[4-(benzylamino)quinazolin-6-yl]methylene}-1,3-thiazolidine-2,4-dione
22 5-{[4-(diethylamino)quinazolin-6-yl]methylene}-1,3-thiazolidine-2,4-dione
23 5-({4-[(pyridin-2-ylmethyl)amino]quinazolin-6-yl}methylene)-1,3-thiazolidine-2,4-dione
24 5-({4-[(pyridin-3-ylmethyl)amino]quinazolin-6-yl}methylene)-1,3-thiazolidine-2,4-dione
25 ethyl 1-{6-[(2,4-dioxo-1,3-thiazolidin-5-ylidene)methyl]quinazolin-4-yl}piperidine-3-carboxylate
26 ethyl 1-{6-[(2,4-dioxo-1,3-thiazolidin-5-ylidene)methyl]quinazolin-4-yl}piperidine-4-carboxylate
27 tert-butyl 1-{6-[(2,4-dioxo-1,3-thiazoli din-5-ylidene)methyl]quinazolin-4-yl}-L-prolinate
28 5-{[4-(4-methylpiperazin-1-yl)quinazolin-6-yl]methylene}-1,3-thiazolidine-2,4-dione
29 5-{[4-(4-pyrimidin-2-ylpiperazin-1-yl)quinazolin-6-yl]methylene}-1,3-thiazolidine-2,4-dione
30 5-({4-[4-(4-fluorophenyl)piperidin-1-yl]quinazolin-6-yl}methylene)-1,3-thiazolidine-2,4-dione
31 5-{[4-(4-benzylpiperidin-1-yl)quinazolin-6-yl]methylene}-1,3-thiazolidine-2,4-dione
32 5-({4-[4-(2-phenylethyl)piperidin-1-yl]quinazolin-6-yl}methylene)-1,3-thiazolidine-2,4-dione
33 5-{[4-(4-methylpiperidin-1-yl)quinazolin-6-yl]methylene}-1,3-thiazolidine-2,4-dione
34 5-{[4-(4-hydroxypiperidin-1-yl)quinazolin-6-yl]methylene}-1,3-thiazolidine-2,4-dione
35 1-[6-(2,4-Dioxo-thiazolidin-5-ylidenemethyl)-quinazolin-4-yl]-piperidine-4-carboxylic acid
36 1-[6-(2,4-Dioxo-thiazolidin-5-ylidenemethyl)-quinazolin-4-yl]-piperidine-3-carboxylic acid
37 1-[6-(2,4-Dioxo-thiazolidin-5-ylidenemethyl)-quinazolin-4-yl]-pyrrolidine-2-carboxylic acid
38 5-(4-Methylamino-quinazolin-6-ylmethylene)-thiazolidine-2,4-dione
39 5-(4-Methoxy-quinazolin-6-ylmethylene)-thiazolidine-2,4-dione
40 2-Imino-5-(4-quinazolin-6-ylmethylene)-thiazolidin-4-one
41 2-Imino-5-(4-piperidine-quinazolin-6-ylmethylene)-thiazolidin-4-one
42 2-Imino-5-(4-dimethylamino-quinazolin-6-ylmethylene)-thiazolidin-4-one
43 5-(2-Methyl-2H-benzotriazol-5-ylmethylene)-thiazolidine-2,4-dione
44 5-(3-Methyl-3H-benzotriazol-5-ylmethylene)-thiazolidine-2,4-dione
45 5-(3-Ethyl-3H-benzoimidazol-5-ylmethylene)-thiazolidine-2,4-dione
46 5-{[1-(4-phenylbutyl)-1H-benzimidazol-6-yl]methylene}-1,3-thiazolidine-2,4-dione
47 5-[(1-prop-2-yn-1-yl-1H-benzimidazol-6-yl)methylene]-1,3-thiazolidine-2,4-dione
48 5-[(1-{2-[4-(trifluoromethyl)phenyl]ethyl}-1H-benzimidazol-6-yl)methylene]-1,3-thiazoli dine-2,4-dione
49 5-({1-[2-(4-hydroxyphenyl)ethyl]-1H-benzimidazol-6-yl}methylene)-1,3-thiazolidine-2,4-dione
50 methyl 4-{6-[(2,4-dioxo-1,3-thiazolidin-5-ylidene)methyl]-1H-benzimidazol-1-yl}cyclohexanecarboxylate
51 5-({1-[2-(5-methoxy-1H-indol-3-yl)ethyl]-1H-benzimidazol-6-yl}methylene)-1,3-thiazolidine-2,4-dione
52 5-({1-[(1-methyl-1H-pyrazol-4-yl)methyl]-1H-benzimidazol-6-yl}methylene)-1,3-thiazolidine-2,4-dione
53 5-({1-[2-(3,4-dimethoxyphenyl)ethyl]-1H-benzimidazol-6-yl}methylene)-1,3-thiazolidine-2,4-dione
54 5-({1-[2-(4-phenoxyphenyl)ethyl]-1H-benzimidazol-6-yl}methylene)-1,3-thiazolidine-2,4-dione
55 5-({1-[4-(trifluoromethyl)benzyl]-1H-benzimidazol-6-yl}methylene)-1,3-thiazolidine-2,4-dione
56 4-{6-[(2,4-dioxo-1,3-thiazolidin-5-ylidene)methyl]-1H-benzimidazol-1-yl}cyclohexanecarboxylic acid
57 5-[(1-isobutyl-1H-benzimidazol-6-yl)methylene]-1,3-thiazolidine-2,4-dione
58 5-({1-[2-(1,3-benzodioxol-4-yl)ethyl]-1H-benzimidazol-6-yl}methylene)-1,3-thiazolidine-2,4-dione
59 5-({1-[2-(2-phenoxyphenyl)ethyl]-1H-benzimidazol-6-yl}methylene)-1,3-thiazolidine-2,4-dione
60 5-{[1-(3,3-diphenylpropyl)-1H-benzimidazol-6-yl]methylene}-1,3-thiazolidine-2,4-dione
61 5-{[1-(2-methoxybenzyl)-1H-benzimidazol-6-yl]methylene}-1,3-thiazolidine-2,4-dione
62 5-{[1-(3-furylmethyl)-1H-benzimidazol-6-yl]methylene}-1,3-thiazolidine-2,4-dione
63 5-[(1-propyl-1H-benzimidazol-6-yl)methylene]-1,3-thiazolidine-2,4-dione
64 5-Quinoxalin-6-ylmethylene-thiazolidine-2,4-dione
65 5-Quinoxalin-6-ylmethylene-2-thioxo-thiazolidin-4-one
66 2-Imino-5-quinoxalin-6-ylmethylene-thiazolidin-4-one
67 5-Benzothiazol-6-ylmethylene-thiazolidine-2,4-dione
68 5-(3-Methyl-benzofuran-5-ylmethylene)-thiazolidine-2,4-dione
69 5-(2-Bromo-3-methyl-benzofuran-5-ylmethylene)-thiazolidine-2,4-dione
70 5-(3-bromo-benzofuran-5-ylmethylene)-thiazolidine-2,4-dione
71 3-[5-(2,4-Dioxo-thiazolidin-5-ylidenemethyl)-benzofuran-3-yl]-acrylic acid ethyl ester
72 3-[5-(2,4-Dioxo-thiazolidin-5-ylidenemethyl)-benzofuran-3-yl]-acrylic acid
73 5-[3-(3-Oxo-3-piperidin-1-yl-propenyl)-benzofuran-5-ylmethylene]-thiazoli-dine-2,4-dione
74 Methyl 1-((3-{5-[(2,4-dioxo-1,3-thiazolidin-5-ylidene)methyl]-1-benzofuran-3-yl}prop-2-enoyl)prolinate
75 Methyl 1-((3-{5-[(2,4-dioxo-1,3-thiazolidin-5-ylidene)methyl]-1-benzofuran-3-yl}prop-2-enoyl)-D-prolinate
76 (5-({3-[(3-oxo-3-pyrrolidin-1-ylprop-1-en-1-yl]-1-benzofuran-5-yl}methylene)-1,3-thiazolidine-2,4-dione
77 5-({3-[3-morpholin-4-yl-3-oxoprop-1-en-1-yl]-1-benzofuran-5-yl}methylene)-1,3′-thiazolidine-2,4-dione
78 Methyl 1-(3-{5-[(2,4-dioxo-1,3-thiazolidin-5-ylidene)methyl]-1-benzofuran-3-yl}prop-2-enoyl)-L-prolinate
79 N-cyclohexyl-3-{5-[(2,4-dioxo-1,3-thiazolidin-5-ylidene)methyl]-1-benzofuran-3-yl}-N-methylacrylamide
80 3-{5-[(2,4-dioxo-1,3-thiazolidin-5-ylidene)methyl]-1-benzofuran-3-yl}-N-ethyl-N-(2-hydroxyethyl)acrylamide
81 N-cyclobutyl-3-{5-[(2,4-dioxo-1,3-thiazolidin-5-ylidene)methyl]-1-benzofuran-3-yl}acrylamide
82 5-({3-[3-azetidin-1-yl-3-oxoprop-1-en-1-yl]-1-benzofuran-5-yl}methylene)-1,3-thiazolidine-2,4-dione
83 5-({3-[3-(1,3-dihydro-2H-isoindol-2-yl)-3-oxoprop-1-en-1-yl]-1-benzofuran-5-yl}methylene)-1,3-thiazolidine-2,4-dione
84 5-({3-[3-azepan-1-yl-3-oxoprop-1-en-1-yl]-1-benzofuran-5-yl}methylene)-1,3-thiazolidine-2,4-dione
85 3-{5-[(2,4-dioxo-1,3-thiazolidin-5-ylidene)methyl]-1-benzofuran-3-yl}-N-piperidin-1-ylacrylamide
86 3-{5-[(2,4-dioxo-1,3-thiazolidin-5-ylidene)methyl]-1-benzofuran-3-yl}-N-(pyridin-3-ylmethyl)acrylamide
87 N-cyclohexyl-3-{5-[(2,4-dioxo-1,3-thiazolidin-5-ylidene)methyl]-1-benzofuran-3-yl}acrylamide
88 5-({3-[3-(4-methylpiperazin-1-yl)-3-oxoprop-1-en-1-yl]-1-benzofuran-5-yl}methylene)-1,3-thiazolidine-2,4-dione
89 N-cycloheptyl-3-{5-[(2,4-dioxo-1,3-thiazolidin-5-ylidene)methyl]-1-benzofuran-3-yl}acrylamide
90 5-({3-[3-(2,5-dihydro-1H-pyrrol-1-yl)-3-oxoprop-1-en-1-yl]-1-benzofuran-5-yl}methylene)-1,3-thiazolidine-2,4-dione
91 N-cyclopentyl-3-{5-[(2,4-dioxo-1,3-thiazolidin-5-ylidene)methyl]-1-benzofuran-3-yl}acrylamide
92 3-[5-(2,4-Dioxo-thiazolidin-5-ylidenemethyl)-benzofuran-3-yl]-propionic acid ethyl ester
93 3-[5-(2,4-Dioxo-thiazolidin-5-ylidenemethyl)-benzofuran-3-yl]-propionic acid
94 5-[3-(3-Oxo-3-piperidin-1-yl-propyl)-benzofuran-5-ylmethylene]-thiazol-idine-2,4-dione
95 6-(2,4-Dioxo-thiazolidin-5-ylidenemethyl)-2,3-dihydro-benzo[1,4]oxazine-4-carboxylic acid tert-butyl ester
96 5-(3,4-Dihydro-2H-benzo[1,4]oxazin-6-ylmethylene)-thiazolidine-2,4-dione
97 5-(4-Benzoyl-3,4-dihydro-2H-benzo[1,4]oxazin-6-ylmethylene)-thiazolidine-2,4-dione
98 5-(4-Acetyl-3,4-dihydro-2H-benzo[1,4]oxazin-6-ylmethylene)-thiazolidine-2,4-98 dione
99 6-(2,4-Dioxo-thiazolidin-5-ylidenemethyl)-benzo[1,4]oxazine-4-carboxylic acid tert-butyl ester
100 [6-(2,4-Dioxo-thiazolidin-5-ylidenemethyl)-3-oxo-2,3-dihydro-benzo[1,4]-oxazin-4-yl]-acetic acid methyl ester
101 N-Benzyl-2-[6-(2,4-dioxo-thiazolidin-5-ylidenemethyl)-3-oxo-2,3-dihydro-benzo[1,4]oxazin-4-yl]-acetamide
102 5-(4-Butyl-3-oxo-3,4-dihydro-2H-benzo[1,4]oxazin-6-ylmethylene)-thiazolidine-2,4-dione
103 5-(4-Benzyl-3-oxo-3,4-dihydro-2H-benzo[1,4]oxazin-6-ylmethylene)-thiazolidine-2,4-dione
104 5-(2-Chloro-benzofuran-5-ylmethylene)-thiazolidine-2,4-dione
105 5-(3-Amino-benzo[d]isoxazol-5-ylmethylene)-thiazolidine-2,4-dione
106 5-(3-Phenylethynyl-benzofuran-5-ylmethylene)-thiazolidine-2,4-dione
107 5-Benzo[1,2,5]thiadiazol-5-ylmethylene-thiazolidine-2,4-ione
108 5-Benzo[1,2,5]oxadiazol-5-ylmethylene-thiazolidine-2,4-ione
109 5-(2-Methyl-benzofuran-6-ylmethylene)-thiazolidine-2,4-dione
110 5-(2-Carboxymethyl-benzofuran-6-ylmethylene)-thiazolidine-2,4-dione
111 5-(3-Bromo-2-fluoro-2,3-dihydro-benzofuran-6-ylmethylene)-thiazolidine-2,4-dione
112 5-(2-Fluoro-benzofuran-6-ylmethylene)-thiazolidine-2,4-dione

The following intermediate aldehydes are commercially available: 2,2-Difluoro-1,3-benzodioxole-5-carboxaldehyde, 1,3-Benzodioxole-5-carboxaldehyde, 1,4-Benzodioxan-6-carboxaldehyde, 9,10-Dioxo-9,10-dihydro-anthracene-2-carbaldehyde, 2,3-Dihydro-benzo[b]furan-5-carboxaldehyde, 3-Methoxy-4,5-methylenedioxybenzaldehyde.

Thiazolidinedione and Rhodanine are commercially available. Intermediate aldehydes were synthesized according to the protocols as mentioned below.

The HPLC, NMR and MS data provided in the examples described below were obtained as followed: HPLC: column Waters Symmetry C8 50×4.6 mm, Conditions: MeCN/H₂O, 5 to 100% (8 min), max plot 230-400 nm; Mass spectra: PE-SCIEX API 150 EX (APCI and ESI), LC/MS spectra: Waters ZMD (ES); ¹H-NMR: Bruker DPX-300 MHz.

The purifications were obtained as followed: Preparative HPLC Waters Prep LC 4000 System equipped with columns Prep Nova-Pak®HR C186 μm 60 Å, 40×30 mm (up to 100 mg) or 40×300 mm (up to 1 g). All the purifications were performed with a gradient of MeCN/H₂O 0.09% TFA.

### Intermediate 1: Preparation of 5-formyl-1-benzofuran

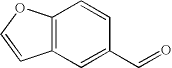

### Step I Ethyl-2-formyl-4-bromophenoxy acetate

A mixture of 5-bromosalicylaldehyde (50 g, 0.248 mol), ethylbromoacetate (42 g, 0.248 mol) and K₂CO₃ (68 g, 0.49 mol) in dry DMF (200 mL) was stirred at RT for 12 h. The reaction mixture was filtered and filtrate diluted with water. The mixture was extracted with diethylether (4×200 mL), washed with brine and concentrated to give crude ethyl-2-formyl-4-bromophenoxy acetate (64 g, 90%) as a solid.

### Step II: 4-Bromo-2-formylphenoxy acetic acid

A mixture of ethyl-2-formyl-4-bromophenoxy acetate (60 g, 0.209 mol), LiOH (7.5 g, 0.31 mol), THF (250 mL) and water (100 mL) was stirred at RT for 24 h. The reaction mixture was concentrated under reduce pressure and residue acidified with 1.5N HCl to pH=2. The solid precipitate obtained was filtered and dried to give 4-bromo-2-formylphenoxy acetic acid (50 g, 94%).

### Step III: 5-Bromo-1-benzofuran

To a mixture of 2-formyl-4-bromophenoxy acetic acid (50 g, 0.192 mol), sodium acetate (10 g, 1.21 mol) in acetic acid (250 mL) at 100° C. was added acetic anhydride (100 mL) portions during a period of 3 h. The reaction mixture was then refluxed for 20 h. The solvent was removed by distillation and residue diluted with 3N HCl (500 mL) and refluxed for 2 h. The reaction mixture was then concentrated under vacuum and product extracted with pet. ether (3×200 mL). The organic layer was washed with 10% NaHCO₃ solution and evaporated to give 5-bromo-1-benzofuran (15 g, 40%) as a pale yellow liquid.

### Step 1V: 5-Formyl-1-benzofuran (P1a in Scheme 2 for Example 9)

A mixture of 5-bromo-1-benzofuran (0.5 g), Mg (0.92 g, 0.038 mol), I₂ (1 crystal) in dry THF (2.5 mL) under N₂ atmosphere was refluxed for 30 min. To this was added a solution of 5-bromo-1-benzofuran (4.5 g) in 25 mL of dry THF) as soon as the I₂ color disappear and refluxed for another 2 h. The reaction mixture was then cooled to 40° C. and added dry DMF (3.6 g) drop-wise and slowly warmed to RT for a period of 12 h. The reaction mixture was then cooled to 0° C. and acidified with 3N HCl to pH=2 and stirred for 30 min. The reaction mixture was then diluted with water (500 mL), extracted with ethylacetate (2×200 mL), washed with brine and dried. The solvent was removed under vacuum and purified by column chromatography over silica gel (pet. ether/CH₂Cl₂) to give 5-formyl-1-benzofuran (2 g, 54%) as a liquid. LC-MS: M/Z ESI: 1.47 min, 147.34 (M+1).

### Intermediate 2: Preparation of 4-Methyl-3-oxo-3,4-dihydro-2H-benzo[1,4]oxazine-6-carbaldehyde

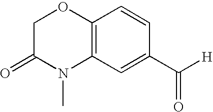

### Step I: 2-(N-methylamino)-phenol

1 g of benzoxazole was dissolved in 20 ml of THF. 0.9 g of NaBH₄ were added under nitrogen and stirring. The suspension was cooled to 0° C. and 0.86 ml of acetic acid dissolved in 5 ml THF were slowly added, keeping the reaction temperature below 5° C. The reaction was stirred at 0° C. for 30 minutes and for further 12 hours at room temperature. The reaction mixture was again cooled to 0° C. and 50 ml of sat. NH₄Cl solution were added carefully. The phases were separated and the aqueous layer extracted twice with EtOAc. The combined organic layers were washed with brine, dried over MgSO4 and filtered. Removal of the solvent afforded 0.97 g (of pure 2-(N-methylamino)-phenol.

### Step II: 4-Methyl-4H-benzo[1,4]oxazin-3-one

1 g of 2-(N-methylamino)-phenol were dissolved in chloroform, followed by the addition of 10 ml of sat. NaHCO₃ in water. To this suspension was added slowly tinder vigorous stirring a solution of 1 g of 2-chloroacetylchloride in acetone. The reaction mixture was stirred for 2 hours at room temperature. The layers were separated. The organic layer was washed with water and dried over Na₂SO₄. After evaporating the solvent, the red oil was taken up in 30 ml DMF and 1 g of K₂CO₃ were added and the slurry was heated at 70° C. for additional 2 hours. The cyclization was followed by TLC. 200 ml of EtOAc were added and the organic layer was washed 3× with 0.1N HCl and 5× with brine. The remaining organic layer was dried over MgSO4 and filtrated. EtOAc was removed under reduced pressure affording 1.45 g of pure 4-methyl-4H-benzo[1,4]oxazin-3-one.

### Step III: 4-Methyl-3-oxo-3,4-dihydro-2H-benzo[1,4]oxazine-6-carbaldehyde

1 g of AlCl₃ were suspended in 10 ml DCM, 0.5 ml of nitromethane were added to dissolve AlCl₃, and the solution was cooled to 0° C. 4-Methyl-4H-benzo[1,4]oxazin-3-one (0.5 g, 3.06 mmol) dissolved in DCM was added to the above solution and stirred for 15 minutes at 0° C. To this solution was further added 0.36 ml of bis-chloromethyl-methylether in DCM. The reaction was stirred at 0° C. for 15 minutes and at room temperature for 3 h. The crude reaction mixture was then poured onto ice, the layers were separated and the organic phase was washed with NaHCO₃ and brine. After drying over MgSO₄ and filtration the solvent was evaporated, which afforded 0.43 g of crude product. The dark oil was purified by flash chromatography using EtOAc and cyclohexane as eluents, affording 0.2 g (37%) of 4-methyl-3-oxo-3,4-dihydro-2H-benzo[1,4]oxazine-6-carbaldehyde as colourless solid.

HPLC: 2.07 min. LC-MS: M/Z ESI: 1.31 min, 192.28 (M+1).

### Intermediate 3: Preparation of 4-methyl-3,4-dihydro-2H-benzo[1,4]oxazine-7-carbaldehyde

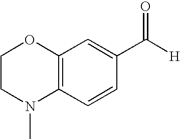

### Step I: 4-Methyl-3,4-dihydro-2H-benzo[1,4]oxazine

0.97 g of 2-(N-methylamino)-phenol were dissolved in 50 ml acetone, followed by the addition of 2 g of K₂CO₃ dissolved in water. To this suspension was added slowly a solution of 2.66 g of dibromoethane in acetone. The reaction mixture was stirred for 22 hours under reflux. Acetone was evaporated and 200 ml of EtOAc were added and the organic layer was washed 3× with 0.1N HCl and 3× with brine. The remaining organic layer was dried over MgSO4 and filtrated. EtOAc was removed under reduced pressure affording 1 g of pure 4-methyl-3,4-dihydro-2H-benzo[1,4]oxazine.

### Step II 4-Methyl-3,4-dihydro-2H-benzo[1,4]oxazine-7-carbaldehyde

4-Methyl-3,4-dihydro-2H-benzo[1,4]oxazine dissolved in 200 ul DMF under Argon. POCl₃ was added under Argon. The reaction was heated and a closed vial at 90° C. for 75 min. 1 ml of NaAc in water was added and stirred while a brown oil was formed. The oil was extracted with DCM. The organic layer was washed with brine, dried and evaporated to dryness, affording 0.18 g (76%) of 4-methyl-3,4-dihydro-2H-benzo[1,4]oxazine-7-carbaldehyde as colourless solid.

LC-MS: M/Z ESI: 1.37 min, 178.35 (M+1).

### Intermediate 4: Preparation of 1,3-Dihydroisobenzofuran-5-carbaldehyde

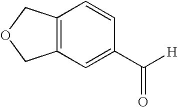

### Step I (1,3-Dihydro-isobenzofuran-5-yl)-methanol

In a round bottom flask with reflux condenser were placed 1.0 g of 3-Prop-2-ynyloxy-propyne and 2.08 g of propargylic alcohol in 10 ml ethanol, followed by the addition of 9.8 mg of tris(triphenylphosphine)rhodium chloride (Wilkinson catalyst) at room temperature. The reaction was heated up to 70° C., while the reaction colour turned yellow rapidly. After 1 day stirring at r.t., TLC analysis showed complete conversion of the starting material. The solvent was evaporated, diluted with DCM and extracted with H₂O, dried over MgSO4. The brown mixture was purified by flash chromatography using 8/2 cyclohexane/AcOEt as mobile phase affording (1,3-Dihydro-isobenzofuran-5-yl)-methanol as a colourless pure solid (0.92 g, 60%).

### Step II: 1,3-Dihydroisobenzofuran-5-carbaldehyde

(1,3-Dihydro-isobenzofuran-5-yl)-methanol (440 mg, 2.9 mmol) was dissolved in 20 ml of DCM. 1,1,1-Triacetoxy-1,1-dihydro-1,2-benziodoxol-3(1H)-one (Dess-Martin reagent) (1.3 g, 3.2 mmol) was added and the reaction was stirred at r.t. for 4 h. The reaction mixture was diluted with ether and extracted 2× with NaOH 1N, 2× with H₂O and dried over MgSO₄. The crude product was sufficiently pure and used without any further purification.

HPLC: 2.00 min. LC-MS: M/Z ESI: 1.50 min, 149.18 (M+1).

### Intermediate 5: Preparation of Quinoline-6-carbaldehyde

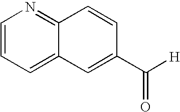

### Step I: Quinolin-6-yl-methanol

5 g of methyl quinoline-6-carboxylate was dissolved in dry THF. Under Argon was added LiAlH₄ 1M in THF (2 eq.) at −20° C. The solution was stirred at that temperature for 1 h. Isopropanol was slowly added and the crude filtered through celite and washed with DCM. Concentration gave 3.6 g (85%) of pure alcohol.

HPLC: 1.10 min. LC-MS: M/Z ESI: 0.91 min, 160.43 (M+1).

### Step II: Quinoline-6-carbaldehyde

2 g of quinolin-6-yl-methanol was dissolved in DCM. 15 g of MnO2 was added and the reaction mixture was stirred for 5 h. The crude filtered through celite and washed extensively with DCM. Concentration gave 1.85 g (93%) of pure aldehyde.

HPLC: 0.8 min. LC-MS: M/Z ESI: 1.07 min, 158.37 (M+1). ¹H NMR (DMSO-d6) δ10.19 (s, 1H), 9.06 (t, J=3 Hz, 1H), 8.6-8.66 (m, 2H), 8.15 (s, 2H), 7.68 (dd, J=3 Hz, 9 Hz, 1H).

The following intermediate was synthesized accordingly using the suitable starting materials:

### Intermediate 6: Preparation of 3-Methyl-benzo[d]isoxazole-5-carbaldehyde

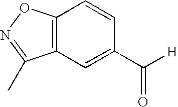

HPLC: 2.06 min. LC-MS: M/Z ESI: 1.26 min, 162.31 (M+1). ¹H NMR (DMSO-d6) δ 10.10 (s, 1H), 8.52 (s, 1H), 8.16 (d, J=12 Hz, 1H), 8.15 (s, 2H), 7.90 (d, J=9 Hz, 1H), 2.63 (s, 3H).

### Intermediate 7: Preparation of 4-Chloro-quinazoline-6-carboxylic acid methyl ester

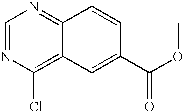

### Step I: 4-Nitro isophthalic acid

A mixture of 3-methyl-4-nitrobenzoic acid (150 g, 0.825 mol), pyridine (1.5 L) and water (1.5 L) was heated to reflux. To the hot reaction mixture was added KMnO₄ (10 mol) portion wise and reflux for 72 h. The hot reaction mixture was filtered through celite and washed with hot water. The filtrate was concentrated under vacuum, residue diluted with water (750 mL) and acidified with con. HCl at 0° C. The solid obtained was filtered, washed with water and dried under vacuum to give 4-nitro isophthalic acid (98 g, 56%).

TLC, Chloroform/Methanol, 7:3, R_{f}=0.2

### Step II: 4-Amino isophthalic acid

To a solution of 4-nitro isophthalic acid (98 g, 0.457 mol) in methanol (5 L) was added Pd/C (20%) and hydrogenated at RT for 4 h. The reaction mixture was filtered through celite and filtrate concentrated under vacuum to give 4-amino isophthalic acid (72 g, 87%) as a solid.

TLC, Chloroform/Methanol, 7:3, R_{f}=0.4

### Step III: 4-Oxo-3,4-dihydroquinazoline-6-carboxylic acid

A mixture of 4-amino isophthalic acid (17 g, 0.093 mol) and formamide (85 mL) was heated at 180° C. for 5 h. The reaction mixture was cooled to RT and added acetone. The solid precipitate thus obtained was stirred for 2 h, filtered and dried to give 4-oxo-3,4-dihydroquinazoline-6-carboxylic acid (11 g, 61%).

TLC, Chloroform/Methanol, 8:2, R_{f}=0.25

### Step 1V: 4-Oxo-3,4-dihydroquinazoline-6-methyl carboxylate

To a solution of 4-oxo-3,4-dihydroquinazoline-6-carboxylic acid (24 g, 0.126 mol) in dry methanol (800 mL) was added thionylchloride (37 g) at 5° C. and then refluxed at 80° C. for 5 h. The reaction mixture was concentrated under vacuum and crude taken in ethylacetate (250 mL). The organic layer was washed with 10% aqueous NaHCO₃, water, brine and dried. The solvent was removed under vacuum to give 4-oxo-3,4-dihydroquinazoline-6-methyl carboxylate (24 g, 92%) as a solid.

TLC, Chloroform/Methanol, 8:2, R_{f}=0.6

### Step V: Methyl-4-chloroquinazoline-6-carboxylate

A mixture of 4-oxo-3,4-dihydroquinolin-6-methyl carboxylate (12 g, 0.058 mol) and phosphorylchloride (180 mL) was heated to reflux for 7 h. Excess phosphorylchloride was distilled off and crude taken in ethylacetate (250 mL). The organic layer was washed with 10% aqueous NaHCO₃ solution, water, brine and dried. The solvent was removed under vacuum and crude purified by column chromatography over silica gel (30% ethylacetate in pet. ether) to give methyl-4-chloroquinazoline-6-carboxylate (4.5 g, 34%) as a solid.

TLC, pet. ether/EtOAc, 1:1, R_{f}=0.65

LC-MS: M/Z ESI: 1.50 min, 223.19 (M+1). ¹HNMR (DMSO-d6) δ 8.66 (d, J=1.9 Hz, 1H), 8.39 (s, 1H), 8.30 (dd, J=0.6 Hz, 8.5 Hz, 1H), 7.79 (d, J=8.5 Hz, 1H), 3.90 (s, 3H).

### Intermediate 8: Preparation of 4-Methoxy-quinazoline-6-carboxylic acid methyl ester

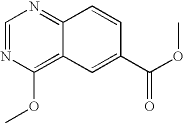

200 mg of methyl-4-chloroquinoline-6-carboxylate were strirred in 5 ml MeOH in the presence of 1 eq. of DIEA at 60° C. for 24 h. MeOH was evaporated and the crude residue was taken up in EtOAc and washed with NH₄Cl affording a white solid sufficiently pure for the next step.

HPLC: 2.3 min. LC-MS: M/Z ESI: 1.19 min, 219.17 (M+1).

The following intermediate was synthesized according to the synthesis of intermediate 8:

### Intermediate 9: Preparation of 4-Methylamino-quinazoline-6-carboxylic acid methyl ester

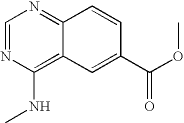

HPLC: 1.12 min. LC-MS: M/Z ESI: 1.06 min, 218.31 (M+1).

### Intermediate 10: Preparation of 4-Methoxy-quinazoline-6-carbaldehyde

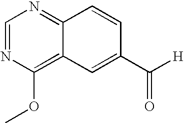

This intermediate was prepared according to the synthesis of intermediate 5 starting from 4-Methoxy-quinazoline-6-carboxylic acid methyl ester.

HPLC: 1.41 min. LC-MS: M/Z ESI: 1.24 min, 189.31 (M+1).

### Intermediate 11: Preparation of 4-Methylamino-quinazoline-6-carbaldehyde

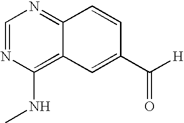

This intermediate was prepared according to the synthesis of intermediate 5 starting from 4-Methylamino-quinazoline-6-carboxylic acid methyl ester.

HPLC: 1.3 min. LC-MS: M/Z ESI: 0.90 min, 188.34 (M+1).

### Intermediate 12: Preparation of 4-Chloro-quinazoline-6-carbaldehyde

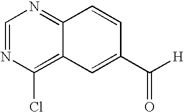

### Step I: 4-Chloroquinazoline-6-yl methanol

To a solution of methyl-4-chloroquinazoline-6-carboxylate (3.5 g, 0.015 mol) in dry THF (35 mL) at −25° C. was added DIBAL-H (4.4 g, 0.031 mol) and stirred at −25° C. to RT for 2 h. The reaction mixture was cooled to −10° C. and quenched with 10% aqueous NaHCO₃ (9 mL). The reaction mixture was extracted with ethylacetate (100 mL), washed with water, brine and dried. The solvent was removed under vacuum to give 4-chloroquinoline-6-yl methanol (2 g, 66%).

TLC, Chloroform/Methanol, 8:2, R_{f}=0.35

### Step 11: 4-Chloroquinazoline-6-carboxaldehyde

To a solution of 4-chloroquinazoline-6-yl-methanol (3.5 g, 0.018 mol) in dry CH₂Cl₂ (100 mL) was added Dess-Martin periodinane (8.4 g, 0.019 mol) and stirred at RT for 30 min. The reaction mixture was washed with 10% aqueous NaHCO₃ (75 mL), water, brine and dried. The solvent was removed under vacuum to give 4-chloroquinazoline-6-carboxaldehyde (3 g, 88%) as pale yellow solid.

TLC, Chloroform/Methanol, 9:1, R_{f}=0.6

### Intermediate 13: Preparation of 4-Phenyl-quinazoline-6-carbaldehyde

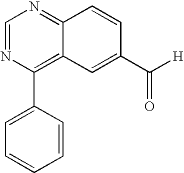

4-Chloro-quinazoline-6-carbaldehyde (50 mg, 0.26 mmol), Pd(PPh₃)₄ (13 mg, 0.01 mmol), phenylboronic acid (63 mg, 0.52 mmol) and sodium carbonate (sat. sol: 50 ul) were heated Lip in toluene at 100° C. for 12 h. After evaporation of the solvents, the residue was taken up in ethyl acetate and washed with brine twice. Organic phases were then concentrated and raw materiel was purified on silica gel using DCM/EtOH 95:5 as eluents to give 50 mgs (82%) of the desired cpd with a 85% purity.

HPLC: 2.68 min. LC-MS: M/Z ESI: 1.25 min, 235.30 (M+1).

### Intermediate 14: Preparation of 4-Dimethylamino-quinazoline-6-carbaldehyde

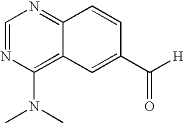

4-Chloro-quinazoline-6-carbaldehyde (200 mg, 1 mmol) was dissolved in 10 ml dioxane. To this solution was added a solution of dimethylamine in water (5 eq.). The mixture was stirred during 2 h at r.t. Evaporation of the solvents and remaining amine under high vacuum afforded pure 4-Dimethylamino-quinazoline-6-carbaldehyde as a yellow solid, which was used for the next step without further purification (190 mg=91%).

HPLC: 0.91 min. LC-MS: M/Z ESI: 1.23 min, 202.33 (M+1). ¹H NMR (CDCl3): δ10.19 (s, 1H), 8.70 (s, 1H), 8.50 (d, J=3 Hz, 1H), 8.15 (dd, J==3 Hz, 9 Hz, 1H), 7.88 (d, J=9 Hz, 1H).

The following intermediates were synthesized in a similar way using the suitable amines as nucleophiles.

| | | M/Z |
|---|---|---|
| N^{o}. | Intermediate | ESI: (M + 1). |
| 15 | 4-Piperidin-1-yl-quinazoline-6-carbaldehyde | 242.27 |
| 16 | 4-Amino-quinazoline-6-carbaldehyde | 174.18 |
| 17 | 4-Benzylamino-quinazoline-6-carbaldehyde | 264.30 |
| 18 | 4-[(Pyridin-2-ylmethyl)-amino]-quinazoline-6-carbaldehyde | 265.33 |
| 19 | 4-[(Pyridin-3-ylmethyl)-amino]-quinazoline-6-carbaldehyde | 265.33 |
| 20 | 4-(4-Methyl-piperazin-1-yl)-quinazoline-6-carbaldehyde | 257.31 |
| 21 | 4-Diethylamino-quinazoline-6-carbaldehyde | 230.28 |
| 22 | 4-Morpholin-4-yl-quinazoline-6-carbaldehyde | 244.26 |
| 23 | 1-(6-Formyl-quinazolin-4-yl)-piperidine-3-carboxylic acid ethyl | 314.36 |
| | ester | |
| 24 | 1-(6-Formyl-quinazolin-4-yl)-pyrrolidine-2-carboxylic acid tert- | 328.39 |
| | butylester | |
| 25 | 1-(6-Formyl-quinazolin-4-yl)-piperidine-4-carboxylic acid ethyl | 314.36 |
| | ester | |
| 26 | 4-(4-Hydroxy-piperidin-1-yl)-quinazoline-6-carbaldehyde | 258.30 |
| 27 | 4-(4-Methyl-piperidin-1-yl)-quinazoline-6-carbaldehyde | 256.32 |
| 28 | 4-(4-Phenethyl-piperidin-1-yl)-quinazoline-6-carbaldehyde | 346.42 |
| 29 | 4-(4-Benzyl-piperidin-1-yl)-quinazoline-6-carbaldehyde | 332.40 |
| 30 | 4-[4-(4-Fluoro-phenyl)-piperidin-1-yl]-quinazoline-6-carbaldehyde | 336.38 |
| 31 | 4-(4-Pyrimidin-2-yl-piperazin-1-yl)-quinazoline-6-carbaldehyde | 321.36 |

### Intermediates 32: Preparation of Methyl-benzotriazole-5-carboxylic acid methyl ester

1 g of Benzotriazole-5-carboxylic acid methyl ester (5.64 mmol) was dissolved in 20 ml DMF at 0° C. To this solution was added 1 eq. of NaH (60%) at 0° C. The mixture was stirred for 30 min at 0° C., 801 mg (1 eq.) of Methyliodide were slowly added, and the resulting reaction mixture was stirred for 2 h at rt. EtOAc was added and the organic layer was washed extensively with brine and water, dried over MgSO4 and filtered to afford 1 g of crude Methyl-benzotriazole-5-carboxylic acid methyl ester as three different regioisomers. The separation was performed on silica gel using EtOAc/CH 3:7 as eluents.

### Intermediate 32a: 2-Methyl-2H-benzotriazole-5-carboxylic acid methyl ester

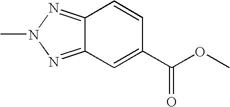

2-Methyl-2H-benzotriazole-5-carboxylic acid methyl ester eluted as first fraction (250 mg, 22%). HPLC: 2.32 min. ¹H NMR (DMSO-d6) δ8.56 (s, 1H), 8.02 (d, J=9 Hz, 1H), 7.93 (d, J=9 Hz, 1H), 4.55 (s, 3H), 3.90 (s, 1H).

### Intermediate 32b: 3-Methyl-3H-benzotriazole-5-carboxylic acid methyl ester

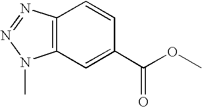

3-Methyl-3H-benzotriazole-5-carboxylic acid methyl ester eluted as 2^{nd} fraction (130 mg, 12%). HPLC: 2.03 min. ¹H NMR (DMSO-d6) δ8.56 (s, 1H), 8.13 (d, J=6 Hz, 1H), 7.93 (d, J=9 Hz, 1H), 4.39 (s, 3H), 3.92 (s, 3H).

### Intermediate 32c: 1-Methyl-1H-benzotriazole-5-carboxylic acid methyl ester

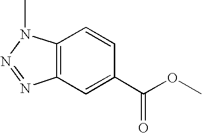

1-Methyl-1H-benzotriazole-5-carboxylic acid methyl ester eluted as 3^{rd} fraction (135 mg, 12%). HPLC: 2.03 min. ¹H NMR (DMSO-d6) δ8.62 (s, 1H), 8.11 (d, J=9 Hz, 1H), 7.97(d, 9 Hz, 1H), 4.35 (s, 3H), 3.90 (s, 3H).

### Intermediate 33: 2-Methyl-2H-benzotriazole-5-carbaldehyde

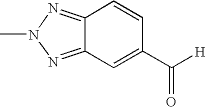

This intermediate has been synthesized according to the synthesis of intermediate 5 using 2-Methyl-2H-benzotriazole-5-carboxylic acid methyl (intermediate 32a) ester as starting point.

HPLC: 1.88 min. ¹H NMR (DMSO-d6) δ 10.12 (s, 1H), 8.65 (s, 1H), 8.06 (d, J=9 Hz, 1H), 7.85 (d, J=9 Hz, 1H), 4.57 (s, 3H).

### Intermediate 34: 3-Methyl-3H-benzotriazole-5-carbaldehyde

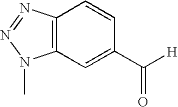

This intermediate has been synthesized according to the synthesis of intermediate 5 using 3-Methyl-3H-benzotriazole-5-carboxylic acid methyl ester (intermediate 32b) as starting point.

HPLC: 1.49 min. ¹H NMR (DMSO-d6) δ 10.18 (s, 1H), 8.54 (s, 1H), 8.20 (d, f-9 Hz, 1H), 7.88(d, J=9 Hz, 1H), 4.41 (s, 3H).

### Intermediate 35: 1-Methyl-1H-benzotriazole-5-carbaldehyde

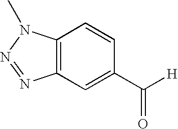

This intermediate has been synthesized according to the synthesis of intermediate 5 using 1-Methyl-1H-benzotriazole-5-carboxylic acid methyl ester as starting point (intermediate 32c).

HPLC: 1.49 min. LC-MS: M/Z ESI: 1.07 min, 162.32 (M+1). ¹H NMR (DMSO-d6) δ 10.13 (s, 1H), 8.70 (s, 1H), 8.05 (s, 2H), 4.36 (s, 3H).

### Intermediate 36: 5-(4-Amino-3-ethylamino-benzylidene)-thiazolidine-2,4-dione

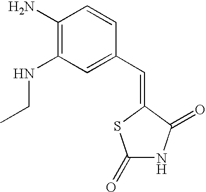

### Step I: 3-Fluoro 4-nitro benzyl alcohol (Bioorg. Med. Chem. 7, 1999, 2647)

To an ice-cooled suspension of NaBH₄ (204 mg, 5.4 mmol, 2 eq.) in THF (10 mL) was added dropwise 3-fluoro 4-nitro benzoic acid (500 mg, 2.7 mmol, 1 eq.) in THF (10 mL) over 30 minutes. BF₃-Et₂O (7.3 mmol, 2.7 eq.) was then added dropwise over 30 minutes. The solution was stirred at room temperature over night. 1N HCl was added dropwise to quench NaBH₄ excess. The solvent was removed in vacuo, the residue dissolved in DCM, washed with water, brine. The organic layer was then dried over MgSO₄ and the solvent removed in vacuo to give 425 mg of 3-fluoro 4-nitro benzyl alcohol (92% yield). The compound was used in the following step with no further purification.

¹H NMR: δ=(400 MHz, CDCl₃): 7.97 (m, 1H), 7.28 (m, 1H), 7.18 (m, 1H), 4.75 (m, 2H).

### Step II: 3-Fluoro 4-nitro benzyl aldehyde

3-fluoro 4-nitro benzyl alcohol (116 mg, 0.68 mmol, 1 eq.) was dissolved in DCM (10 ml) and treated with MnO₂ (580 mg, 6.73 mmol, 10 eq.) and the suspension stirred at room temperature over night. MnO₂ was filtered off the suspension using celite and the solvent evaporated to give the corresponding aldehyde as a white solid (66% yield).

¹H NMR: δ=(400 MHz, CDCl₃): 9.98 (s, 1H, CHO), 8.08 (m, 1H, ArH), 7.78 (m, 2H, ArH).

### Step III: 5-(3-Fluoro-4-nitro-benzylidene)-thiazolidine-2,4-dione (J. Med. Chem. 37, 2, 1994, 322)

A mixture of 3-fluoro 4-nitro benzyl aldehyde (280 mg, 1.65 mmol, 1 eq.), thiazolidinedione (193 mg, 1.65 mmol, 1 eq.) and β-alanine (95 mg, 1.1 mmol, 0.65 eq.) in acetic acid (5 mL) was stirred over night at 100° C. The cooled reaction mixture was added to water and stirred for 1 hour. The precipitated product was filtered and washed with water and dried to yield the final product as a yellow/orange solid (77% yield).

¹H NMR: δ=(400 MHz, (CD₃)₂CO): 8.0 (m, 1H, ArH), 7.68 (m, 2H, ArH), 7.53 (s, 1H, CH═C).

### Step 1V: 5-(3-Ethylamino-4-nitro-benzylidene)-thiazolidine-2,4-dione

5-(3-Fluoro-4-nitro-benzylidene)-thiazolidine-2,4-dione (200 mg, 0.75 mmol, 1 eq.), was dissolved in DME (6 mL) and TEA (208 μL, 1.5 mmol, 2 eq.) and a solution of ethylamine (2 eq.) was added. The reaction mixture was shaken at 60° C. over night. The solvent was removed in vacuo and residue dissolved in ethyl acetate and washed with 10% ammonium chloride aqueous solution. The organic layer was dried on Na₂SO₄ and the solvent evaporated to give the corresponding aniline derivative as either red oil, which was used for the next step without further purification.

### Step V: 5-(3-Ethylamino-4-amino-benzylidene)-thiazolidine-2,4-dione

To a stirred solution of 5-(3-Ethylamino-4-nitro-benzylidene)-thiazolidine-2,4-dione in THF, a solution of sodium hydrosulfite (3 eq.) in water was slowly added followed by an aqueous solution of K₂CO₃. The reaction mixture was refluxed over night. THF was removed in vacuo and residue extracted with ethyl acetate The organic layer was dried on Na₂SO₄ and the solvent evaporated to give the corresponding aniline derivative, which was used without any further purification.

The following intermediates were synthesized in a similar way using the suitable amines as nucleophiles as described in step 1V of intermediate 36. The so-obtained 3-alkylamino-4-nitro-benzylidene)-thiazolidine-2,4-diones were reduced as described in step V of intermediate 36 affording 3-alkylamino-4-amino-benzylidene)-thiazolidine-2,4-diones.

| | | M/Z |
|---|---|---|
| N^{o}. | Intermediate | ESI: (M + 1) |
| 37 | 5-[4-Amino-3-(4-phenyl-butylamino)-benzylidene]-thiazolidine-2,4-dione | 368.2 |
| 38 | 5-{4-Amino-3-[2-(4-trifluoromethyl-phenyl)-ethylamino]-benzylidene}- | 408.12 |
| | thiazolidine-2,4-dione | |
| 39 | 5-{4-Amino-3-[2-(4-hydroxy-phenyl)-ethylamino]-benzylidene}- | |
| | thiazolidine-2,4-dione | |
| 40 | 4-[2-Amino-5-(2,4-dioxo-thiazolidin-5-ylidenemethyl)-phenylamino]- | 376.35 |
| | cyclohexanecarboxylic acid methyl ester | |
| 41 | 5-{4-Amino-3-[2-(1H-indol-3-yl)-ethylamino]-benzylidene}-thiazolidine- | 409.21 |
| | 2,4-dione | |
| 42 | 5-{4-Amino-3-[(1-methyl-1H-pyrazol-4-ylmethyl)-amino]-benzylidene}- | 331.1 |
| | thiazolidine-2,4-dione | |
| 43 | 5-{4-Amino-3-[2-(3,4-dimethoxy-phenyl)-ethylamino]-benzylidene}- | 400.21 |
| | thiazolidine-2,4-dione | |
| 44 | 5-[4-Amino-3-(4-trifluoromethyl-benzylamino)-benzylidene]- | 394.15 |
| | thiazolidine-2,4-dione | |
| 45 | 4-[2-Amino-5-(2,4-dioxo-thiazolidin-5-ylidenemethyl)-phenylamino]- | 362.17 |
| | cyclohexanecarboxylic acid | |
| 46 | 5-(4-Amino-3-isobutylamino-benzylidene)-thiazolidine-2,4-dione | 292.22 |
| 47 | 5-[4-Amino-3-(2-benzo[1,3]dioxol-4-yl-ethylamino)-benzylidene]- | 384.26 |
| | thiazolidine-2,4-dione | |
| 48 | 5-{4-Amino-3-[2-(2-phenoxy-phenyl)-ethylamino]-benzylidene}- | 432.28 |
| | thiazolidine-2,4-dione | |
| 49 | 5-[4-Amino-3-(3,3-diphenyl-propylamino)-benzylidene]-thiazolidine-2,4- | 430.27 |
| | dione | |
| 50 | 5-(4-Amino-3-prop-2-ynylamino-benzylidene)-thiazolidine-2,4-dione | 274.21 |
| 51 | 5-[4-Amino-3-(2-methoxy-benzylamino)-benzylidene]-thiazolidine-2,4- | 356.23 |
| | dione | |
| 52 | 5-{4-Amino-3-[(furan-3-ylmethyl)-amino]-benzylidene}-thiazolidine-2,4- | 316.21 |
| | dione | |
| 53 | 5-(4-Amino-3-propylamino-benzylidene)-thiazolidine-2,4-dione | 278.16 |
| 54 | 5-{4-Amino-3-[2-(4-phenoxy-phenyl)-ethylamino]-benzylidene}- | 432.23 |
| | thiazolidine-2,4-dione | |

### Intermediate 55: Quinoxaline-6-carbaldehyde

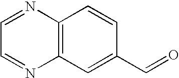

### Step I: Quinoxaline-6-carbonyl chloride

In a 1 l 3 neck flask was placed Quinoxaline-6-carboxylic acid (20.2 g) in 500 ml of THF. To this solution was slowly added thionylchloride (42 ml, 5 eq.). The reaction mechanically stirred was warmed up to reflux and followed by HPLC quenching the sample with NH₄OH. After 3 h at reflux no more starting material was present, the solvent was removed under reduced pressure and SOCl₂ was chased with toluene 3 times. The solid was suspended in 100 ml EtOAc and filtered to obtain 23.47 g of a beige solid.

HPLC: 1.114 min. ¹H NMR (DMSO-d6) δ 9.01-7.40 (m, 5H).

### Step I: Quinoxaline-6-carbaldehyde

In a 1 l 3-neck flask under argon was placed Quinoxaline-6-carbonyl chloride in 600 ml of DME. To this solution was added lithium tri-tert-butoxyaluminohydride (1 Eq.) at −78° C. over 1.5 h. The reaction was kept at that temperature for 5 h. Then ice was added, and the reaction was diluted with AcOEt and filtrated over celite. The two layers were separated and the organic phase was washed with NaHCO₃ sat. Quinoxaline-6-carbaldehyde was obtained upon evaporating the solvent in 73% yield as yellowish solid.

HPLC: 1.49 min. LC-MS: M/Z ESI: 0.81 min, 159.37(M+1). ¹H NMR (CDCl3) δ 10.28 (s, 1H), 8.97 (s, 2H), 8.61 (s, 1H), 8.27 (q, 6 Hz, 9 Hz, 2H).

### Intermediate 56: Benzothiazole-6-carbaldehyde

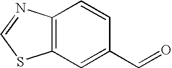

This intermediate was synthesized as seen in the synthesis of intermediate 55 starting from Benzothiazole-6-carboxylic acid. The overall yield was 38%.

HPLC: 1.92 min. LC-MS: M/Z ESI: 0.97 min, 164.27 (M+1). ¹H NMR (DMSO-d6) δ 10.1 (s, 1H), 9.60 (s, 1H), 8.60 (s, 1H), 8.20 (m, 1H), 8.10 (d, 1H).

### Intermediate 57: 3-Methyl-benzofuran-5-carbaldehyde

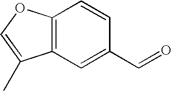

This intermediate was accessed through the same route as intermediate 1 using Ethyl-2-acetyl-4-bromophenoxy acetate as starting material. Overall yield 50%.

LC-MS: M/Z ESI: 1.55 min, 161.34 (M+1). ¹H NMR (DMSO-d6) δ 10.1 (s, 1H), 8.21 (d, J=1.5 Hz 1H), 7.92 (d, J=1.3 Hz, 1H), 7.88-7.84(dd, J=1.6 Hz, 1H), 7.73-7.71 (d, J=8.5 Hz, 1H), 2.25 (s, 3H).

### Intermediate 58: 3-Bromo-benzofuran-5-carbaldehyde

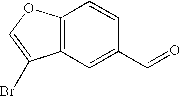

### Step I: 2,3-Dibromo-2,3-dihydro-benzofuran-5-carbaldehyde

Intermediate 1 (2 g, 13.7 mmol) was dissolved in 10 ml CHCl3 and cooled to −10° C. To this was added a solution of Br₂ in CHCl₃ (1.55 eq., c=4.162 mol/l). The reaction mixture turned dark and was allowed to reach r.t. during 1 h. HPLC indicated complete addition of bromine. The solvent and remaining bromine were evaporated under reduced pressure affording a reddish oil (4.1 g=90%), which was used for the next step without further purification.

HPLC: 3.43 min

### Step II: 3-Bromo-1-benzofuran-5-carbaldehyde

To a solution of 2,3-dibromo-2,3-dihydro-1-benzofuran-5-carbaldehyde (4.1 g) in dry ethanol (15 mL) was added a solution of KOH (2.2 eq.) in dry ethanol (14 mL) and refluxed at 70° C. for 1 h. The reaction mixture was cooled, diluted with water and extracted with EtOAc (3×50 mL). The organic layer was washed with water, brine and dried. The solvent was removed under vacuum and the residue was purified by flash chromatography (pet. ether/EtOAc 99.5:0.5) to give the title compound as a pale yellow solid (2.91 g (80% pure), yield=78%).

HPLC: 3.35 min. ¹H NMR (DMSO-d₆, 300 MHz) δ 10.12 (s, 1H), 8.47 (s, 1H), 8.14 (d, J=1.5 Hz, 1H), 7.97 (dd, J=8.6, 1.5 Hz, 1H), 7.87 (d, J=8.6 Hz, 1H).

### Intermediate 59: 3-Phenylethynyl-benzofuran-5-carbaldehyde

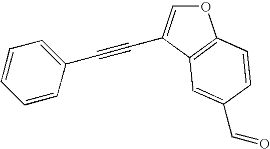

In a dry flask 3-Bromo-1-benzofuran-5-carbaldehyde (1 g, 4.4 mmol) were dissolved in anhydrous THF (50 ml). To this was added under Argon Bis(triphenylphosphine) palladium(II) chloride (160 mg, 0.2 mmol), TEA (2.81 mL, 5 eq.), CuI (40 mg, 0.2 mmol) and Phenylacetylene (897 mg, 8.8 mmol). The reaction was heated at 55° C. for 2 days. The crude was filtered through celite and purified on silica gel using as eluent cyclohexan-ethyl acetate (7-3) affording 680 mg (yield: 56%).

HPLC: 4.71 min. ¹H NMR (DMSO-d6) δ 10.14 (s, 1H), 8.64 (s, 1H), 8.38 (s, 1H), 7.97 (dd, J=1.5 Hz, 8.3 Hz, 1H), 7.90 (d, J=8.6 Hz, 1H), 7.65 (m, 2H), 7.46 (m, 3H).

### Intermediate 60: 3-(5-Formyl-benzofuran-3-yl)-acrylic acid ethyl ester

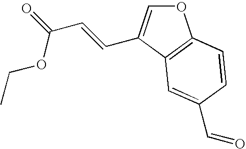

In a sealed tube 3-Bromo-1-benzofuran-5-carbaldehyde (500 mg, 2.22 mmol) was dissolved in 7 ml of ACN. To this solution was added PPh₃ (1.16 g, 4.44 mmol), Pd(II)acetate (500 mg, 2.2 mmol), Et3N (073 mL, 5.55 mmol) and finally acrylic acid ethyl ester (2.41 ml, 22 mmol). The tube was sealed and the reaction was heated at 120° C. for one hour. The crude was filtered on celite to eliminate inorganic contaminations. The solvents were evaporated and the crude was purified by silica gel chromatography using cyclohexane-AcOEt 95-5 to 50-50. A pale yellow solid was obtained (400 mg, yield: 42%).

HPLC: 3.69 min. ¹H NMR (DMSO-d6) δ 10.15 (s, 1H), 8.70 (s, 2H), 7.97 (d, J=9 Hz 1H), 7.88 (s, 1H), 7.82 (s, 1H), 6.76 (d, J=15 Hz, 1H), 4.23 (q, J=6 Hz, 12 Hz, 2H), 1.28 (t, J=9 Hz, 3H).

### Intermediate 61: 2,3-Dihydro-benzo[1,4]oxazine-4,6-dicarboxylic acid 4-tert-butyl ester 6-methyl ester

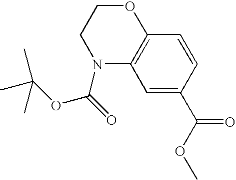

### Step 1: 3-Amino-4-hydroxy-benzoic acid methyl ester

To a 2000 ml three-necked flask containing 3-Nitro-4-hydroxy-benzoic acid methyl ester (43 g, 218 mmol) in MeOH (860 ml; 20 vols) was added palladium on carbon in water (2 g in 10 ml of water). Ammonium formate (68.76 g, 5 eq.) was added in a single portion under stirring. After 2 to 3 minutes a suspension was observed, and temperature rised from 20° C. to 30° C. Ice bath was used to cool reaction mixture to 20° C. and the reaction was stirred at 20° C. for 40 minutes until completion (no more yellow color). Reaction mixture was filtered on silica plug, rinsed with MeOH, and the filtrate was concentrated under vacuum to give a green oil which was taken up in ethyl acetate (400 ml). The organic phase was washed twice with water, dried over MgSO₄, filtered and concentrated to give a cream solid m=31.35 g (86%).

LC-MS: M/Z ESI: 0.81 min, 168.37 (M+1)

### Step II: 3,4-Dihydro-2H-benzo[1,4]oxazine-6-carboxylic acid methyl ester*hydrochloride

To a 2000 ml three-necked flask under N₂ containing 3-Amino-4-hydroxy-benzoic acid methyl ester (31.35 g, 187 mmol) in anhydrous DMF (630 ml; 20 vols) at RT, was added K₂CO₃ (103 g, 4 eq.) in one portion followed by 1,2dibromoethane (65 ml, 4 eq.) in one portion. The reaction mixture was stirred at 70° C. for 12 h. Temperature was allowed to cool down to RT, and HCl1N was added until pH=8, and extraction was performed using diethyl ether (3*200 ml). The organic phase was washed with water (2*200 ml) and dried over MgSO4 and concentrated to afford a brown red oil with solid, which was taken up again in diethyl ether (450 ml) and THF (50 ml) and filtered to remove a white solid. To the filtrate was added HCl1N, and diethyl ether (130 ml) was added, suspension was stirred at RT for 5 minutes and filtered to give 27.6 g of crude product. The aqueous phases were again extracted with ethyl acetate to afford additional 6.23 g of product. The combined fractions (32 g) were recrystallised from EtOH (420 ml; 13 vols) to give after filtration and drying a white powder (19.47 g (16.37 g free base)), yield=40%.

HPLC: 1.954 min. LC-MS: M/Z ESI: 1.27 min, 194.45 (M+1).

### Step III: 2,3-Dihydro-benzo[1,4]oxazine-4,6-dicarboxylic acid 4-tert-butyl ester 6-methyl ester

To a 500 ml three-necked flask containing 3,4-Dihydro-2H-benzo[1,4]oxazine-6-carboxylic acid methyl ester*hydrochloride in suspension in THF (145 ml; 10 vols) under N₂, DIEA (27 ml, 2.5 eq.) was added in one portion at RT and partial solubilisation was observed. Boc andydride/(16.4 g, 1.2 eq.) was added in one portion and the reaction was stirred at 65° C. for 5 days. During that time several small portions of 0.2 eq. of Boc₂O and DIEA were added. THF was removed under vacuum and the residue was taken up in DCM 150 ml The organic phase was washed with a saturated solution of NaHCO₃ and then with brine. After drying over MgSO₄ and filtration, volatiles were removed under vacuum and the residue was recristallised from EtOH (80 ml) to give cream crystals (14.8 g, 76%).

HPLC: 4.038 min. ¹H NMR (CDCl3) δ 8.49 (s, 1H), 7.68 (dd, J=3 Hz, 9 Hz, 1H), 6.89 (d, J=9 Hz, 1H), 4.30 (q, J=3 Hz, 9 Hz, 2H), 3.89 (m, 5H), 1.62 (s, 9H).

### Intermediate 62: 6-Formyl-2,3-dihydro-benzo[1,4]oxazine-4-carboxylic acid tert-butyl ester

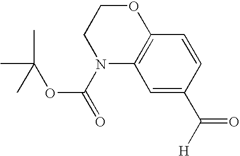

This intermediate was accessed through oxido-reduction as described for intermediate 5.

HPLC: 3.727 min. LC-MS: M/Z ESI: 1.81 min, 264.34 (M+1). ¹H NMR (DMSO-d6) δ 9.83 (s, 1H), 8.35 (s, 1H), 7.53 (d, J=6 Hz, 1H), 7.05 (d, J=9 Hz, 1H), 4.31 (t, J=3 Hz, 2H), 3.83 (t, J=6 Hz, 2H), 1.50 (s, 9H).

### Intermediate 63: 6-Formyl-benzo[1,4]oxazine-4-carboxylic acid tert-butyl ester

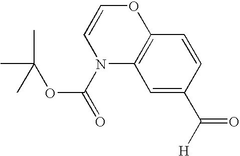

### Step I: 2,3-Dibromo-2,3-dihydro-benzo[1,4]oxazine-4,6-dicarboxylic acid 4-tert-butyl ester 6-methyl ester

To a solution of 2,3-Dihydro-benzo[1,4]oxazine-4,6-dicarboxylic acid 4-tert-butyl ester 6-methyl ester (500 mg, 1.7 mmol) in dry carbon tetrachloride (20 ml) was added N-Bromosuccinimide (667 mg, 3.75 mmol) and a catalytic amount of benzoylperoxide. The resulting mixture was stirred and heated with a bulp lamp (100 W) at reflux for 45 min. The mixture was allowed to cool and the succinimide was filtered off. The filtrate was evaporated to yield an oil (767 mg, 99%) sufficiently pure to be used for the next step.

HPLC: 3.978 min

### Step II: Benzo[1,4]oxazine-4,6-dicarboxylic acid 4-tert-butyl ester 6-methyl ester

2,3-Dibromo-2,3-dihydro-benzo[1,4]oxazine-4,6-dicarboxylic acid 4-tert-butyl ester 6-methyl ester (767 mg, 1.7 mmol) from proceeding step was stirred in acetone (14 ml) at RT for 2 h with NaI (1.27 g, 8.5 mmol). The solvent was removed, EtOAc, water and 1 M sodium thiosulfate were added. After separating phases the organic layer was washed with brine. The solvent was concentrated and the crude was purified on silica gel using CH/EtOAc 7:3 to obtain a colorless oil (456 mg, 92%).

HPLC: 4.386 min.

### Step III: 6-Hydroxylmethyl-benzo[1,4]oxazine-4-carboxylic acid tert-butyl ester

### Step IV: 6-Formyl-benzo[1,4]oxazine-4-carboxylic acid tert-butyl ester

Step III and IV were carried out according to the synthesis of intermediate 5.

HPLC: 3.388 min.

### Intermediate 64: (6-Formyl-3-oxo-2,3-dihydro-benzo[1,4]oxazin-4-yl)-acetic acid methyl ester

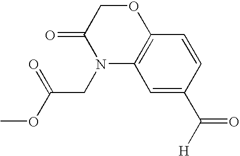

### Step I: Methyl-3-amino-4-hydroxybenzoate

To a solution of 3-amino-4-hydroxybenzoic acid (10 g, 0.65 mol) in methanol (1.5 L) was added thionylchloride (233 g, 1.96 mol) drop-wise at 5-10° C. with stirring and allowed to reflux at 65° C. for 16 h. Excess methanol and thionylchloride was distilled off and crude dissolved in ethylacetate (500 mL). The organic layer was washed with 5% aqueous NaHCO₃ solution, water, brine and dried. The solvent was removed under vacuum to give methyl-3-amino-4-hydroxybenzoate (105 g, 95%).

### Step II: Methyl-3-oxo-3,4-dihydro-2H-1,4-benzoxazin-6-carboxylate

To a mixture of methyl-3-amino-4-hydroxybenzoate (105 g, 0.62 mol) and benzyltriethylammonium chloride (142 g, 0.62 mol) in dry CHCl₃ (1.5 L) was added NaHCO₃ (211 g, 2.5 mol) with stirring. The reaction mixture was cooled to −5° C., added chloroacetylchloride (85 g, 0.75 mol) in dry CHCl₃ (350 mL) over a period of 1.5 h at the same temperature. The reaction mixture was then heated to 55° C. for 16 h. The solvent was removed under vacuum, added water (3 L) and filtered off the solid. The solid product was dried and recrystallised from ethanol to give methyl-3-oxo-3,4-dihydro-2H-1,4-benzoxazin-6-carboxylate (108 g, 83%).

### Step III: 6-(Hydroxymethyl)-2H-1,4-benzoxazin-3(4H)-one

A solution of methyl-3-oxo-3,4-dihydro-2H-1,4-benzoxazin-6-carboxylate (30 g, 0.145 mol) in dry CH₂Cl₂ (500 mL) was cooled to −78° C. and added DIBAL-H (51 g, 0.36 mol) over a period of 45 min and then stirred at the same temperature for 14 h. The reaction mixture was quenched with 1.5N HCl and filtered off the solid product. The solid compound was dried under vacuum to give 6-(hydroxymethyl)-2H-1,4-benzoxazin-3(4H)-one (18 g, 69%).

### Step IV: TBDMS-6-(hydroxymethyl)-2H-1,4-benzoxazin-3(4H)-one

To a solution of 6-(hydroxymethyl)-2H-1,4-benzoxazin-3(4H)-one (18 g, 0.1 omol) in dry DMF (250 mL) was added imidazole (13.7 g, 0.2 mol) and stirred at 0° C. for 30 min. To the above reaction mixture was added TBDMSiCl (23 g, 0.15 mol) in portions and stirred at RT for 4 h. The reaction mixture was diluted with water and filtered off the solid obtained. The solid was dried under vacuum to give TBDMS-6-(hydroxymethyl)2H-1,4-benzoxazin-3(4H)-one (24.5 g, 83%).

### Step V: Methyl-[6-(hydroxymethyl)-3-oxo-2,3-dihydro-4H-1,4-benzoxazin-4-yl]acetate

To a suspension of NaH (0.3 g, 0.01 mol) in dry DMF (15 mL) was added TBDMS-6-(hydroxymethyl)2H-1,4-benzoxazin-3(4H)-one (2 g, 0.0068 mol) at 0° C. with stirring and allowed to stir at RT for 2 h. The reaction mixture was cooled to 0° C., added methylchloroacetate (1 g, 0.0088 mol) and stirred at RT for 12 h. The reaction mixture was further cooled to 0° C., added 50 mL of 1.5N HCl solution and stirred at RT for 12 h. The reaction mixture was diluted with water (200 mL), extracted with ethylacetate (3×150 mL). The combined organic layer was washed with 10% aqueous NaHCO₃ solution, brine and dried. The solvent was removed under vacuum and crude purified by column chromatography over silica gel (CHCl₃/Methanol, 99.5:0.5) to give methyl-[6-(hydroxymethyl)-3-oxo-2,3-dihydro-4H-1,4-benzoxazin-4-yl]acetate (1.2 g, 70%).

### Step VI: Methyl-[6-(Formyl)-3-oxo-2,3-dihydro-4H-1,4-benzoxazin-4-yl]acetate

A mixture of PCC (4.2 g, 0.019 mol) and celite (4 g) in dry CH₂Cl₂ (100 mL) was cooled to 0° C. and slowly added a solution of methyl-[6-(hydroxymethyl)-3-oxo-2,3-dihydro-4H-1,4-benzoxazin-4-yl]acetate (1.2 g, 0.0048 mol) in CH₂Cl₂ (30 mL) under N₂. The reaction mixture was stirred at RT for 2 h, passed through celite, washed with CH₂Cl₂ (50 mL) and concentrated to give crude product, which was purified on silica gel affording 1.05 g (87%).

LC-MS: M/Z ESI: 1.15 min, 250.41 (M+1). ¹HNMR (DMSO-d6) δ 9.88 (s, 1H), 7.65-7.60 (m, 2H), 7.24 (d, J=8.1 Hz, 1H), 4.85 (d, J=9.9 Hz, 4H), 3.71 (s, 3H).

### Intermediate 65: 4-Butyl-3-oxo-3,4-dihydro-2H-benzo[1,4]oxazine-6-carbaldehyde

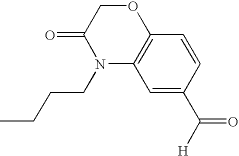

This intermediate was synthesized according to the synthesis of intermediate 2. Overall yield 33%.

LC-MS: M/Z ESI: 1.60 min, 234.35 (M+¹). ¹H NMR (DMSO-d6) δ 7.66 (d, J=0.7 Hz, 1H), 7.58 (dd, J=1.7 Hz, 8.1 Hz, 1H), 7.18 (d, J=8.2 Hz, 1H), 4.77 (s, 2H), 3.96 (t, J=7.3 Hz, 1H), 1.61-1.51 (m, 3H), 1.97-1.27 (m, 3H), 0.91 (t, J=7.3 Hz, 3H).

### Intermediate 66: 4-Benzyl-3-oxo-3,4-dihydro-2H-benzo[1,4]oxazine-6-carbaldehyde

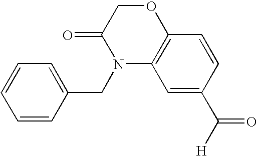

This intermediate was synthesized according to the synthesis of intermediate 2. Overall yield 29%.

¹H NMR (DMSO-d6) δ 9.78 (s, 1H), 7.58 (dd, J=1.5 Hz, 7.9 Hz, 1H), 7.47 (d, J=1.9 Hz, 7.40-7.18 (m, 6H), 5.22 (s, 2H), 4.95 (s, 2H), 3.3 (d, J=7.2 Hz, 1H).

### Intermediate 67: 2-Chloro-5-[1,3]dioxolan-2-yl-benzofuran

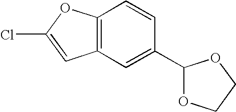

### Step I: 5-[1,3]Dioxolan-2-yl-benzofuran

A mixture of benzofuran-5-carbaldehyde (150 mg, 1.03 mmol), ethylene glycol (230 ul, 4 eq), trimethyl orthoformate (123 ul, 1.1 eq) and tetrabutylammonium tribromide (49 mg, 0.1 eq) was stirred at room temperature for one night. Some starting material could be detected by TLC. However, the reaction mixture was poured into saturated NaHCO₃ solution and the product was extracted with ethyl acetate. Combined organic layers were dried over anhydrous sodium sulfate, filtrated and concentrated to give a crude product, which was purified by flash chromatography using cyclohexane/ethyl acetate 20:0.75 as solvents. The title compounds was obtained in 36% yield (70 mg).

LC-MS: M/Z ESI: 1.51 min, 191.30 (M+1).

### Step II: 2-Chloro-5-[1,3]dioxolan-2-yl-benzofuran

5-[1,3]Dioxolan-2-yl-benzofuran (50 mg, 0.26 mmol) was dissolved in THF (2 mL) and the solution was cooled down to −78° C. Butyl lithium (180 uL, 1.1 eq.) was added dropwise. This mixture was stirred 30 min at 25° C. Then the reaction mixture was cooled down to −78° C. and NCS (39 mg, 1.1 eq.) dissolved in 1 mL THF was added dropwise to the reaction mixture. After 1 h30 at −78° C. only small amount of starting material could be detected. The temperature was increased slowly to room temperature overnight. Water and ethyl acetate were added to the mixture and the aqueous layer was extracted 3 times. Combined organic phases was dried over MgSO₄, filtrated and evaporated to give 2-Chloro-5-[1,3]dioxolan-2-yl-benzofuran (48.1 mg, 81%) sufficiently pure to be used in the next step.

LC-MS: M/Z ESI: 1.77 min, 225.23 (M+1).

### Intermediate 68: 3-Amino-benzo[d]isoxazole-5-carbaldehyde

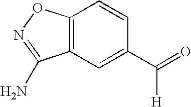

Kaiser oxime resin (Novabiochem 01-64-0188) (250 mg) was washed with DCM and THF (3 times 5 min), 2 ml of THF was added followed by the addition of 300 ul of potassium-tert.butoxide (1M in THF, 1.2 eq.) at r.t. The resin turned orange and was shaken in the Quest210™ for 15′. 2-Fluoro-5-formyl-benzonitrile (75 mg, 2 eq.) in 1 ml THF was added and the reaction was heated at 55° C. for 12 h. The resin was washed with DCM, MeOH, water (each 2×5 minutes) and MeOH (4×5 min). The resin was dried at 40° C. with a flow of Argon for 30′ before cleaving.

The so dried resin was treated with TFA/5N HCl 4:1 (2.5 ml) for 2 h at 55° C. The solution was collected in 20 ml vials and the resin was washed twice with 4 ml of DCM. The collected fractions were evaporated with the Genevac HT4 to dryness affording: 37 mg (92%) of pure 3-Amino-benzo[d]isoxazole-5-carbaldehyde.

HPLC: 1.47 min. LC-MS: M/Z ESI: 0.82 min, 163.26 (M+1).

### Intermediate 69: 4-Piperidin-1-yl-quinazoline-6-carboxylic acid methyl ester

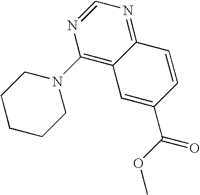

This intermediate was prepared according to the synthesis of intermediate 8 starting from 4-Chloro-quinazoline-6-carboxylic acid methyl ester (intermediate 7).

HPLC: 1.81 min. LC-MS: M/Z ESI: 1.78 min, 272.32(M+1).

### Intermediate 70: 4-Piperidin-1-yl-quinazoline-6-carbaldehyde

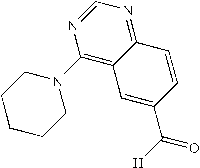

This intermediate was prepared according to the synthesis of intermediate 5 starting from 4-Piperidine-quinazoline-6-carboxylic acid methyl ester (intermediate 71).

HPLC: 1.36 min. LC-MS: M/Z ESI: 1.40 min, 242.32(M+1).

### Intermediate 71: 3-(5-Formyl-benzofuran-3-yl)-propionic acid ethyl ester

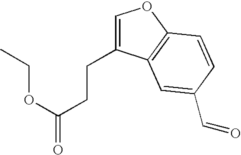

100 mg of 3-(5-Formyl-benzofuran-3-yl)-acrylic acid ethyl ester (intermediate 62) were dissolved in EtOAc in the presence of Palladium on charcoal and Argon. To this was connected a H₂-balloon and hydrogenation was carried out for 12 h. The palladium was filtered off and the solvents were evaporated affording pure title compound (80 mg, 80%).

HPLC: 3.53 min. LC-MS: M/Z ESI: 1.68 min, 247.25 (M+1).

### Intermediate 72: 2-Methyl-5-[1,3]dioxolan-2-yl-benzofuran

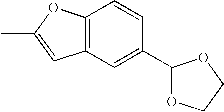

5-[1,3]Dioxolan-2-yl-benzofuran (50 mg, 0.26 mmol) was dissolved in THF (2 mL) and the solution was cooled down to −78° C. Butyl lithium (180 uL, 1.1 eq.) was added dropwise. This mixture was stirred 30 min at 25° C. Then the reaction mixture was cooled down to −78° C. and iodomethane (18.1 uL, 1.1 eq.) dissolved in 1 mL THF was added dropwise to the reaction mixture. The temperature was increased slowly to room temperature overnight. Despite some starting material was detected, water and ethyl acetate were added to the mixture and the aqueous layer was extracted 3 times. Combined organic phases was dried over MgSO₄, filtrated and evaporated to give 2-methyl-5-[1,3]dioxolan-2-yl-benzofuran (41.2 mg, 70%) sufficiently pure to be used in the next step.

LC-MS: M/Z ESI: 1.71 min, 205.34 (M+1).

### Intermediate 73: 5-[1,3]Dioxolan-2-yl-benzofuran-2-carboxylic acid methyl ester

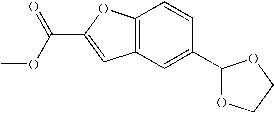

5-[1,3]Dioxolan-2-yl-benzofuran (50 mg, 0.26 mmol) was dissolved in THF (2 mL) and the solution was cooled down to −78° C. Butyl lithium (180 uL, 1.1 eq.) was added dropwise. This mixture was stirred 30 min at 25° C. Then the reaction mixture was cooled down to −78° C. and methyl cyanoformate (23 uL, 1.1 eq.) dissolved in 1 mL THF was added dropwise to the reaction mixture. After 1 h30 only small amount of starting material was detected and two major compounds were formed (expected product/dimer 73:27). The temperature was increased slowly to room temperature overnight. Water and ethyl acetate were added to the mixture and the aqueous layer was extracted 3 times. Combined organic phases was dried over MgSO₄, filtrated and evaporated to give the 5-[1,3]Dioxolan-2-yl-benzofuran-2-carboxylic acid methyl ester (31.9 mg, 44%) mixed with the dimer (expected product/dimer 46:54). The mixture was used directly in the next step.

LC-MS: M/Z ESI: 1.54 min, 249.26 (M+1) and 1.88 min, 407.20 (M+1, Dimer).

### Intermediate 74: 3-Bromo-2-fluoro-benzofuran-5-carbaldehyde

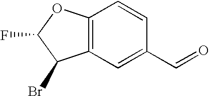

Benzofuran-5-carbaldehyde (100 mg, 0.68 mmol) in ether (1 mL) was added to a cold solution (−78° C.) of NBS (158 mg, 1.3 eq) and pyridinium poly(hydrogen fluoride) 70% (0.850 mL) in ether (4 mL) in a polypropylene tube. The reaction was allowed to warm up to room temperature overnight. The reaction mixture was poured into ice water and extracted with ether. The ether phase was washed with aqueous bicarbonate, dried over sodium sulfate, filtrated and evaporated to give 3-bromo-2-fluoro-benzofuran-5-carbaldehyde (141.6 mg). It was purified on reverse phase HPLC (solvents gradient H₂O/CH₃CN 0.1% TFA) affording the title compound (62 mg, 37%), which was used in the next step.

LC-MS: M/Z ESI: 1.56 min. HPLC=3.11 min (99.34%). ¹H NMR: (DMSO-d6) δ9.94 (s, 1H), 8.09 (d, 1H, ³J=1.8 Hz), 7.99 (dd, 1H, ³J=8.4, 1.8 Hz), 7.38 (d, 1H, ³J=8.4 Hz), 6.87 (d, 1H, ²J_{H-F}=59 Hz), 6.01 (d, 1H, ³J_{H-F}=15.1 Hz). ¹⁹F NMR: (DMSO-d6) δ−114.80, −114.88.

### Intermediate 75: 2-Fluoro-5-[1,3]dioxolan-2-yl-benzofuran

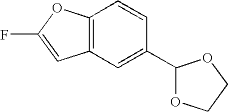

5-[1,3]Dioxolan-2-yl-benzofuran (50 mg, 0.26 mmol) was dissolved in THF (2 mL) and the solution was cooled down to −78° C. Butyl lithium (180 uL, 1.1 eq.) was added dropwise. This mixture was stirred 30 min at 25° C. Then the reaction mixture was cooled down to −78° C. and N-fluorodibenzenesulfonamide (91 mg, 1.1 eq.), dissolved in 1 mL THF, was added dropwise to the reaction mixture. The mixture was stirred overnight between −78° C. and room temperature. Water and ethyl acetate were added to the mixture and the aqueous layer was extracted 3 times. Combined organic phases was dried over MgSO₄, filtrated and evaporated, to give the 2-Fluoro-5-[1,3]dioxolan-2-yl-benzofuran (75 mg) mixed with side products. However it was sufficiently pure to be used for the next step.

The following examples have been synthesized:

### EXAMPLE 1

### Preparation of 5-(1,3-benzodioxol-5-ylmethylene)-1,3-thiazolidine-2,4-dione

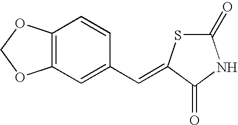

In a 100 ml round bottom flask were placed 20 g of thiazolidine, 15.6 g of piperonal and 7.7 g of beta-alanine in 80 ml of acetic acid. The reaction was stirred for 3 h at 100° C. and then slowly cooled to room temperature, while the desired condensation product crystallized. The crystals were filtered, washed with acetic acid (rt.) and water than recrystallized from DME (25 ml), affording 28 g (84%) of pure 5-(1,3-benzodioxol-5-ylmethylene)-1,3-thiazolidine-2,4-dione. The corresponding potassium salt was obtained via the following route: 5-(1,3-benzodioxol-5-ylmethylene)-1,3-thiazolidine-2,4-dione was suspended in THF, followed by the addition of 1N solution of KOH in water (1.0 eq.). A clear solution has been obtained, which upon lyophilization gave pure potassium salt of 5-(1,3-benzodioxol-5-ylmethylene)-1,3-thiazolidine-2,4-dione.

HPLC: 3.48 min. LC-MS: M/Z ESI: 1.31 min, 248.12 (M−1). NMR (parent): ¹H NMR (DMSO-d6) δ12.5 (br. s, 1H), 7.71 (s, 1H), 7.06-7.16 (m, 3H), 6.12 (s, 2H).

In cases were the final compounds did not crystallize from the reaction solutions, small quantities of water were added, leading to the precipitation of the desired condensation product.

The crude either precipitated pure enough from the reaction mixture, or was recrystallized from an appropriate solvent like DME, methanol, EtOAc or purified by flash-chromatography using EtOAc, cyclohexane mixtures as eluents.

Alternativly the final compounds could be synthesized in a parallel manner according to the following protocol:

In a parallel synthesizer Quest 210™ was placed the corresponding aldehyde, to which was added a mixture of piperidine (17.9 mg/tube) and 2,4-thiazolidinedione (49.2 mg/tube) in DME (2 ml/tube). The reactions were stirred for 3 h at 120° C. and then cooled to room temperature under agitation. 2 ml of H₂O were added. Those compounds, which precipitated were filtered off via the lower manifold. The remaining clear solutions were reduced in volume, followed by the addition of water. The so formed solids were filtered and washed with little amount of DME, affording pure condensation products.

### EXAMPLE 2

### Preparation of 5-(1,3-benzodioxol-5-ylmethylene)-2-thioxo-1,3-thiazolidin-4-one

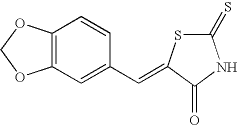

In a 24 ml vial was placed 1 g of commercially available rhodanine, 1.3 g of piperonal and 0.5 ml of TEA in 10 ml of DME. The reaction was stirred for 5 h at 120° C. and then cooled to room temperature upon which the final product precipitated. The solid was filtered and washed with DME affording 1.6 g (80%) of orange powder.

LC-MS: M/Z ESI: 1.46 min, 266.00 (M+1), 264.08 (M−1). NMR (parent): ¹H NMR (DMSO-d6) δ13.75 (br. s, 1H), 7.58 (s, 1H), 7.08-7.18 (m, 3H), 6.14 (s, 2H).

### EXAMPLE 3

### Preparation of 5-(2,3-dihydro-1,4-benzodioxin-6-ylmethylene)-1,3-thiazolidine-2,4-dione

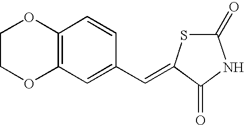

Following the general method as outlined in Example 1, starting from 2,3-dihydro-1,4-benzodioxin-6-carbaldehyde and 1,3-thiazolidine-2,4-dione, the title compound was obtained.

HPLC: 2.58 min. LC-MS: M/Z ESI: 1.32 min, 262.16 (M−1). ¹H NMR: (DMSO-d6) δ 12.52 (br. s, 1H), 7.68 (s, 1H,), 7.09 (dd, 2H, J=1.9, 7.1), 7.00 (d, 1H, J=9.0 Hz), 4.36-4.22 (m, 4H).

### EXAMPLE 4

### Preparation of 5-(2,3-dihydro-1-benzofuran-5-ylmethylene)-1,3-thiazolidine-2,4-dione

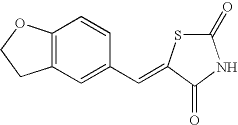

Following the general method as outlined in Example 1, starting from 2,3-dihydro-1-benzofuran-5-carbaldehyde and 1,3-thiazolidine-2,4-dione, the title compound was obtained.

HPLC: 3.27 min. LC-MS: M/Z ESI: 1.37 min. 246.18 (M−1). ¹HNMR: (DMSO-d6) δ9.80 (br. s, 1H), 7.37 (s, 1H,), 7.25 (d, 1H, J=8.3), 7.21 (s, 1H), 6.80 (d, 1H, J=8.3 Hz), 4.54 (t, 2H, J=8.85), 3.19 (t, 2H, J=8.85)

### EXAMPLE 5

### Preparation of 5-[(7-methoxy 1,3-benzodioxol-5-yl)methylene]-1,3-thiazolidine-2,4-dione

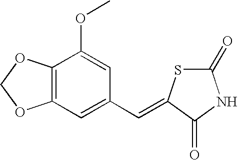

Following the general method as outlined in Example 1, starting from 7-methoxy-1,3-benzodioxol-5-yl)carbaldehyde and 1,3-thiazolidine-2,4-dione, the title compound was obtained.

HPLC: 3.57 min. LC-MS: M/Z ESI: 1.30 min, 278.07 (M−1). ¹H NMR: (DMSO-d6) δ 12.63 (br. s, 1H), 7.78 (s, 1H,), 7.65 (s, 1H), 7.57 (d, 1H, J=8.5 Hz), 7.45 (dd, 2H, J=0.8, 7.6).

### EXAMPLE 6

### Preparation of 5-[(9,10-dioxo-9,10-dihydroanthracen-2-yl)methylene]-1,3-thiazolidine-2,4-dione

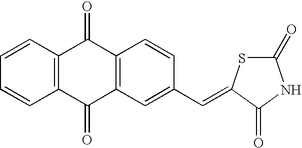

Following the general method as outlined in Example 1, starting from (9,10-dioxo-9,10-dihydroanthracen-2-yl)carbaldehyde and 1,3-thiazolidine-2,4-dione, the title compound was obtained.

HPLC: 4.12 min. LC-MS: M/Z ESI: 1.50 min, 334.09 (M−1).

### EXAMPLE 7

### Preparation of (5-[(2,2-difluoro-1,3-benzodioxol-5-yl)methylene]-1,3-thiazolidine-2,4-dione

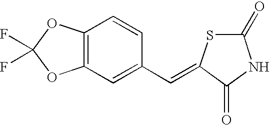

Following the general method as outlined in Example 1, starting from f2,2-difluoro-1,3-benzodioxol-5-yl)carbaldehyde and 1,3-thiazolidine-2,4-dione, the title compound was obtained.

HPLC: 3.85 min. LC-MS (10 min.): M/Z ESI: 3.15 min, 284.11 (M−1). ¹HNMR: (DMSO-d6) δ12.63 (br. s, 1H), 7.78 (s, 1H,), 7.65 (s, 1H), 7.57 (d, 1H, J=8.5 Hz), 7.45 (dd, 2H, J=0.8, 7.6)

### EXAMPLE 8

### Preparation of 5-(1,3-dihydro-2-benzofuran-5-ylmethylene)-1,3-thiazolidine-2,4-dione

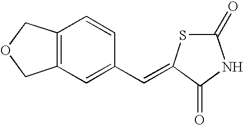

Following the general method as outlined in Example 1, starting from 1,3-dihydro-2-benzofuran-5-carbaldehyde (intermediate 4) and 1,3-thiazolidine-2,4-dione, the title compound was obtained.

HPLC: 2.89 min. LC-MS: M/Z ESI: 1.20 min, 246.20 (M−1). ¹H NMR: (DMSO-d6) δ 12.60 (br. s, 1H), 7.80 (s, 1H,), 7.56-7.42 (m, 2H), 5.03 (s, 4H)

### EXAMPLE 9

### Preparation of 5-(1-benzofuran-5-ylmethylene)-1,3-thiazolidine-2,4-dione

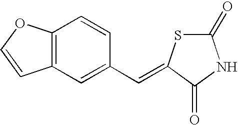

Following the general method as outlined in Example 1, starting from 1-benzofuran-5-carbaldehyde (intermediate 1) and 1,3-thiazolidine-2,4-dione, the title compound was obtained.

HPLC: 3.54 min. LC-MS: M/Z ESI: 1.47 min., 244.20 (M−1). ¹H NMR: (DMSO-d6) δ 12.58 (br. s, 1H), 8.10 (d, 1H, J=2.2 Hz), 7.92 (s, 2H), 7.74 (d, 1H, J=8.6 Hz), 7.57 (d, 1H, J=8.6 Hz), 7.07 (s, 1H)

### EXAMPLE 10

### Preparation of 5-[(4-methyl-3-oxo-3,4-dihydro-2H-1,4-benzoxazin-6-yl)methylene]-1,3-thiazolidine-2,4-dione

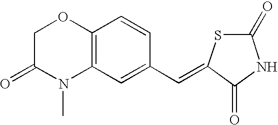

Following the general method as outlined in Example 1, starting from [(4-methyl-3-oxo-3,4-dihydro-2H-1,4-benzoxazin-6-yl)carbaldehyde (intermediate 2) and 1,3-thiazolidine-2,4-dione, the title compound was obtained.

HPLC: 2.79 min. LC-MS: M/Z ESI: 1.19 min, 289.22 (M−1). ¹H NMR: (DMSO-d6) δ 12.58 (br. s, 1H), 7.81 (s, 1H), 7.41 (s, 1H), 7.13-7.26 (d, 2H), 4.74 (s, 2H), 2.99 (s, 3H)

### EXAMPLE 11

### Preparation of 5-(1,3-benzodioxol-5-ylmethylene)-2-imino-1,3-thiazolidine-4-one

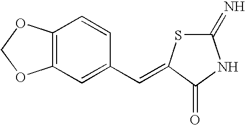

Following the general method as outlined in Example 1, starting from 1,3-benzodioxol-5-carbaldehyde and 2-imino-1,3-thiazolidin-4-one, the title compound was obtained.

HPLC: 2.29 min. LC-MS: M/Z ESI: 1.21 min, 247.25 (M−1).

### EXAMPLE 12

### Preparation of 5-Quinolin-6-ylmethylene-thiazolidine-2,4-dione

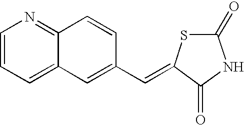

Following the general method as outlined in Example 1, starting from quinoline-6-carbaldehyde (intermediate 5) and 1,3-thiazolidine-2,4-dione, the title compound was obtained.

HPLC: 1.445 min. LC-MS: M/Z ESI: 1.17 min, 257.21 (M+1). ¹H NMR: (DMSO-d6) δ 8.88 (d, J=6 Hz, 1H), 8.40 (d, J=9 Hz, 1H), 8.07-7.90 (m, 3H), 7.55 (q, J=6 Hz, 9 Hz, 1H), 7.45 (s, 1H).

### EXAMPLE 13

### 5-Quinolin-6-ylmethylene-2-thioxo-thiazolidin-4-one

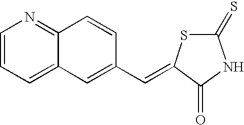

Following the general method as outlined in Example 1, starting from quinoline-6-carbaldehyde (intermediate 5) and rhodanine, the title compound was obtained.

HPLC: 2.05 min. LC-MS: M/Z ESI: 1.25 min, 273.14 (M+1). ¹H NMR: (DMSO-d6) δ 14.00 (br. s, 1H), 8.97 (d, J=2.3 Hz, 1H), 8.23 (d, J=9 Hz, 1H), 8.10 (d, J=9 Hz, 1H), 7.95 (d, J=9 Hz, 1H), 7.79 (s, 1H), 7.61 (q, J=3 Hz, 9 Hz, 1H).

### EXAMPLE 14

### 2-Imino-5-quinolin-6-ylmethylene-thiazolidin-4-one

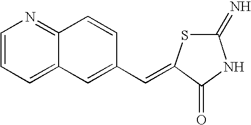

Following the general method as outlined in Example 1, starting from quinoline-6-carbaldehyde (intermediate 5) and 2-imino-1,3-thiazolidin-4-one, the title compound was obtained.

HPLC: 1.16 min. LC-MS: M/Z ESI: 1.10 min, 256.18 (M+1). ¹H NMR: (DMSO-d6) δ 12.58 (br. s, 1H), 8.84 (s, 1H), 8.37 (d, J=6 Hz, 1H), 8.02-7.86 (m, 3H), 7.52 (q, J=6 Hz, 9 Hz, 1H), 7.26 (s, 1H), 7.02 (b. s, 1H).

### EXAMPLE 15

### 5-(3-Methyl-benzo[d]isoxazol-5-ylmethylene)-thiazolidine-2,4-dione

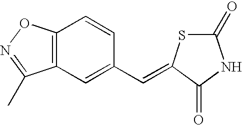

Following the general method as outlined in Example 1, starting from 3-Methyl-benzo[d]isoxazole-5-carbaldehyde (intermediate 6) and 1,3-thiazolidine-2,4-dione, the title compound was obtained.

HPLC: 2.99 min. LC-MS: M/Z ESI: 1.30 min, 259.17 (M−1). ¹H NMR: (DMSO-d6) δ 12.58 (br. s, 1H), 8.08 (s, 1H), 7.95 (s, 1H), 7.85 (s, 2H), 2.59 (s, 3H).

### EXAMPLE 16

### 5-(4-Phenyl-quinazolin-6-ylmethylene)-thiazolidine-2,4-dione

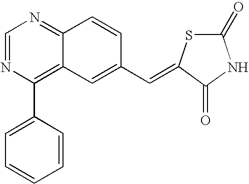

Following the general method as outlined in Example 1, starting from 4-Phenyl-quinazoline-6-carbaldehyde (intermediate 13) and 1,3-thiazolidine-2,4-dione, the title compound was obtained.

HPLC: 3.45 min. LC-MS: M/Z ESI: 1.25 min, 334.15 (M+1). ¹H NMR: (DMSO-d6) δ 12.74 (br. s, 1H), 9.43 (s, 1H), 8.24 (m, 2H), 8.00-7.86 (m, 2H), 7.72-7.66 (m, 5H).

### EXAMPLE 17

### 5-(4-Dimethylamino-quinazolin-6-ylmethylene)-thiazolidine-2,4-dione

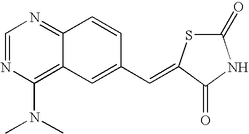

Following the general method as outlined in Example 1, starting from 4-Dimethylamino-quinazoline-6-carbaldehyde (intermediate 14) and 1,3-thiazolidine-2,4-dione, the title compound was obtained.

HPLC: 1.47 min. LC-MS: M/Z ESI: 1.26 min, 301.26 (M+1). ¹H NMR: (DMSO-d6) δ 8.81 (s, 1H), 8.54 (s, 1H), 8.16-7.95 (m, 3H), 7.13-7.26 (d, 2H), 3.63 (s, 6H).

The following examples were synthesized as desribed in Example 1 and 17 starting from intermediates 15 to 31 and 1,3-thiazolidine-2,4-dione

| | Intermediate# | | |
|---|---|---|---|
| | as starting | | |
| Example | material | Compound name | Mass (M + 1 ) |
| 18 | 16 | 5-[(4-aminoquinazolin-6-yl)methylene]-1,3- | 273.29 |
| | | thiazolidine-2,4-dione | |
| 19 | 15 | 5-[(4-piperidin-1-ylquinazolin-6-yl)methylene]-1,3- | 341.40 |
| | | thiazolidine-2,4-dione | |
| 20 | 22 | 5-[(4-morpholin-4-ylquinazolin-6-yl)methylene]- | 343.20 |
| | | 1,3-thiazolidine-2,4-dione | |
| 21 | 17 | 5-{[4-(benzylamino)quinazolin-6-yl]methylene}- | 363.10 |
| | | 1,3-thiazolidine-2,4-dione | |
| 22 | 21 | 5-{[4-(diethylamino)quinazolin-6-yl]methylene}- | 329.30 |
| | | 1,3-thiazolidine-2,4-dione | |
| 23 | 18 | 5-({4-[(pyridin-2-ylmethyl)amino]quinazolin-6- | 364.40 |
| | | yl}methylene)-1,3-thiazolidine-2,4-dione | |
| 24 | 19 | 5-({4-[(pyridin-3-ylmethyl)amino]quinazolin-6- | 364.40 |
| | | yl}methylene)-1,3-thiazolidine-2,4-dione | |
| 25 | 23 | ethyl 1-{6-[(2,4-dioxo-1,3-thiazolidin-5- | 413.20 |
| | | ylidene)methyl]quinazolin-4-yl}piperidine-3- | |
| | | carboxylate | |
| 26 | 25 | ethyl 1-{6-[(2,4-dioxo-1,3-thiazolidin-5- | 413.30 |
| | | ylidene)methyl]quinazolin-4-yl}piperidine-4- | |
| | | carboxylate | |
| 27 | 24 | tert-butyl 1-{6-[(2,4-dioxo-1,3-thiazolidin-5- | 427.20 |
| | | ylidene)methyl]quinazolin-4-yl}-L-prolinate | |
| 28 | 20 | 5-{[4-(4-methylpiperazin-1-yl)quinazolin-6- | 356.13 |
| | | yl]methylene}-1,3-thiazolidine-2,4-dione | |
| 29 | 31 | 5-{[4-(4-pyrimidin-2-ylpiperazin-1-yl)quinazolin- | 420.20 |
| | | 6-yl]methylene}-1,3-thiazolidine-2,4-dione | |
| 30 | 30 | 5-({4-[4-(4-fluorophenyl)piperidin-1-yl]quinazolin- | 435.30 |
| | | 6-yl}methylene)-1,3-thiazolidine-2,4-dione | |
| 31 | 29 | 5-{[4-(4-benzylpiperidin-1-yl)quinazolin-6- | 431.30 |
| | | yl]methylene}-1,3-thiazolidine-2,4-dione | |
| 32 | 28 | 5-({4-[4-(2-phenylethyl)piperidin-1-yl]quinazolin- | 445.40 |
| | | 6-yl}methylene)-1,3-thiazolidine-2,4-dione | |
| 33 | 27 | 5-{[4-(4-methylpiperidin-1-yl)quinazolin-6- | 355.20 |
| | | yl]methylene}-1,3-thiazolidine-2,4-dione | |
| 34 | 26 | 5-{[4-(4-hydroxypiperidin-1-yl)quinazolin-6- | 357.40 |
| | | yl]methylene}-1,3-thiazolidine-2,4-dione | |

### EXAMPLE 35

### 1-[6-(2,4-Dioxo-thiazolidin-5-ylidenemethyl)-quinazolin-4-yl]-piperidine-4-carboxylic acid

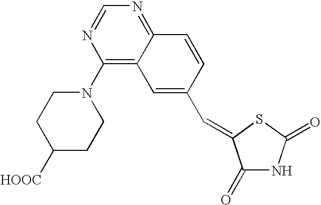

50 mg of Ethyl 1-{6-[(2,4-dioxo-1,3-thiazolidin-5-ylidene)methyl]quinazolin-4-yl}piperidine-4-carboxylate (example 26) was dissolved in 2 ml solution of THF/water (1/1). A few drops of 5N NaOH were added, and the reaction was stirred for 12 h at rt. After completion of the reaction, solvents were evaporated and titled compound was precipitated in diethylether as a yellow solid (40 mg, 82%).

HPLC: 1.43 min. LC-MS: M/Z ESI: 1.15 min, 385.20 (M+1).

### EXAMPLE 36

### 1-[6-(2,4-Dioxo-thiazolidin-5-ylidenemethyl)-quinazolin-4-yl]-piperidine-3-carboxylic acid

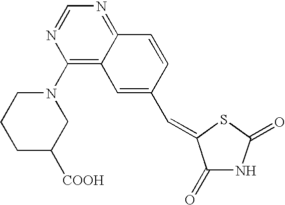

Following the general method as outlined in Example 35, the title compound was obtained.

HPLC: 1.50 min. LC-MS: M/Z ESI: 1.10 min, 385.40 (M+1).

### EXAMPLE 37

### 1-[6-(2,4-Dioxo-thiazolidin-5-ylidenemethyl)-quinazolin-4-yl]-pyrrolidine-2-carboxylic acid

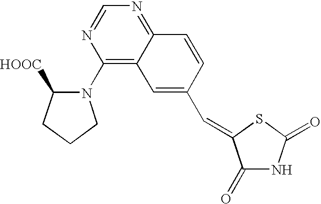

10 mg of tert-butyl 1-{6-[(2,4-dioxo-1,3-thiazolidin-5-ylidene)methyl]quinazolin-4-yl}-L-prolinate (example 27) was stirred in a 25% (TFA/DCM) solution for 12 h at rt. The solvents were evaporated under vacuo and expected compound was precipitated with dietyl ether to give pure 1-[6-(2,4-Dioxo-thiazolidin-5-ylidenemethyl)-quinazolin-4-yl]-pyrrolidine-2-carboxylic acid (7 mg, 81%).

HPLC: 1.43 min. LC-MS: M/Z ESI: 1.10 min, 371.30 (M+1).

### EXAMPLE 38

### 5-(4-Methylamino-quinazolin-6-ylmethylene)-thiazolidine-2,4-dione

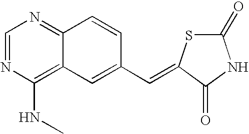

Following the general method as outlined in Example 1, starting from 4-methylamino-quinazoline-6-carbaldehyde (intermediate 11) and 1,3-thiazolidine-2,4-dione, the title compound was obtained.

HPLC: 1.43 min. LC-MS: M/Z ESI: 1.03 min, 287.19 (M+1). ¹H NMR: (DMSO-d6) δ 11.97 (br. s, 1H), 8.53 (br. s, 2H), 8.37 (s, 1H), 7.92 (d, J=8 Hz, 1H), 7.76 (s, 2H), 3.03 (s, 3H)

### EXAMPLE 39

### 5-(4-Methoxy-quinazolin-6-ylmethylene)-thiazolidine-2,4-dione

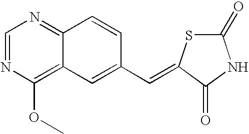

Following the general method as outlined in Example 1, starting from 4-methoxy-quinazoline-6-carbaldehyde (intermediate 10) and 1,3-thiazolidine-2,4-dione, the title compound was obtained.

HPLC: 2.57 min. LC-MS: M/Z ESI: 1.12 min, 288.20 (M+1). ¹H NMR: (DMSO-d6) δ 12.74 (br. s, 1H), 8.86 (s, 1H), 8.32 (s, 1H), 8.11 (m, 1H), 8.03-7.98 (m, 2H), 4.18 (s, 3H)

### EXAMPLE 40

### 2-Imino-5-(4-methylamino-quinazolin-6-ylmethylene)-thiazolidin-4-one

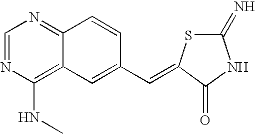

Following the general method as outlined in Example 1, starting from 4-methylamino-quinazoline-6-carbaldehyde (intermediate 11) and 2-imino-1,3-thiazolidin-4-one, the title to compound was obtained.

HPLC: 2.43 min. LC-MS: M/Z ESI: 1.07 min, 286.14 (M+1).

### EXAMPLE 41

### 2-Imino-5-(4-piperidine-quinazolin-6-ylmethylene)-thiazolidin-4-one

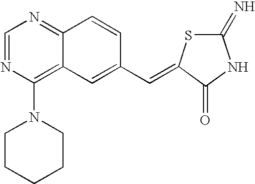

Following the general method as outlined in Example 1, starting from 4-piperidine-quinazoline-6-carbaldehyde (intermediate 72) and 2-imino-1,3-thiazolidin-4-one, the title compound was obtained.

HPLC: 1.78 min. LC-MS: M/Z ESI: 1.40 min, 340.26 (M+1). ¹H NMR: (DMSO-d6) δ 8.76 (s, 1H), 8.18 (s, 1H), 8.16 (d, J=6 Hz, 1H), 7.88 (d, J=9 Hz, 1H), 7.80 (s, 1H), 4.09 (s, 4H), 1.80 (s, 6H).

### EXAMPLE 42

### 2-Imino-5-(4-dimethylamino-quinazolin-6-ylmethylene)-thiazolidin-4-one

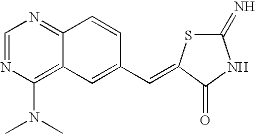

Following the general method as outlined in Example 1, starting from 4-piperidine-quinazoline-6-carbaldehyde (intermediate 14) and 2-imino-1,3-thiazolidin-4-one, the title compound was obtained.

HPLC: 1.32 min. LC-MS(10 min.): M/Z ESI: 1.54 min, 300.23 (M+1). ¹HNMR: (DMSO-d6) δ8.82 (s, 1H), 8.53 (s, 1H), 8.16 (d, J=9 Hz, 1H), 7.87 (t, J=9 Hz, 2H), 3.65 (s, 6H).

### EXAMPLE 43

### 5-(2-Methyl-2H-benzotriazol-5-ylmethylene)-thiazolidine-2,4-dione

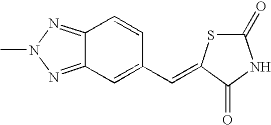

Following the general method as outlined in Example 1, starting from 2-Methyl-2H-benzotrizaole-5-carbaldehyde (intermediate 33) and thiazolidindione, the title compound was obtained.

HPLC: 2.68 min. ¹H NMR: (DMSO-d6) δ12.58 (br. s, 1H), 7.98 (s, 1H), 7.92 (d, J=9 Hz, 1H), 7.62 (d, J=6 Hz, 1H), 7.43 (s, 1H), 4.48 (s, 3H).

### EXAMPLE 44

### 5-(3-Methyl-3H-benzotriazol-5-ylmethylene)-thiazolidine-2,4-dione

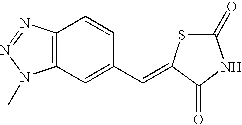

Following the general method as outlined in Example 1, starting from 3-Methyl-3H-benzotrizaole-5-carbaldehyde (intermediate 34) and thiazolidindione, the title compound was obtained.

HPLC: 2.35 min. LC-MS: M/Z ESI: 1.22 min, 259.23 (M−1). ¹H NMR: (DMSO-d6) δ 12.58 (br. s, 1H), 8.17 (d, J=9 Hz, 1H), 8.07 (s, 1H), 7.62 (d, J=6 Hz, 1H), 7.47 (s, 1H), 4.33 (s, 3H).

### EXAMPLE 45

### 5-(3-Ethyl-3H-benzimidazol-5-ylmethylene)-thiazolidine-2,4-dione

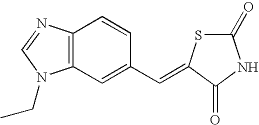

5-(4-Amino-3-ethylamino-benzylidene)-thiazolidine-2,4-dione (50 mg, 0.19 mmol) (intermediate 36) was dissolved in formic acid (5 mL) and the solution stirred at 100° C. over night. Formic acid was then removed in vacuo. The crude residue was then purified by silica gel column to give the title compound (35 mg, 63%).

HPLC: 1.71 min. LC-MS: M/Z ESI: 0.82 min, 274.21 (M+1).

The following examples were synthesized as desribed in Example 45 starting from intermediates 37 to 54 and 1,3-thiazolidine-2,4-dione.

| | Intermediate# | | |
|---|---|---|---|
| | as starting | | |
| Example | material | Compound name | Mass (M + 1) |
| 46 | 37 | 5-{[1-(4-phenylbutyl)-1H-benzimidazol-6- | 378.30 |
| | | yl]methylene}-1,3-thiazolidine-2,4-dione | |
| 47 | 50 | 5-[(1-prop-2-yn-1-yl-1H-benzimidazol-6- | 284.24 |
| | | yl)methylene]-1,3-thiazolidine-2,4-dione | |
| 48 | 38 | 5-[(1-{2-[4-(trifluoromethyl)phenyl]ethyl}-1H- | 418.17 |
| | | benzimidazol-6-yl)methylene]-1,3-thiazolidine-2,4- | |
| | | dione | |
| 49 | 39 | 5-({1-[2-(4-hydroxyphenyl)ethyl]-1H-benzimidazol- | 366.26 |
| | | 6-yl}methylene)-1,3-thiazolidine-2,4-dione | |
| 50 | 40 | methyl 4-{6-[(2,4-dioxo-1,3-thiazolidin-5- | 386.35 |
| | | ylidene)methyl]-1H-benzimidazol-1- | |
| | | yl}cyclohexanecarboxylate | |
| 51 | 41 | 5-({1-[2-(5-methoxy-1H-indol-3-yl)ethyl]-1H- | 419.21 |
| | | benzimidazol-6-yl}methylene)-1,3-thiazolidine-2,4- | |
| | | dione | |
| 52 | 42 | 5-({1-[(1-methyl-1H-pyrazol-4-yl)methyl]-1H- | 340.99 |
| | | benzimidazol-6-yl}methylene)-1,3-thiazolidine-2,4- | |
| | | dione | |
| 53 | 43 | 5-({1-[2-(3,4-dimethoxyphenyl)ethyl]-1H- | 410.37 |
| | | benzimidazol-6-yl}methylene)-1,3-thiazolidine-2,4- | |
| | | dione | |
| 54 | 54 | 5-({1-[2-(4-phenoxyphenyl)ethyl]-1H- | 442.51 |
| | | benzimidazol-6-yl}methylene)-1,3-thiazolidine-2,4- | |
| | | dione | |
| 55 | 44 | 5-({1-[4-(trifluoromethyl)benzyl]-1H-benzimidazol- | 404.16 |
| | | 6-yl}methylene)-1,3-thiazolidine-2,4-dione | |
| 56 | 45 | 4-{6-[(2,4-dioxo-1,3-thiazolidin-5-ylidene)methyl]- | 372.18 |
| | | 1H-benzimidazol-1-yl}cyclohexanecarboxylic acid | |
| 57 | 46 | 5-[(1-isobutyl-1H-benzimidazol-6-yl)methylene]- | 302.25 |
| | | 1,3-thiazolidine-2,4-dione | |
| 58 | 47 | 5-({1-[2-(1,3-benzodioxol-4-yl)ethyl]-1H- | 394.27 |
| | | benzimidazol-6-yl}methylene)-1,3-thiazolidine-2,4- | |
| | | dione | |
| 59 | 48 | 5-({1-[2-(2-phenoxyphenyl)ethyl]-1H- | 442.29 |
| | | benzimidazol-6-yl}methylene)-1,3-thiazolidine-2,4- | |
| | | dione | |
| 60 | 49 | 5-{[1-(3,3-diphenylpropyl)-1H-benzimidazol-6- | 440.27 |
| | | yl]methylene}-1,3-thiazolidine-2,4-dione | |
| 61 | 51 | 5-{[1-(2-methoxybenzyl)-1H-benzimidazol-6- | 366.33 |
| | | yl]methylene}-1,3-thiazolidine-2,4-dione | |
| 62 | 52 | 5-{[1-(3-furylmethyl)-1H-benzimidazol-6- | 326.24 |
| | | yl]methylene}-1,3-thiazolidine-2,4-dione | |
| 63 | 53 | 5-[(1-propyl-1H-benzimidazol-6-yl)methylene]-1,3- | 288.18 |
| | | thiazolidine-2,4-dione | |

### EXAMPLE 64

### 5-Quinoxalin-6-ylmethylene-thiazolidine-2,4-dione

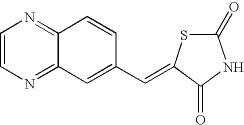

Following the general method as outlined in Example 1, starting from quinoxaline-6-carbaldehyde (intermediate 55) and thiazolidindione, the title compound was obtained.

HPLC: 2.48 min. LC-MS: M/Z ESI: 1.01 min, 256.20 (M−1). ¹H NMR: (DMSO-d6) δ 12.58 (br. s, 1H), 8.93 (d, J=9 Hz, 2H), 8.18 (s, 1H), 8.10 (d, J=9 Hz, 1H), 8.03 (d, J=9 Hz, 1H), 7.51 (s, 1H).

### EXAMPLE 65

### 5-Quinoxalin-6-ylmethylene-2-thioxo-thiazolidin-4-one

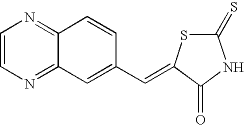

Following the general method as outlined in Example 1, starting from quinoxaline-6-carbaldehyde (intermediate 55) and rhodanine, the title compound was obtained.

HPLC: 3.10 min. LC-MS: M/Z ESI: 1.17 min, 272.13 (M−1). ¹H NMR: (DMSO-d6) δ 12.00 (br. s, 1H), 9.02 (s, 2H), 8.31 (s, 1H), 8.21 (d, J=9 Hz, 1H), 8.04 (d, f-9 Hz, 1H), 7.90 (s, 1H)

### EXAMPLE 66

### 2-Imino-5-quinoxalin-6-ylmethylene-thiazolidin-4-one

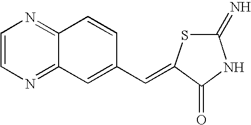

Following the general method as outlined in Example 1, starting from quinoxaline-6-carbaldehyde (intermediate 55) and 2-imino-1,3-thiazolidin-4-one, the title compound was obtained.

HPLC: 1.97 min. LC-MS: M/Z ESI: 1.02 min, 255.19 (M−1). ¹H NMR: (DMSO-d6) δ 9.57-9.30 (b. d, J=81 Hz, 2H), 9.00 (s, 2H), 8.26-8.07 (m, 3H), 7.84 (s, 1H).

### EXAMPLE 67

### 5-Benzothiazol-6-ylmethylene-thiazolidine-2,4-dione

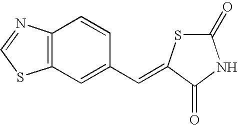

Following the general method as outlined in Example 1, starting from quinoxaline-6-carbaldehyde (intermediate 56) and thiazolidindione, the title compound was obtained.

HPLC: 2.85 min. LC-MS: M/Z ESI: 1.06 min, 261.11 (M−1). ¹HNMR: (DMSO-d6) δ 12.58 (br. s, 1H), 9.39 (s, 1H), 8.27 (s, 1H), 8.11 (d, J=9 Hz, 1H), 7.70 (d, J=9 Hz, 1H), 7.42 (s, 1H).

### EXAMPLE 68

### 5-(3-Methyl-benzofuran-5-yl]ethylene)-thiazolidine-2,4-dione

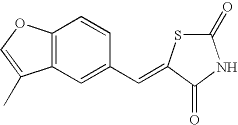

Following the general method as outlined in Example 1, starting from 3-Methyl-benzofuran-5-carbaldehyde (intermediate 57) and 1,3-thiazolidine-2,4-dione, the title compound was obtained.

HPLC: 1.47 min. LC-MS: M/Z ESI: 1.15 min, 257.21 (M−1). ¹H NMR: (DMSO-d6) δ 12.50 (br. s, 1H), 8.87 (d, J=6 Hz, 1H), 8.38 (d, J=9 Hz, 1H), 8.07 (t, J=12 Hz, 2H), 7.92 (d, J=9 Hz, 1H), 7.53 (q, J=6 Hz, 12 Hz, 1H), 7.45 (s, 1H).

### EXAMPLE 69

### 5-(2-Bromo-3-methyl-benzofuran-5-ylmethylene)-thiazolidine-2,4-dione

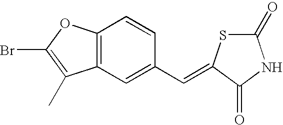

In a 25 ml 3 neck flask was placed 5-(3-methyl-benzofuran-5-ylmethylene)-thiazolidine-2,4-dione (100 mg, 0.39 mmol) (example 68) and Br2 (20 ul, 1 eq.) in 2 ml of AcOH at 0° C. The mixture was allowed to warm to room temperature. After 2 h at room temperature another equivalent of Br₂ was added. After 3 h the reaction was filtered off to obtain a yellow product being the title compound (87 mg, 66%).

LC-MS: M/Z ESI: 1.69 min, 339.8 (M+1). ¹H NMR: (DMSO-d6) δ12.50 (br. s, 1H), 7.93 (s, 1H), 7.82 (s, 1H), 7.72 (d, J=6 Hz, 1H), 7.54 (d, J=6 Hz, 1H), 2.20 (s, 3H).

### EXAMPLE 70

### 5-(3-bromo-benzofuran-5-ylmethylene)-thiazolidine-2,4-dione

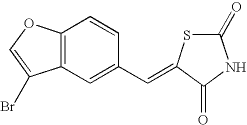

Following the general method as outlined in Example 1, starting from 3-Bromo-benzofuran-5-carbaldehyde (intermediate 58) and 1,3-thiazolidine-2,4-dione, the title compound was obtained.

HPLC: 3.92 min. LC-MS: M/Z ESI: 1.57 min, 325.17 (M+1). ¹H NMR: (DMSO-d6) δ 12.60 (br. s, 1H), 8.42 (s, 1H), 8.00 (s, 1H), 7.85 (d, J=23 Hz, 1H), 7.76 (s, 1H), 7.63 (d, J=23 Hz, 1H).

### EXAMPLE 71

### 3-[5-(2,4-Dioxo-thiazolidin-5-ylidenemethyl)-benzofuran-3-yl]-acrylic acid ethyl ester

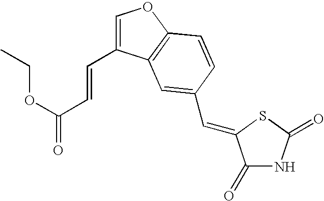

Following the general method as outlined in Example 1, starting from 3-(5-Formyl-benzofuran-3-yl)-acrylic acid ethyl ester (intermediate 60) and 1,3-thiazolidine-2,4-dione, the title compound was obtained.

HPLC: 4.00 min. LC-MS: M/Z ESI: 1.60 min, 342.20 (M−1). ¹H NMR: (DMSO-d6) δ 12.50 (br. s, 1H), 8.63 (s, 1H), 8.42 (s, 1H), 8.08 (s, 1H), 7.83 (m, 2H), 7.62 (s, 1H), 4.22 (q, J=6 Hz, 9 Hz, 2H), 1.28 (t, J=9 Hz, 3H).

### EXAMPLE 72

### 3-[5-(2,4-Dioxo-thiazolidin-5-ylidenemethyl)-benzofuran-3-yl]-acrylic acid

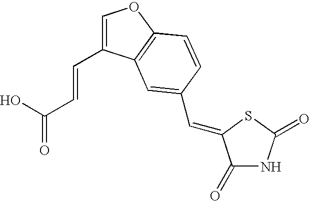

3-[5-(2,4-Dioxo-thiazolidin-5-ylidenemethyl)-benzofuran-3-yl]-acrylic acid ethyl ester (205 mg, 0.6 mmol) (example 71) were dissolved in THF/water 4:2. To this solution was added under stirring 81 mg (4 eq.) of LiOH.H₂O. The reaction was stirred for 15 h. The solvents were evaporated, and the residue was precipitated with ether. The solid was washed with 1N HCl and dried to afford 170 mg (90%) of pure 3-[5-(2,4-Dioxo-thiazolidin-5-ylidenemethyl)-benzofuran-3-yl]-acrylic acid.

HPLC: 3.25 min. LC-MS: M/Z ESI: 1.01 min, 314.11 (M−1). ¹H NMR: (DMSO-d6) δ8.22 (s, 1H), 8.03 (s, 1H), 7.58 (dd, J=9 Hz, 33 Hz, 2H), 7.43 (s, 1H), 7.25 (d, J=18 Hz, 1H), 7.07 (s, 1H).

### EXAMPLE 73

### 5-[3-(3-Oxo-3-piperidin-1-yl-propenyl)-benzofuran-5-ylmethylene]-thiazolidine-2,4-dione

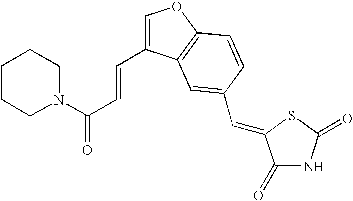

180 mg (0.57 mmol) of 3-[5-(2,4-Dioxo-thiazolidin-5-ylidenemethyl)-benzofuran-3-yl]-acrylic acid (example 72) was suspended in THF (25 ml). To this suspension was added DIEA (2 eq.) and piperidine (3 eq.). Under stirring was added PyBOP (1.5 eq.). After 30 min the reaction mixture became clear, after an additional 1 h a precipitate was formed. The reaction was stirred overnight. The precipitate was filtered off and washed with THF and 1N HCl affording the title compound in high purity.

HPLC: 3.91 min. LC-MS: M/Z ESI: 1.58 min, 383.22 (M+1). ¹H NMR: (DMSO-d6) δ 8.46 (s, 1H), 8.19 (s, 1H), 7.71-7.51 (m, 4H), 7.23 (d, J=15 Hz, 1H), 3.73 (d, J=48 Hz, 2H), 1.51 (d, J=36 Hz, 3H).

The following amides were synthesized according to the synthesis of example 73.

| | Amine as starting | | |
|---|---|---|---|
| Example | material | Compound name | Mass (M + 1) |
| 74 | Proline- | Methyl 1-((3-{5-[(2,4-dioxo-1,3-thiazolidin-5- | 427.15 |
| | methylester | ylidene)methyl]-1-benzofuran-3-yl}prop-2- | |
| | | enoyl)prolinate | |
| 75 | D-proline- | Methyl 1-((3-{5-[(2,4-dioxo-1,3-thiazolidin-5- | 413.15 |
| | methylester | ylidene)methyl]-1-benzofuran-3-yl}prop-2- | |
| | | enoyl)-D-prolinate | |
| 76 | Pyrollidine | (5-({3-[(3-oxo-3-pyrrolidin-1-ylprop-1-en-1- | 369.52 |
| | | yl]-1-benzofuran-5-yl}methylene)-1,3- | |
| | | thiazolidine-2,4-dione | |
| 77 | Morpholine | 5-({3-[3-morpholin-4-yl-3-oxoprop-1-en-1-yl]- | 385.07 |
| | | 1-benzofuran-5-yl}methylene)-1,3-thiazolidine- | |
| | | 2,4-dione | |
| 78 | L-proline- | Methyl 1-(3-{5-[(2,4-dioxo-1,3-thiazolidin-5- | 427.13 |
| | methylester | ylidene)methyl]-1-benzofuran-3-yl}prop-2- | |
| | | enoyl)-L-prolinate | |
| 79 | N-methyl- | N-cyclohexyl-3-{5-[(2,4-dioxo-1,3-thiazolidin- | 411.12 |
| | cyclohexylamine | 5-ylidene)methyl]-1-benzofuran-3-yl}-N- | |
| | | methylacrylamide | |
| 80 | N-ethyl- | 3-{5-[(2,4-dioxo-1,3-thiazolidin-5- | 387.10 |
| | hydroxyethyl- | ylidene)methyl]-1-benzofuran-3-yl}-N-ethyl-N- | |
| | amine | (2-hydroxyethyl)acrylamide | |
| 81 | Cyclobutylamine | N-cyclobutyl-3-{5-[(2,4-dioxo-1,3-thiazolidin- | 369.13 |
| | | 5-ylidene)methyl]-1-benzofuran-3- | |
| | | yl}acrylamide | |
| 82 | Azetidine | 5-({3-[3-azetidin-1-yl-3-oxoprop-1-en-1-yl]-1- | 355.64 |
| | | benzofuran-5-yl}methylene)-1,3-thiazolidine- | |
| | | 2,4-dione | |
| 83 | 1,3-dihydro-2H- | 5-({3-[3-(1,3-dihydro-2H-isoindol-2-yl)-3- | 415.00 |
| | isoindole | oxoprop-1-en-1-yl]-1-benzofuran-5- | (M − 1) |
| | | yl}methylene)-1,3-thiazolidine-2,4-dione | |
| 84 | Azepan | 5-({3-[3-azepan-1-yl-3-oxoprop-1-en-1-yl]-1- | 397.46 |
| | | benzofuran-5-yl}methylene)-1,3-thiazolidine- | |
| | | 2,4-dione | |
| 85 | Piperidin-1- | 3-{5-[(2,4-dioxo-1,3-thiazolidin-5- | 398.00 |
| | ylamine | ylidene)methyl]-1-benzofuran-3-yl}-N- | |
| | | piperidin-1-ylacrylamide | |
| 86 | Pyridin-3-yl- | 3-{5-[(2,4-dioxo-1,3-thiazolidin-5- | 406.10 |
| | methylamine | ylidene)methyl]-1-benzofuran-3-yl}-N- | |
| | | (pyridin-3-ylmethyl)acrylamide | |
| 87 | Cyclohexylamine | N-cyclohexyl-3-{5-[(2,4-dioxo-1,3-thiazolidin- | 397.08 |
| | | 5-ylidene)methyl]-1-benzofuran-3- | |
| | | yl}acrylamide | |
| 88 | 4-N-methyl- | 5-({3-[3-(4-methylpiperazin-1-yl)-3-oxoprop-1- | 398.02 |
| | piperazine | en-1-yl]-1-benzofuran-5-yl}methylene)-1,3- | |
| | | thiazolidine-2,4-dione | |
| 89 | Cycloheptylamine | N-cycloheptyl-3-{5-[(2,4-dioxo-1,3-thiazolidin- | 411.44 |
| | | 5-ylidene)methyl]-1-benzofuran-3- | |
| | | yl}acrylamide | |
| 90 | Pyroline | 5-({3-[3-(2,5-dihydro-1H-pyrrol-1-yl)-3- | 367.11 |
| | | oxoprop-1-en-1-yl]-1-benzofuran-5- | |
| | | yl}methylene)-1,3-thiazolidine-2,4-dione | |
| 91 | Cyclopentylamine | N-cyclopentyl-3-{5-[(2,4-dioxo-1,3-thiazolidin- | 383.11 |
| | | 5-ylidene)methyl]-1-benzofuran-3- | |
| | | yl}acrylamide | |

### EXAMPLE 92

### 3-[5-(2,4-Dioxo-thiazolidin-5-ylidenemethyl)-benzofuran-3-yl]-propionic acid ethyl ester

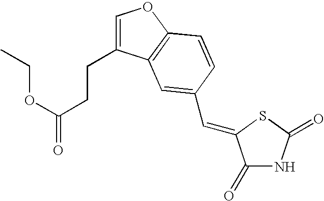

Following the general method as outlined in Example 1, starting from 3-(5-Formyl-benzofuran-3-yl)-propionic acid ethylester (intermediate 71) and 1,3-thiazolidine-2,4-dione, the title compound was obtained.

HPLC: 3.94 mn. LC-MS: M/Z ESI: 2.87 min, 346.15 (M+1). ¹H NMR: (DMSO-d6) δ12.58 (br. s, 1H), 7.92 (d, J=6 Hz, 3H), 7.72 (d, J=9 Hz, 1H), 7.53 (d, J=9 Hz, 1H), 4.03 (q, J=9 Hz, 15 Hz, 2H), 2.94 (t, J=9 Hz, 2H), 2.73 (t, J=6 Hz, 2H), 1.14 (t, J=6 Hz).

### EXAMPLE 93

### 3-[5-(2,4-Dioxo-thiazolidin-5-ylidenemethyl)-benzofuran-3-yl]-propionic acid

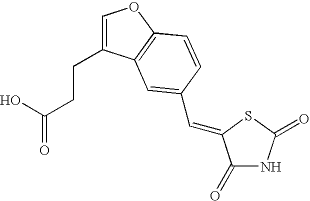

The title compound was obtained applying standard saponifications techniques as described for example 72 using example 92 as starting material.

HPLC: 3.09 min. LC-MS(10 min.): M/Z ESI: 1.19 min, 316.14 (M−1). ¹H NMR: (DMSO-d6) δ12.58 (br. s, 1H), 12.22 (b. s, 1H), 7.93 (d, J=12 Hz, 3H), 7.70 (d, f-9 Hz, 1H), 7.54 (d, J=9 Hz, 1H), 2.91 (t, J=9 Hz, 2H), 2.65 (t, 6 Hz, 2H).

### EXAMPLE 94

### 5-[3-(3-Oxo-3-piperidin-1-yl-propyl)-benzofuran-5-ylmethylene]-thiazolidine-2,4-dione

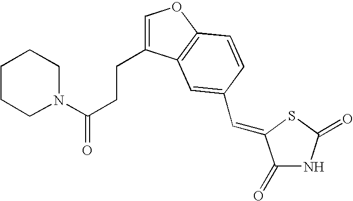

The title compound was obtained applying the synthetic protocol as described for example 73 using example 93 as starting material.

HPLC: 3.783 min. LC-MS: M/Z ESI: 1.46 min, 385.14 (M+1). ¹H NMR: (DMSO-d6) δ 12.66 (br. s, 1H), 8.06 (s, 3H), 8.01 (s, 1H), 7.79 (s, 1H), 3.50-1.60 (m, 14H).

### EXAMPLE 95

### 6-(2,4-Dioxo-thiazolidin-5-ylidenemethyl)-2,3-dihydro-benzo[1,4]oxazine-4-carboxylic acid tert-butyl ester

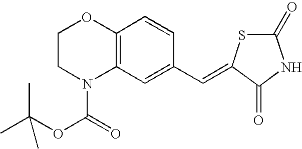

Following the general method as outlined in Example 1, starting from 6-Formyl-2,3-dihydro-benzo[1,4]oxazine-4-carboxylic acid tert-butyl ester (intermediate 62) and 1,3-o thiazolidine-2,4-dione, the title compound was obtained.

HPLC: 2.52 min. LC-MS: M/Z ESI: min, 261.21 (M−Boc−1).

### EXAMPLE 96

### 5-(3,4-Dihydro-2H-benzo[1,4]oxazin-6-ylmethylene)-thiazolidine-2,4-dione

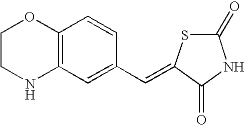

100 mg of 6-Formyl-2,3-dihydro-benzo[1,4]oxazine-4-carboxylic acid tert-butyl ester (intermediate 62) were treated with TFA/DCM 25% for 2 h. The solvents were evaporated to dryness and the remaining crude was used for the Knoevenagel reaction as outlined in Example 1 without further purification to obtain the title compound as yellow solid.

HPLC: 2.56 min. LC-MS: M/Z ESI: 1.14 min, 261.24 (M−1). ¹H NMR: (DMSO-d6) δ 12.58 (br. s, 1H), 7.57 (s, 1H), 6.78 (s, 3H), 4.17 (t, J=3 Hz, 2H), 3.28 (t, J=6 Hz, 2H).

### EXAMPLE 97

### 5-(4-Benzoyl-3,4-dihydro-2H-benzo[1,4]oxazin-6-ylmethylene)-thiazolidine-2,4-dione

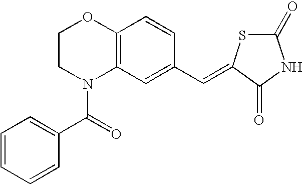

5-(3,4-Dihydro-2H-benzo[1,4]oxazin-6-ylmethylene)-thiazolidine-2,4-dione (example 96) (35 mg, 0.13 mmol) in 4 ml anhydrous THF were treated with benzoylchloride (156 uL, 10 eq.) in the presence of DIEA (2 eq.) for 3 h. Excess of benzoylchloride was hydrolysed, EtOAc was added and the organic phase was washed with NaHCO₃ and brine. The crude was purified on silica gel using EtOAc/cyclohexane 3:7 as eluent affording 14 mg (35%) of the title compound.

HPLC: 4.57 min. LC-MS: M/Z ESI: 2.11 min, 364.91 (M−1).

The following example was synthesized in the same way as described for example 97.

### EXAMPLE 98

### 5-(4-Acetyl-3,4-dihydro-2H-benzo[1,4]oxazin-6-ylmethylene)-thiazolidine-2,4-dione

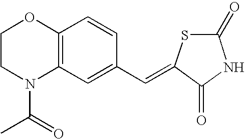

Yield=43 mg (95%)

HPLC: 2.65 min. LC-MS: M/Z ESI: 1.12 min, 305.24 (M+1). ¹H NMR: (DMSO-d6) δ 12.58 (br. s, 1H), 8.30 (b s, 1H), 7.71 (s, 1H), 7.35 (d, J=9 Hz, 1H), 7.05 (d, J=9 Hz, 1H), 4.33 (t, J=6 Hz, 2H), 4.00 (t, J=6 Hz, 2H).

### EXAMPLE 99

### 6-(2,4-Dioxo-thiazolidin-5-ylidenemethyl)-benzo[1,4]oxazine-4-carboxylic acid tert-butyl ester

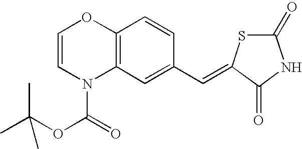

Following the general method as outlined in Example 1, starting from 6-Formyl-benzo[1,4]oxazine-4-carboxylic acid tert-butyl ester (intermediate 63) and 1,3-thiazolidine-2,4-dione, the title compound was obtained.

HPLC: 4.23 min. LC-MS: M/Z ESI: 1.82 min, 359.16 (M−1). ¹HNMR: (DMSO-d6) δ 12.50 (br. s, 1H), 7.63 (d, J=3 Hz, 2H), 7.31 (d, J=3 Hz, 1H), 6.95 (d, j=6 Hz, 1H), 6.30 (s, 2H).

### EXAMPLE 100

### [6-(2,4-Dioxo-thiazolidin-5-ylidenemethyl)-3-oxo-2,3-dihydro-benzo[1,4]-oxazin-4-yl]-acetic acid methyl ester

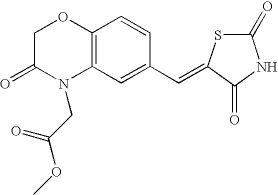

Following the general method as outlined in Example 1, starting from (6-Formyl-3-oxo-2,3-dihydro-benzo[1,4]oxazin-4-yl)-acetic acid methyl ester (intermediate 64) and 1,3-thiazolidine-2,4-dione, the title compound was obtained.

HPLC: 2.83 min. LC-MS: M/Z ESI: 1.20 min, 347.25 (M−1). ¹H NMR: (DMSO-d6) δ 12.58 (br. s, 1H), 7.76 (s, 1H), 7.36 (s, 1H), 7.20 (m, 2H), 4.82 (d, J=15 Hz, 4H), 3.71 (s, 3H).

### EXAMPLE 101

### N-Benzyl-2-[6-(2,4-dioxo-thiazolidin-5-ylidenemethyl)-3-oxo-2,3-dihydro-benzo[1,4]oxazin-4-yl]-acetamide

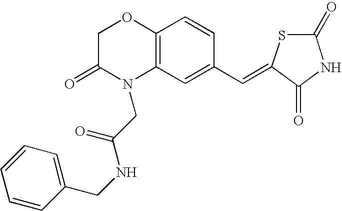

[6-(2,4-Dioxo-thiazolidin-5-ylidenemethyl)-3-oxo-2,3-dihydro-benzo[1,4]-oxazin-4-yl]-acetic acid methyl ester (195 mg, 0.56 mmol) (example 100) were saponified using 2 eq. of LiOH as described for example 74 affording [6-(2,4-Dioxo-thiazolidin-5-ylidenemethyl)-3-oxo-2,3-dihydro-benzo[1,4]oxazin-4-yl]-acetic acid. The so obtained acid (50 mg, 0.15 mmol) was dissolved in THF. HOBt (32 mg, 1.5 eq.), EDC (43 mg, 1.5 eq.) and benzylamine (25 mg, 1.5 eq.) were added while stirring. The reaction mixture was stirred for 15 h at rt. EtOAc was added and the organic phase was washed with 1N HCl, NaHCO₃, brine each of which three times. The crude residue after evaporating the solvents was purified on silica gel using DCM/EtOAc as eluents to give the title compound as colourless powder (35 mg, 54%).

HPLC: 3.06 min. LC-MS: M/Z ESI: 1.27 min, 424.21 (M+1).

### EXAMPLE 102

### 5-(4-Butyl-3-oxo-3,4-dihydro-2H-benzo[1,4]oxazin-6-ylmethylene)-thiazoli-dine-2,4-dione

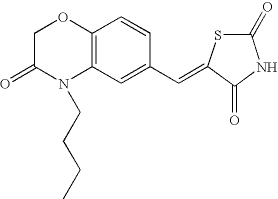

Following the general method as outlined in Example 1, starting from 4-Butyl-3-oxo-3,4-dihydro-2H-benzo[1,4]oxazine-6-carbaldehyde (intermediate 65) and 1,3-thiazolidine-2,4-dione, the title compound was obtained.

HPLC: 3.67 min. LC-MS: M/Z ESI: 1.49 min, 331.23 (M−1). ¹HNMR: (DMSO-d6) δ 12.58 (br. s, 1H), 7.85 (s, 1H), 7.43 (s, 1H), 7.24 (d, J=6 Hz, 1H), 7.15 (d, J=9 Hz, 1H), 4.73 (s, 2H), 3.91 (t, J=3 Hz, 2H), 1.57, (m, 2), 1.36 (m, 2H), 0.91 (t, J=9 Hz, 3H).

### EXAMPLE 103

### 5-(4-Benzyl-3-oxo-3,4-dihydro-2H-benzo[1,4]oxazin-6-ylmethylene)-thiazolidine-2,4-dione

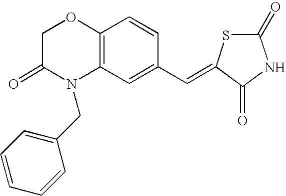

Following the general method as outlined in Example 1, starting from 4-Benzyl-3-oxo-3,4-dihydro-2H-benzo[1,4]oxazine-6-carbaldehyde (intermediate 66) and 1,3-thiazolidine-2,4-dione, the title compound was obtained.

HPLC: 3.67 min. LC-MS: M/Z ESI: 1.46 min, 365.17 (M−1). ¹H NMR: (DMSO-d6) δ 12.58 (br. s, 1H), 7.68 (s, 1H), 7.38-7.22 (m, 8H), 5.24 (s, 2H), 4.97 (s, 2H).

### EXAMPLE 104

### 5-(2-Chloro-benzofuran-5-ylmethylene)-thiazolidine-2,4-dione

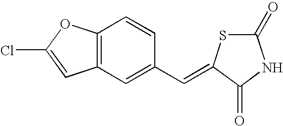

Following the general method as outlined in Example 1, starting from 2-Chloro-5-[1,3]dioxolan-2-yl-benzofurane (intermediate 67) and 1,3-thiazolidine-2,4-dione, the title compound was obtained.

HPLC: 3.84 min. LC-MS: M/Z ESI: 1.62 min, 278.12 (M−1). ¹H NMR: (DMSO-d6) δ 7.90-7.75 (M, 2H), 7.68 (d, j=9 Hz, 1H), 7.52 (d, J=9 Hz, 1H), 7.09 (s, 1H).

### EXAMPLE 105

### 5-(3-Amino-benzo[d]isoxazol-5-ylmethylene)-thiazolidine-2,4-dione

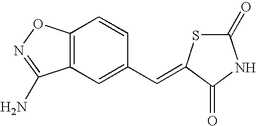

Following the general method as outlined in Example 1, starting from 3-Amino-benzo[d]isoxazole-5-carbaldehyde (intermediate 68) and 1,3-thiazolidine-2,4-dione, the title compound was obtained.

HPLC: 2.45 min. LC-MS: M/Z ESI: 0.97 min, 260.17 (M−1). ¹H NMR: (DMSO-d6) δ 12.60 (br. s, 1H), 8.01 (s, 1H), 7.85 (s, 1H), 7.60 (d, J=9 Hz, 1H), 6.67 (s, 1H).

### EXAMPLE 106

### 5-(3-Phenylethyl-benzofuran-5-ylmethylene)-thiazolidine-2,4-dione

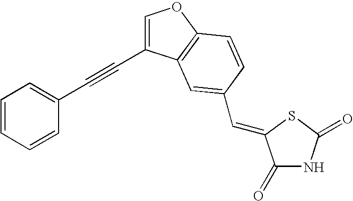

Following the general method as outlined in Example 1, starting from 3-Phenylethynyl-benzofuran-5-carbaldehyde (intermediate 59) and 1,3-thiazolidine-2,4-dione, the title compound was obtained.

HPLC: 4.82 min. LC-MS: M/Z ESI: 2.02 min, 344.18 (M−1). ¹H NMR: (DMSO-d6) δ 12.58 (br. s, 1H), 8.49 (s, 1H), 7.92 (s, 1H), 7.72 (d, J=9 Hz, 1H), 7.62 (m, 3H), 7.45 (m, 4H).

### EXAMPLE 107

### 5-Benzo[1,2,5]thiadiazol-5-ylmethylene-thiazolidine-2,4-ione

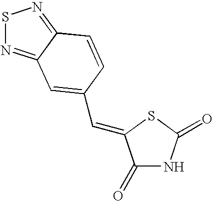

Following the general method as outlined in Example 1, starting from 2,1,3-Benzothiadiazole-5-carbaldehyde and 1,3-thiazolidine-2,4-dione, the title compound was obtained.

HPLC: 3.03 min. LC-MS: M/Z ESI: 1.14 min, 262.11 (M−1). ¹H NMR: (DMSO-d6) δ 12.58 (br. s, 1H), 8.11 (m, 2H), 7.90 (d, J=9 Hz, 1H), 7.47 (s, 1H).

### EXAMPLE 108

### 5-Benzo[1,2,5]oxadiazol-5-ylmethylene-thiazolidine-2,4-ione

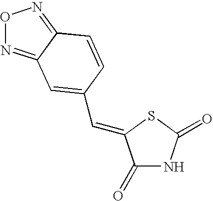

Following the general method as outlined in Example 1, starting from 2,1,3-Benzoxadiazole-5-carbaldehyde and 1,3-thiazolidine-2,4-dione, the title compound was obtained.

HPLC: 3.02 min. LC-MS: M/Z ESI: 1.17 min, 246.17 (M−1). ¹H NMR: (DMSO-d6) δ 12.58 (br. s, 1H), 8.07 (m, 2H), 7.82 (d, J=9 Hz, 1H), 7.40 (s, 1H).

### EXAMPLE 109

### 5-(2-Methyl-benzofuran-6-ylmethylene)-thiazolidine-2,4-dione

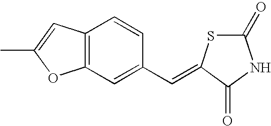

Following the general method as outlined in Example 1, starting from 2-Methyl-5-[1,3]dioxolan-2-yl-benzofuran (intermediate 72) and 1,3-thiazolidine-2,4-dione, the title compound was obtained after purification on reverse phase HPLC (solvents gradient H₂O/CH₃CN 0.1% TFA).

HPLC: 3.65 min, 90.75%. LC-MS: M/Z ESI: 1.65 min, 258.21 (M−1). ¹H NMR: (DMSO-d6) δ12.45 (s1, 1H), 7.88 (s, 1H), 7.77 (d, 1H, J=1.5 Hz), 7.64 (d, 1H, J=8.6 Hz), 7.47 (dd, 1H, J=8.6, 1.5 Hz), 6.69 (s, 1H), 2.37 (s, 3H).

### EXAMPLE 110

### 5-(2-Carboxymethyl-benzofuran-6-ylmethylene)-thiazolidine-2,4-dione

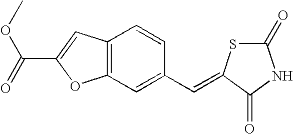

Following the general method as outlined in Example 1, starting from 5-[1,3]Dioxolan-2-yl-benzofuran-2-carboxylic acid methyl ester (intermediate 73) and 1,3-thiazolidine-2,4-dione, the title compound was obtained after purification on reverse phase HPLC (solvents gradient H₂O/CH₃CN 0.1% TFA).

HPLC: 3.32 min, 92.06%. LC-MS: M/Z ESI: 1.51 min, 302.19 (M−1). ¹HNMR: (DMSO-d6) δ12.52 (s1, 1H), 7.97 (d, 1H, J=1.5 Hz), 7.82 (m, 3H), 7.69 (dd, 1H, J=8.6, 1.5 Hz), 3.90 (s, 3H).

### EXAMPLE 111

### 5-(3-Bromo-2-fluoro-2,3-dihydro-benzofuran-6-ylmethylene)-thiazolidine-2,4-dione

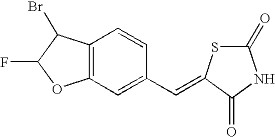

Following the general method as outlined in Example 1, starting from 3-Bromo-2-fluoro-benzofuran-5-carbaldehyde (intermediate 74) and 1,3-thiazolidine-2,4-dione, the title compound was obtained after purification on reverse phase HPLC (solvents gradient H₂O/CH₃CN 0.1% TFA).

HPLC: 3.66 min, 92.37%. LC-MS: M/Z ESI: 1.56 min, 343.09 (M−1). ¹H NMR: (DMSO-d6) δ12.82 (s1, 1H), 8.00 (d, 1H, J=1.8 Hz), 7.88 (dd, 1H, J=8.5, 1.8 Hz), 7.55 (d, 1H, J=8.5 Hz), 7.03 (d, 1H, ²J_{H-F}=590.5 Hz), 6.20 (d, 1H, ³J_{H-F}=15.3 Hz). ¹⁹F NMR: (DMSO-d6) δ−114.66.

### EXAMPLE 112

### 5-(2-Fluoro-benzofuran-6-ylmethylene)-thiazolidine-2,4-dione

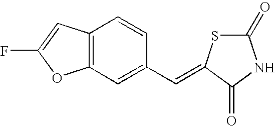

Following the general method as outlined in Example 1, starting from 2-Fluoro-5-D [1,3]dioxolan-2-yl-benzofuran (intermediate 75) and 1,3-thiazolidine-2,4-dione, the title compound was obtained after purification on reverse phase HPLC (solvents gradient H₂O/CH₃CN 0.1% TFA).

HPLC: 3.67 min, 99.47%. LC-MS: M/Z ESI: 1.51 min, 262.14 (M−1). ¹H NMR: (DMSO-d6) δ12.04 (s1, 1H), 7.89 (d, 1H, J=1.5 Hz), 7.83 (d, 1H, J=1.5 Hz), 7.73 (d, 1H, J=8.6 Hz), 7.55 (dd, 1H, J=8.6, 1.5 Hz), 6.47 (d, 1H, ³J_{H-F}=6.4 Hz). ¹⁹F NMR: (DMSO-d6) δ−111.28, −112.18.

### EXAMPLE 113

### Preparation of a Pharmaceutical Formulation

The following formulation examples illustrate representative pharmaceutical compositions according to the present invention being not restricted thereto.

Formulation 1—Tablets

A compound of formula (I) is admixed as a dry powder with a dry gelatin binder in an approximate 1:2 weight ration. A minor amount of magnesium stearate is added as a lubricant. The mixture is formed into 240-270 mg tablets (80-90 mg) of active azolidinone compound per tablet) in a tablet press.

Formulation 2—Capsules

A compound of formula (I) is admixed as a dry powder with a starch diluent in an approximate 1:1 weight ratio. The mixture is filled into 250 mg capsules (125 mg of active azolidinone compound per capsule).

Formulation 3—Liquid

A compound of formula (I) (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

Formulation 4—Tablets

A compound of formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active azolidinone compound) in a tablet press.

Formulation 5—Injection

A compound of formula (I) is dissolved in a buffered sterile saline injectable aqueous medium to a concentration of approximately 5 mg/ml.

### EXAMPLE 14

### Biological Assays

A. a) High Throughput PI3K Lipid Kinase Assay (Binding Assay):

The assay combines the scintillation proximity assay technology (SPA, Amersham) with the capacity of neomycin (a polycationic antibiotic) to bind phospholipids with high affinity and specificity. The Scintillation Proximity Assay is based on the properties of weakly emitting isotopes (such as ³H, ¹²⁵I, ³³P). Coating SPA beads with neomycin allows the detection of phosphorylated lipid substrates after incubation with recombinant PI3K and radioactive ATP in the same well, by capturing the radioactive phospholipids to the SPA beads through their specific binding to neomycin.

To a 384 wells MTP containing 5 μl of the test compound of formula (I) (solubilized in 6% DMSO; to yield a concentration of 100, 30, 10, 3, 1, 0.3, 0.1, 0.03, 0.01, 0.001 μM of the test compound), the following assay components are added. 1) 5 μl (58 ng) of Human recombinant GST-PI3Kγ (in Hepes 40 mM, pH 7.4, DTT 1 mM and ethylenglycol 5%) 2) 10 μl of lipid micelles and 3) 10 μl of Kinase buffer ([³³P]ATP 45 μM/60 nCi, MgCl₂ 30 mM, DTT 1 mM, β-Glycerophosphate 1 mM, Na₃VO₄ 100 μM, Na Cholate 0.3%, in Hepes 40 mM, pH 7.4). After incubation at room temperature for 180 minutes, with gentle agitation, the reaction is stopped by addition of 60 μl of a solution containing 100 μg of neomycin-coated PVT SPA beads in PBS containing ATP 10 mM and EDTA 5 mM. The assay is further incubated at room temperature for 60 minutes with gentle agitation to allow binding of phospholipids to neomycin-SPA beads. After precipitation of the neomycin-coated PVT SPA beads for 5 minutes at 1500×g, radioactive PtdIns(3)P is quantified by scintillation counting in a Wallac MicroBeta™ plate counter.

The values indicated in respect of PI3Kγ refer to the IC₅₀ (μM), i.e. the amount necessary to achieve 50% inhibition of said target. Said values show a considerable potency of the azolidinone-vinyl fused-benzene compounds with regard to PI3Kγ.

The tested compounds according to formula (I) display an inhibition (IC₅₀) with regard to PI3Kγ of less than 2 μM, more preferred equal or less than 1 μM.

Examples of inhibitory activities for test compounds 41, 61, 66, 73, 103, 107, and 110 as set out in Table 1.

TABLE 1
IC₅₀ values of azolidinone-vinyl
fused-benzene derivatives against PI3Kγ.

| Example No | PI3Kγ, IC₅₀ (μM) |
|---|---|
| 41 | <1 |
| 61 | <1 |
| 66 | <1 |
| 73 | <1 |
| 103 | <1 |
| 107 | <1 |
| 110 | <1 |

b) Cell Based ELISA to Monitor PI3K Inhibition:

Measurement of Akt/PKB phosphorylation in macrophages after stimulation with C5a: Raw 264: Raw 264-7 macrophages (cultured in DMEM-F12 medium containing 10% Fetal Calf serum and antibiotics) are plated at 20'000 cells/well in a 96 MTP 24 h before cell stimulation. Previous to the stimulation with 50 nM of Complement 5a (C5a; which is a well known chemokine which stimulates the used cells) during 5 minutes, Cells are serum starved for 2 h, and pretreated with inhibitors for 20 minutes. After stimulation cells are fixed in 4% formaldehyde for 20 minutes and washed 3 times in PBS containing 1% Triton X-100 (PBS/Triton). Endogenous peroxidase is blocked by a 20 minutes incubation in 0.6% H₂O₂ and 0.1% Sodium Azide in PBS/Triton and washed 3 times in PBS/Triton. Cells are then blocked by 60 minutes incubation with 10% fetal calf serum in PBS/Triton. Next, phosphorylated Akt/PKB is detected by an overnight incubation at 4° C. with first antibody (anti phospho Serine 473 Akt IHC, Cell Signaling) diluted 800-fold in PBS/Triton, containing 5% bovine serum albumin (BSA). After 3 washes in PBS/Triton, cells are incubated for 60 minutes with a peroxidase conjugated goat-anti-rabbit antibody (1/400 dilution in PBS/Triton, containing 5% BSA), washed 3 times in PBS/Triton, and 2 times in PBS and further incubated in 100 μl of substrate reagent solution (R&D) for 20 minutes. The reaction is stopped by addition of 50 μl of 1 M H₂SO₄ and absorbance is read at 450 nm.

The values indicated reflect the percentage of inhibition of AKT phoshorylation as compared to basal level. Said values show a clear effect of the azolidinone-vinyl fused-benzene compounds on the activation of AKT phosphorylation in macrophages.

Compounds of examples 1, 19, 66 and 107, when used at 10 μM completely (about 100%) inhibit C5a-mediated AKT phosophorylation. Examples 17, 19 or 73, when used at 1 μM, inhibit 95% of the C5a-mediated AKT-phosphorylation.

B. In Vitro Experiments:

In the experiments the following examples are based on, standard methods of in vitro fertilization have been used. With regard to the details of these methods, reference is made to the “WHO manual” (WHO laboratory manual for the examination of human semen and sperm-cervical mucus interactions, 4^{th} edition, Cambridge University Press 1999). In particular, the direct swim-up method can be taken from pp. 104 to 106 of this manual.

a) Effect of the PI3K Inhibitor on the Rapid Motility of Spermatozoa:

Spermatozoa are prepared according to the standard procedures of IVF. Briefly, spermatozoa are prepared from 3 oligoasthenospermic subjects undergoing semen analysis for couple infertility after informed consent. 10 μM of the tested compound of formula (I) are added directly to the seminal liquid and incubated for 2 hours for two hours at 37° C. and 5% CO₂. The motility of the spermatozoa is then blindly evaluated under the microscope according to WHO manual procedures.

In a group of 7 samples taken from seven individuals, the tested phosphatidylinositol-3-kinase inhibitor is added in a higher concentration (100 μM). After the incubation with the compound for two hours, swim-up selection of the spermatozoa is performed according to procedures described in the WHO-manual. The incubation of the sperm cells with a ten times higher concentration of the compound of formula (I) (100 μM) in combination with the swim-up selection results in a significant increase of progressive motility in all of the seven samples.

Results may be obtained in a similar experiment on samples from higher numbers of patients. The sperm cells are submitted to the swim-up selection method. Treatment with 10 μM of the tested phosphatidylinositol-3-kinase inhibitor results in an increase in the progressive motility as compared to the control (patients without LY294002). Treatment of samples from patients with 100 μM of the inhibitor results in an increase of the motility as compared to the control.

The effect of 100 μM of the compound of formula (I) on the viability of the spermatozoa is also assessed. The incubation with the tested PI3K inhibitor is carried out to observe the alteration of the vitality of the cells for two hours and after 48 hours.

Further experiment may be carried out in the same manner as outlined above on samples from 12 individual patients.

b) Effect of Example 1 Compound on Further Sperm Cell Parameters:

The increase in forward motility, demonstrated in the above mentioned part A, is associated with an increase in sperm parameters related to fertilization activity of the spermatozoa in vitro, such as percentage curvilinear velocity (VCL), average path velocity (VAP), straight-line velocity (VSL) and hyper-activated sperm fraction (HA). These parameters are determined by computer aided sperm analysis (CASA) in sperm samples from different oligo-asthenospermic subjects. Each of these parameters are increased in a statistically significant manner by incubation with 10 μM of compound of example 1 as compared to the control sample, indicating a significant overall improvement of sperm cell fertilization activity.

c) Effect of the Tested Compound of Formula (I) on Forward Motility of H₂O₂ or LiCl Treated Spermatozoa:

It is well known that reactive oxygen species (ROS), which may be generated during sperm preparation for IVF, exert detrimental effects on sperm fertilization potential. In particular, among ROS, H₂O₂ strongly reduces motility when added to sperm samples at micromolar concentrations. Therefore, the effect of the tested compound of formula (I) on H₂O₂ treated sperm cells is evaluated. The compound is added to swim-up selected spermatozoa samples from oligo-asthenospermic patients in amounts of 10 μM either alone or in combination with 200 μM of H₂O₂.

LiCl is known as having inhibition properties on sperm cell motility. An incubation of swim-up selected spermatozoa with 10 μM of tested compound of formula (I) for two hours either with or without different concentrations of LiCl results in reversing the effect of LiCl induced inhibition of sperm motility.

In this example, the activity of compounds of formula (I) to rescue spermatozoa from deleterious agents, which may be generated in assisted fertilization techniques has been demonstrated. Therefore, the invention provides for a major improvement of ART, leading to a higher fertilization rate and eliminating some of the most serious drawbacks of these techniques.

1. A process for improving the fertilization activity of spermatozoa, in particular for increasing spermatozoa motility, comprising the step of treating spermatozoa with a compound of formula (I) 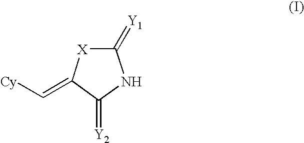
as well as its geometrical isomers, its optically active forms as enantiomers, diastereomers and its racemate forms, as well as pharmaceutically acceptable salts and pharmaceutically active derivatives thereof, wherein
X is S, O or NH;
Y¹ and Y² are independently S, O or —NH;
Cy is a 5 to 8 membered carbocyclic or heterocyclic group may be fused with an aryl, heteroaryl, cycloalkyl or heterocycloalkyl group.

2. The process according to claim 1, whereby the compound has formula (I′) 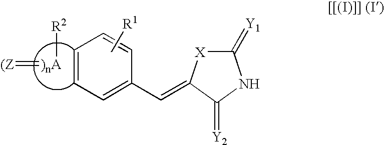
A is a 5-8 membered heterocyclic or carbocyclic group, wherein said carbocyclic group may be fused with aryl, heteroaryl, cycloalkyl or heterocycloalkyl;
X is S, O or NH;
Y¹ and Y² are independently S, O or —NH;
Z is S or O;
R¹ is H, CN, carboxy, acyl, C₁-C₆-alkoxy, halogen, hydroxy, acyloxy, C₁-C₆-alkyl carboxy, C₁-C₆-alkyl acyloxy, C₁-C₆-alkyl alkoxy, alkoxycarbonyl, C₁-C₆-alkyl alkoxycarbonyl, aminocarbonyl, C₁-C₆-alkyl aminocarbonyl, acylamino, C₁-C₆-alkyl acylamino, ureido, C₁-C₆-alkyl ureido, amino, C₁-C₆-alkyl amino, ammonium, sulfonyloxy, C₁-C₆-alkyl sulfonyloxy, sulfonyl, C₁-C₆-alkyl sulfonyl, sulfinyl, C₁-C₆-alkyl sulfinyl, sulfanyl, C₁-C₆-alkyl sulfanyl, sulfonylamino, C₁-C₆-alkyl sulfonylamino or carbamate;
R² is selected from the group comprising or consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, C₃-C₈-cycloalkyl or heterocycloalkyl, C₁-C₆-alkyl aryl, C₂-C₆-alkenyl-aryl, C₂-C₆-alkynyl aryl, carboxy, cyano, hydroxy, C₁-C₆-alkoxy, nitro, acylamino, ureido, C₁-C₆-alkyl carbamate, sulfonylamino, sulfanyl, or sulfonyl;
n is 0, 1 or 2;

3. The process according to claim 1, wherein the compound is selected from any of formulae (Ia), (Ib), (Ic) or (Id) 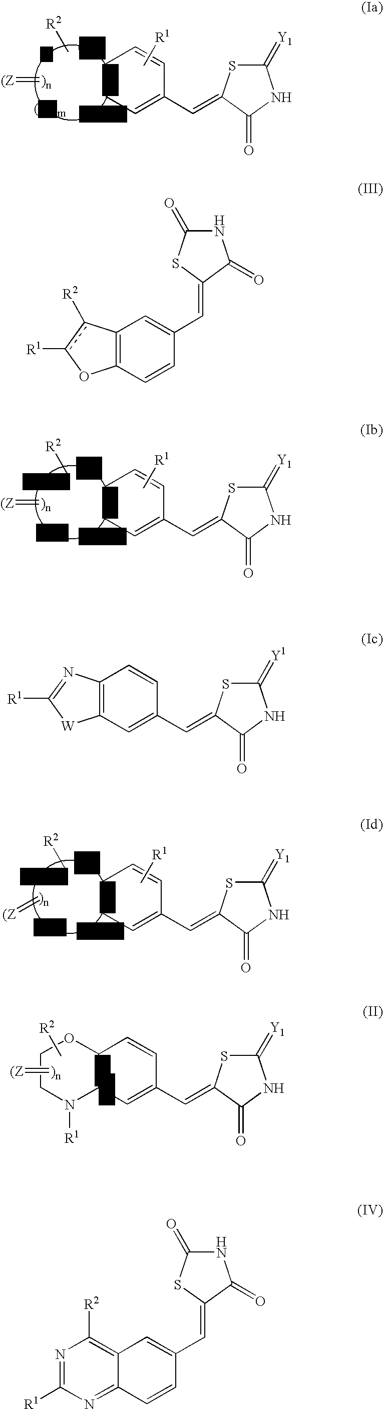 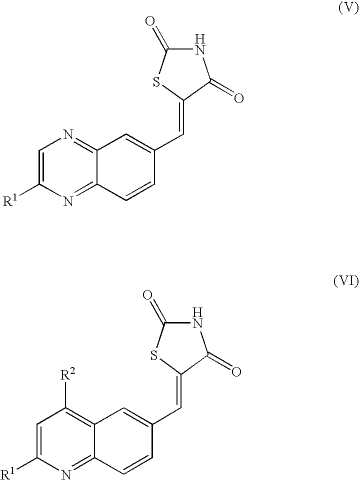
wherein R¹, R², Y¹, Z and n are as above-defined, G is a C₁-C₅ alkylene or a C₁-C₅ alkenylene group, W and V are each independently from each other selected from O, S, —NR³ wherein R³ is H or a C₁-C₆ alkyl group, m, n and o are each independently from each other 0 or 1, p and q are independently from each other an integer from 1 to 4.

4. The process according to claim 1, wherein the compound has formula (II) 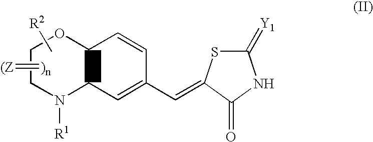
wherein Z, Y¹, R¹, R² are as above defined; n is 0 or 1.

5. The process according to claim 1, wherein the compound has formula (III) 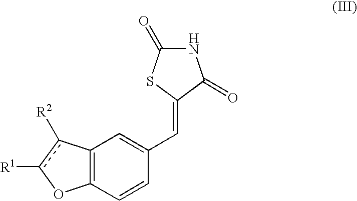
wherein R¹ and R² are as above defined.

6. The process according to claim 1, wherein the compound has any of formulae (IV), (V) or (VI); 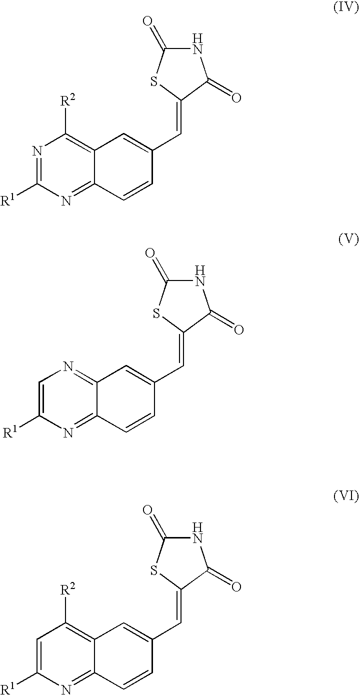
wherein R¹ and R² are as above defined.

7. The process according to claim 1, wherein treating the spermatozoa with the compound of formula (I) is performed on seminal liquid comprising the spermatozoa.

8. The process according to claim 1, further comprising separating the spermatozoa by spermatozoa separation methods used in assisted reproduction techniques.

9. The process according to claim 8, wherein separating the spermatozoa is performed by a method selected from the wash and spin method, the sedimentation method, the direct swim-up method, the pellet and swim-up method, the buoyant density gradient method.

10. The process according to claim 9, wherein separating the spermatozoa is performed by the direct swim-up method.

11. The process according to claim 1, wherein the process is performed on mammal spermatozoa, in particular on human spermatozoa.

12. The process according to claim 1, wherein the compound of formula (I) is selected from the group consisting of:
5-(1,3-benzodioxol-5-ylmethylene)-1,3-thiazolidine-2,4-dione
5-(1,3-benzodioxol-5-ylmethylene)-2-thioxo-1,3-thiazolidin-4-one
5-(2,3-dihydro-1,4-benzodioxin-6-ylmethylene)-1,3-thiazolidine-2,4-dione
5-(2,3-dihydro-1-benzofuran-5-ylmethylene)-1,3-thiazolidine-2,4-dione
5-[(7-methoxy-1,3-benzodioxol-5-yl)methylene]-1,3-thiazolidine-2,4-dione
5-[(9,10-dioxo-9,10-dihydroanthracen-2-yl)methylene]-1,3-thiazolidine-2,4-dione
(5-[(2,2-difluoro-1,3-benzodioxol-5-yl)methylene]-1,3-thiazolidine-2,4-dione
(5Z)-5-(1,3-dihydro-2-benzofuran-5-ylmethylene)-1,3-thiazolidine-2,4-dione
5-(1-benzofuran-5-ylmethylene)-1,3-thiazolidine-2,4-dione
5-[(4-methyl-3-oxo-3,4-dihydro-2H-1,4-benzoxazin-6-yl)methylene]-1,3-thiazolidine-2,4-dione
5-(1,3-benzodioxol-5-ylmethylene)-2-imino-1,3-thiazolidin-4-one
5-Quinolin-6-ylmethylene-thiazolidine-2,4-dione
5-Quinolin-6-ylmethylene-2-thioxo-thiazolidin-4-one
2-Imino-5-quinolin-6-ylmethylene-thiazolidin-4-one
5-(3-Methyl-benzo[d]isoxazol-5-ylmethylene)-thiazolidine-2,4-dione
5-(4-Phenyl-quinazolin-6-ylmethylene)-thiazolidine-2,4-dione
5-(4-Dimethylamino-quinazolin-6-ylmethylene)-thiazolidine-2,4-dione
5-[(4-aminoquinazolin-6-yl)methylene]-1,3-thiazolidine-2,4-dione
5-[(4-piperidin-1-ylquinazolin-6-yl)methylene]-1,3-thiazolidine-2,4-dione
5-[(4-morpholin-4-ylquinazolin-6-yl)methylene]-1,3-thiazolidine-2,4-dione
5-{[4-(benzylamino)quinazolin-6-yl]methylene}-1,3-thiazolidine-2,4-dione
5-{[4-(diethylamino)quinazolin-6-yl]methylene}-1,3-thiazolidine-2,4-dione
5-({4-[(pyridin-2-ylmethyl)amino]quinazolin-6-yl}methylene)-1,3-thiazolidine-2,4-dione
5-({4-[(pyridin-3-ylmethyl)amino]quinazolin-6-yl}methylene)-1,3-thiazolidine-2,4-dione
ethyl 1-{6-[(2,4-dioxo-1,3-thiazolidin-5-ylidene)methyl]quinazolin-4-yl}piperidine-3-carboxylate
ethyl 1-{6-[(2,4-dioxo-1,3-thiazolidin-5-ylidene)methyl]quinazolin-4-yl}piperidine-4-carboxylate
tert-butyl 1-{6-[(2,4-dioxo-1,3-thiazolidin-5-ylidene)methyl]quinazolin-4-yl}-L-prolinate
5-{[4-(4-methylpiperazin-1-yl)quinazolin-6-yl]methylene}-1,3-thiazolidine-2,4-dione
5-{[4-(4-pyrimidin-2-ylpiperazin-1-yl)quinazolin-6-yl]methylene}-1,3-thiazolidine-2,4-dione
5-({4-[4-(4-fluorophenyl)piperidin-1-yl]quinazolin-6-yl}methylene)-1,3-thiazolidine-2,4-dione
5-{[4-(4-benzylpiperidin-1-yl)quinazolin-6-yl]methylene}-1,3-thiazolidine-2,4-dione
5-({4-[4-(2-phenylethyl)piperidin-1-yl]quinazolin-6-yl}methylene)-1,3-thiazolidine-2,4-dione
5-{[4-(4-methylpiperidin-1-yl)quinazolin-6-yl]methylene}-1,3-thiazolidine-2,4-dione
5-{[4-(4-hydroxypiperidin-1-yl)quinazolin-6-yl]methylene}-1,3-thiazolidine-2,4-dione
1-[6-(2,4-Dioxo-thiazolidin-5-ylidenemethyl)-quinazolin-4-yl]-piperidine-4-carboxylic acid
1-[6-(2,4-Dioxo-thiazolidin-5-ylidenemethyl)-quinazolin-4-yl]-piperidine-3-carboxylic acid
1-[6-(2,4-Dioxo-thiazolidin-5-ylidenemethyl)-quinazolin-4-yl]-pyrrolidine-2-carboxylic acid
5-(4-Methylamino-quinazolin-6-ylmethylene)-thiazolidine-2,4-dione
5-(4-Methoxy-quinazolin-6-ylmethylene)-thiazolidine-2,4-dione
2-Imino-5-(4-methylamino-quinazolin-6-ylmethylene)-thiazolidin-4-one
2-Imino-5-(4-piperidine-quinazolin-6-ylmethylene)-thiazolidin-4-one
2-Imino-5-(4-dimethylamino-quinazolin-6-ylmethylene)-thiazolidin-4-one
5-(2-Methyl-2H-benzotriazol-5-ylmethylene)-thiazolidine-2,4-dione
5-(3-Methyl-3H-benzotriazol-5-ylmethylene)-thiazolidine-2,4-dione
5-(3-Ethyl-3H-benzoimidazol-5-ylmethylene)-thiazolidine-2,4-dione
5-{[1-(4-phenylbutyl)-1H-benzimidazol-6-yl]methylene}-1,3-thiazolidine-2,4-dione
5-[(1-prop-2-yn-1-yl-1H-benzimidazol-6-yl)methylene]-1,3-thiazolidine-2,4-dione
5-[(1-{2-[4-(trifluoromethyl)phenyl]ethyl}-1H-benzimidazol-6-yl)methylene]-1,3-thiazolidine-2,4-dione
5-({1-[2-(4-hydroxyphenyl)ethyl]-1H-benzimidazol-6-yl}methylene)-1,3-thiazolidine-2,4-dione
methyl 4-{6-[(2,4-dioxo-1,3-thiazolidin-5-ylidene)methyl]-1H-benzimidazol-1-yl}cyclohexanecarboxylate
5-({1-[2-(5-methoxy-1H-indol-3-yl)ethyl]-1H-benzimidazol-6-yl}methylene)-1,3-thiazolidine-2,4-dione
5-({1-[(1-methyl-1H-pyrazol-4-yl)methyl]-1H-benzimidazol-6-yl}methylene)-1,3-thiazolidine-2,4-dione
5-({1-[2-(3,4-dimethoxyphenyl)ethyl]-1H-benzimidazol-6-yl}methylene)-1,3-thiazolidine-2,4-dione
5-({1-[2-(4-phenoxyphenyl)ethyl]-1H-benzimidazol-6-yl}methylene)-1,3-thiazolidine-2,4-dione
5-({1-[4-(trifluoromethyl)benzyl]-1H-benzimidazol-6-yl}methylene)-1,3-thiazolidine-2,4-dione
4-{6-[(2,4-dioxo-1,3-thiazolidin-5-ylidene)methyl]-1H-benzimidazol-1-yl}cyclohexanecarboxylic acid
5-[(1-isobutyl-1H-benzimidazol-6-yl)methylene]-1,3-thiazolidine-2,4-dione
5-({1-[2-(1,3-benzodioxol-4-yl)ethyl]-1H-benzimidazol-6-yl}methylene)-1,3-thiazolidine-2,4-dione
5-({1-[2-(2-phenoxyphenyl)ethyl]-1H-benzimidazol-6-yl}methylene)-1,3-thiazolidine-2,4-dione
5-{[1-(3,3-diphenylpropyl)-1H-benzimidazol-6-yl]methylene}-1,3-thiazolidine-2,4-dione
5-{[1-(2-methoxybenzyl)-1H-benzimidazol-6-yl]methylene}-1,3-thiazolidine-2,4-dione
5-{[1-(3-furylmethyl)-1H-benzimidazol-6-yl]methylene}-1,3-thiazolidine-2,4-dione
5-[(1-propyl-1H-benzimidazol-6-yl)methylene]-1,3-thiazolidine-2,4-dione
5-Quinoxalin-6-ylmethylene-thiazolidine-2,4-dione
5-Quinoxalin-6-ylmethylene-2-thioxo-thiazolidin-4-one
2-Imino-5-quinoxalin-6-ylmethylene-thiazolidin-4-one
5-Benzothiazol-6-ylmethylene-thiazolidine-2,4-dione
5-(3-Methyl-benzofuran-5-ylmethylene)-thiazolidine-2,4-dione
5-(2-Bromo-3-methyl-benzofuran-5-ylmethylene)-thiazolidine-2,4-dione
5-(3-bromo-benzofuran-5-ylmethylene)-thiazolidine-2,4-dione
3-[5-(2,4-Dioxo-thiazolidin-5-ylidenemethyl)-benzofuran-3-yl]-acrylic acid ethyl ester
3-[5-(2,4-Dioxo-thiazolidin-5-ylidenemethyl)-benzofuran-3-yl]-acrylic acid
5-[3-(3-Oxo-3-piperidin-1-yl-propenyl)-benzofuran-5-ylmethylene]-thiazoli-dine-2,4-dione
Methyl 1-((3-{5-[(2,4-dioxo-1,3-thiazolidin-5-ylidene)methyl]-1-benzofuran-3-yl}prop-2-enoyl)prolinate
Methyl 1-((3-{5-[(2,4-dioxo-1,3-thiazolidin-5-ylidene)methyl]-1-benzofuran-3-yl}prop-2-enoyl)-D-prolinate
(5-({3-[(3-oxo-3-pyrrolidin-1-ylprop-1-en-1-yl]-1-benzofuran-5-yl}methylene)-1,3-thiazolidine-2,4-dione
5-({3-[3-morpholin-4-yl-3-oxoprop-1-en-1-yl]-1-benzofuran-5-yl}methylene)-1,3-thiazolidine-2,4-dione
Methyl 1-(3-{5-[(2,4-dioxo-1,3-thiazolidin-5-ylidene)methyl]-1-benzofuran-3-yl}prop-2-enoyl)-L-prolinate
N-cyclohexyl-3-{5-[(2,4-dioxo-1,3-thiazolidin-5-ylidene)methyl]-1-benzofuran-3-yl}-N-methylacrylamide
3-{5-[(2,4-dioxo-1,3-thiazolidin-5-ylidene)methyl]-1-benzofuran-3-yl}-N-ethyl-N-(2-hydroxyethyl)acrylamide
N-cyclobutyl-3-{5-[(2,4-dioxo-1,3-thiazolidin-5-ylidene)methyl]-1-benzofuran-3-yl}acrylamide
5-({3-[3-azetidin-1-yl-3-oxoprop-1-en-1-yl]-1-benzofuran-5-yl}methylene)-1,3-thiazolidine-2,4-dione
5-({3-[3-(1,3-dihydro-2H-isoindol-2-yl)-3-oxoprop-1-en-1-yl]-1-benzofuran-5-yl}methylene)-1,3-thiazolidine-2,4-dione
5-({3-[3-azepan-1-yl-3-oxoprop-1-en-1-yl]-1-benzofuran-5-yl}methylene)-1,3-thiazolidine-2,4-dione
3-{5-[(2,4-dioxo-1,3-thiazolidin-5-ylidene)methyl]-1-benzofuran-3-yl}-N-piperidin-1-ylacrylamide
3-{5-[(2,4-dioxo-1,3-thiazolidin-5-ylidene)methyl]-1-benzofuran-3-yl}-N-(pyridin-3-ylmethyl)acrylamide
N-cyclohexyl-3-{5-[(2,4-dioxo-1,3-thiazolidin-5-ylidene)methyl]-1-benzofuran-3-yl}acrylamide
5-({3-[3-(4-methylpiperazin-1-yl)-3-oxoprop-1-en-1-yl]-1-benzofuran-5-yl}methylene)-1,3-thiazolidine-2,4-dione
N-cycloheptyl-3-{5-[(2,4-dioxo-1,3-thiazolidin-5-ylidene)methyl]-1-benzofuran-3-yl}acrylamide
5-({3-[3-(2,5-dihydro-1H-pyrrol-1-yl)-3-oxoprop-1-en-1-yl]-1-benzofuran-5-yl}methylene)-1,3-thiazolidine-2,4-dione
N-cyclopentyl-3-{5-[(2,4-dioxo-1,3-thiazolidin-5-ylidene)methyl]-1-benzofuran-3-yl}acrylamide
3-[5-(2,4-Dioxo-thiazolidin-5-ylidenemethyl)-benzofuran-3-yl]-propionic acid ethyl ester
3-[5-(2,4-Dioxo-thiazolidin-5-ylidenemethyl)-benzofuran-3-yl]-propionic acid
5-[3-(3-Oxo-3-piperidin-1-yl-propyl)-benzofuran-5-ylmethylene]-thiazol-idine-2,4-dione
6-(2,4-Dioxo-thiazolidin-5-ylidenemethyl)-2,3-dihydro-benzo[1,4]oxazine-4-carboxylic acid tert-butyl ester
5-(3,4-Dihydro-2H-benzo[1,4]oxazin-6-ylmethylene)-thiazolidine-2,4-dione
5-(4-Benzoyl-3,4-dihydro-2H-benzo[1,4]oxazin-6-ylmethylene)-thiazolidine-2,4-dione
5-(4-Acetyl-3,4-dihydro-2H-benzo[1,4]oxazin-6-ylmethylene)-thiazolidine-2,4-dione
6-(2,4-Dioxo-thiazolidin-5-ylidenemethyl)-benzo[1,4]oxazine-4-carboxylic acid tert-butyl ester
[6-(2,4-Dioxo-thiazolidin-5-ylidenemethyl)-3-oxo-2,3-dihydro-benzo[1,4]-oxazin-4-yl]-acetic acid methyl ester
N-Benzyl-2-[6-(2,4-dioxo-thiazolidin-5-ylidenemethyl)-3-oxo-2,3-dihydro-benzo[1,4]oxazin-4-yl]-acetamide
5-(4-Butyl-3-oxo-3,4-dihydro-2H-benzo[1,4]oxazin-6-ylmethylene)-thiazolidine-2,4-dione
5-(4-Benzyl-3-oxo-3,4-dihydro-2H-benzo[1,4]oxazin-6-ylmethylene)-thiazolidine-2,4-dione
5-(2-Chloro-benzofuran-5-ylmethylene)-thiazolidine-2,4-dione
5-(3-Amino-benzo[d]isoxazol-5-ylmethylene)-thiazolidine-2,4-dione
5-(3-Phenylethynyl-benzofuran-5-ylmethylene)-thiazolidine-2,4-dione
5-Benzo[1,2,5]thiadiazol-5-ylmethylene-thiazolidine-2,4-dione
5-Benzo[1,2,5]oxadiazol-5-ylmethylene-thiazolidine-2,4-dione
5-(2-Methyl-benzofuran-6-ylmethylene)-thiazolidine-2,4-dione
5-(2-Carboxymethyl-benzofuran-6-ylmethylene)-thiazolidine-2,4-dione
5-(3-Bromo-2-fluoro-2,3-dihydro-benzofuran-6-ylmethylene)-thiazolidine-2,4-dione
5-(2-Fluoro-benzofuran-6-ylmethylene)-thiazolidine-2,4-dione

13. The process according to claim 1, wherein said spermatozoa are treated with an amount of a compound of formula (I) in the range of about 0.01 to 1000 μM, about 5 to 500 μM, or about 10 to 100 μM.

14. The process according to claim 1, wherein treating the spermatozoa with a compound of formula (I) comprises incubating the spermatozoa for a period of time in the range of about 30 minutes to 10 hours or about 1 to 8 hours or about 2 to 6 hours at a temperature of around 37° C.

15. Spermatozoa obtained by the process according to claim 1.

16. A method for improving the fertilization rate in assisted reproduction techniques comprising utilizing a compound of formula (I) in assisted reproduction techniques.

17. The method according to claim 16, wherein the assisted reproduction techniques are selected from in vitro fertilization (IVF), gamete intrafallopian transfer (GIFT), or intra-uterine insemination (IUI).

18. A method for the preparation of a pharmaceutical composition for the treatment of infertility comprising adding a compound of formula (I) to a pharmaceutical composition.

19. A method for the preparation of a pharmaceutical composition for improving spermatozoa fertilization activity, comprising adding a compound of formula (I) to a pharmaceutical composition.

20. A method of ART therapy, comprising treating spermatozoa with a compound of formula (I).

21. A method according to claim 20, wherein said ART therapy is selected from the group consisting of in vitro fertilization (IVF), gamete intrafallopian transfer (GIFT), or intra-uterine insemination (IUI).

22. A medium for storage and/or transportation of spermatozoa comprising a compound of formula (I).

23. The medium according to claim 22 for the storage and/or transportation of mammal spermatozoa.

24. The medium according to claim 22, comprising an amount of a compound of formula (I) in the range of about 0.01 to 1000 μM, about 5 to 500 μM, or about 10 to 100 μM.

25. The method according to claim 18 wherein said treatment of infertility is treatment of male infertility.

26. The method according to claim 19 wherein said improving spermatozoa fertilization activity is increasing spermatozoa motility.

27. The medium according to claim 23 wherein the mammal spermatozoa is human spermatozoa.
